(19) United States
(12) Patent Application Publication
(10) Pub. No.: US 2023/0203440 A1
(43) Pub. Date: Jun. 29, 2023

(54) AXIAL STEM CELLS, METHODS OF PRODUCING AND USES THEREOF

(71) Applicant: HELMHOLTZ ZENTRUM MUNCHEN - DEUTSCHES FORSCHUNGSZENTRUM FUR GESUNDHEIT UND UMWELT (GMBH), Neuherberg (DE)

(72) Inventors: Micha DRUKKER, Den Haag (NL); Dmitry SHAPOSHNIKOV, Munich (DE); Aicha Haji ALI, Planegg (DE); Dolunay KELLE-OZDEMIR, Munich (DE); Ejona RUSHA, Munich (DE)

(73) Assignee: HELMHOLTZ ZENTRUM MUNCHEN - DEUTSCHES FORSCHUNGSZENTRUM FUR GESUNDHEIT UND UMWELT (GMBH), Neuherberg (DE)

(21) Appl. No.: 17/927,365

(22) PCT Filed: May 25, 2021
(86) PCT No.: PCT/EP2021/063843

§ 371 Date: Nov. 23, 2022

(30) Foreign Application Priority Data
May 25, 2020 (EP) 20176375.2
May 27, 2020 (LU) LU101823

(51) Int. Cl. C12N 5/0797; A61K 35/30

(52) U.S. Cl. CPC C12N 5/0623; A61K 35/30; C12N 2506/45; C12N 2501/115; C12N 2501/15; C12N 2501/727; C12N 2501/415

(57) ABSTRACT

The present invention relates to methods of producing axial stem cells (AxSCs) as well to axial stem cells (AxSCs) produced by such methods and uses thereof. The present invention further relates to axial stem cells (AxSCs), wherein said axial stem cells are not pluripotent cells, but are, for example, region-specific multipotent stem cells capable of indefinitely renewing themselves.

This application contains a Sequence Listing in computer readable form, which is incorporated herein by reference.

### TECHNICAL FIELD

The present invention relates to methods for producing axial stem cells (AxSCs) as well to axial stem cells (AxSCs), e.g., produced by such methods, and uses thereof. The present invention further relates to axial stem cells (AxSCs) having one or more of the following characteristics: not pluripotent cells; region-specific multipotent stem cells; obtainable from pluripotent stem cells; not capable of differentiating into cell types of all tissues of the embryo; only capable of differentiating into cell types emerging from the region of the central body axis during embryonic development (e.g., sclerotome, dermomyotome and peripheral neurons); not capable to form teratomas; capable of mimicking the characteristics of the precursors that give rise to the axial region (e.g., motor neurons, peripheral neurons, peripheral nervous system neurons sensory neurons, bone, cartilage, tendon, ligament and/or skeletal muscle cells); not transient cells; capable of differentiating into a motor neuron, peripheral neuron, muscle, cartilage or bone progenitor; indefinitely renewing stem cells; capable to grow as clones.

### BACKGROUND OF THE INVENTION

A remarkable hallmark of the development of mammal organisms is the ability of embryonic cells to self-renew for a defined period of time, which maintains distinct pools of stem cells, while producing daughter progeny undergoing differentiation into various tissue types. Identification of mechanisms that promote proliferation and inhibit differentiation of pluripotent, trophoblast and extraembryonic endoderm cells in the mouse blastocyst have led to informed design of cell culture environments that promote the derivation of indefinitely renewing stem cell lines, named ESCs/PSCs, TSCs and XENs, respectively (Martin, 1981; Evans and Kaufman, 1981; Tanaka et al., 1998; Niakan et al., 2013). Furthermore, pluripotent lines that correspond to pre- and post-implantation blastocyst, named naive/ground and primed states respectively, as well as recently proposed formative pluripotency state (Smith, 2017), have been created by manipulating the signaling cascades that regulate the respective developmental stages (Nichols and Smith, 2009; Brons et al., 2007; Tesar, 2005). Remarkably, the principle modes of regulation of these stages are evolutionary conserved, and have led to the derivation of human stem cell lines that represent the lineages of the blastocyst (Thomson et al., 1998; Gafni et al., 2013; Guo et al., 2016; Takashima et al., 2014; Theunissen et al., 2014). Applications of culture conditions that maintain PSCs and the ectopic expression pluripotency transcription factors resulted in derivation of induced (i)PSCs from diverse species of mammals including the human (Takahashi et al., 2007; Takahashi and Yamanaka, 2006; Yu et al., 2007; Liu et al., 2008). This leads to the conclusion that the repertoire of cell types that exist in the blastocyst could be reproduced and maintained indefinitely in culture as stem cell lines. Because regulation of proliferation and differentiation also exist in later stages of embryonic development, this raises the question of whether it is possible to create additional types of non-pluripotent stem cells that represent specific embryonic regions.

One example of post-blastocyst development stage involving extensive proliferation and differentiation is the process of body axis extension. It is initiated from the posterior region of a primitive streak and later continues from the tailbud until the axis extension is completed (Benazeraf and Pourquie, 2013). This process is driven by stem cell-like progenitors that initially form in the primitive streak, and, during the elongation of the axis, reside in the node-streak border (NSB), caudal lateral epiblast (CLE) and the chordoneural hinge (CNH) of the mouse embryo (Henrique et al., 2015). These so-called axial progenitors are thought to gives rise to all peripheral neurons, sclerotome and dermomyotome lineages e.g., motor neurons, peripheral neurons, peripheral nervous system neurons sensory neurons, bone, cartilage, tendon, ligament and/or skeletal muscle cells (Cambray and Wilson, 2002, 2007; Garriock et al., 2015; Attardi et al., 2018) and grafting experiments have indicated that they have a high degree of developmental plasticity (Cambray and Wilson, 2007; Tzouanacou et al., 2009; McGrew et al., 2008).

The specification and maintenance of axial progenitors is driven by interplay of Wnt/β-catenin and FGF signaling (Henrique et al., 2015). Their defining hallmark is co-expression of TBXT and SOX2, which are otherwise generally regarded as non-overlapping key regulators of mesoderm and neural development respectively (Delfino-Machin et al., 2005; Tsakiridis et al., 2014; Gouti et al., 2014; Koch et al., 2017). This expression pattern and their anatomical locations listed above, where they give rise to peripheral neurons and sclerotome and dermomyotome lineages, led their classification as neuromesodermal progenitors (NMPs) (Gouti et al, 2014, Turner et al, 2014 Cambray and Wilson, 2007; Tzouanacou et al., 2009; Brown and Storey, 2000; Wilson et al., 2009; Olivera-Martinez et al., 2012). Notochordal progenitors (Wilson and Beddington, 1996) and anterior and trunk lateral plate mesoderm progenitors (LPMPs) (Kinder et al., 1999; Smith et al., 1994; Taguchi et al., 2014) are additional cell types that exist in the axial regions. Recent global transcriptomics study showed that while all three axial progenitor cell types are clearly distinguishable, they are also share a great deal of gene expression patterns, making them difficult to distinguish in targeted approaches (Wymeersch et al., 2019). To date, the functional developmental potential of axial progenitors and whether they can be converted to stem cell lines are questions that are still open.

Progenitors that mimic the transcriptional phenotypes of axial NMPs including the expression of TBXT and SOX2 have been derived from mouse and human ESCs by activation of Wnt and FGF pathways (Gouti et al., 2014; Turner et al., 2014; Lippmann et al., 2015; Tsakiridis and Wilson, 2015; Gouti et al., 2017; Denham et al., 2015; Verrier et al., 2018). However, their identity, purity, and differentiation potential has been debated (Edri et al., 2018), and, importantly, their long-term self-renewal has not been demonstrated.

Known methods for production of human axial progeny are laborious, costly and resulting transient axial progenitors contaminated by numerous other cell types. Therefore, despite detailed understanding of embryonic axis elongation in mammals, axial stem cells (AxSCs) have not been derived and this stage is inaccessible in human embryos.

### SUMMARY OF THE INVENTION

The present invention relates to a method for producing (or derivation of) axial stem cells (AxSCs) (or neuromuscular-skeletal stem cells), said method comprising: providing: pluripotent stem cells, embryonic or induced pluripotent stem cells (e.g., human PSCs and/or ESC and/or iPSCs, e.g., human ESC line H9 (WA09) or human iPSC line HMGU #1), preferably prior to said derivation/producing said pluripotent cells are maintained in a suitable pluripotent cell media (e.g. mTESR™1, for example, as described in Ludwig T, Thomson J. A., Curr Protoc Stem Cell Biol. 2007 Sep., or StemMACS™ iPS-Brew XF), further preferably said suitable pluripotent cell media is replaced with RPM! 1640 medium supplemented with B27 supplement (e.g., as described in Brewer G J, Cotman C W., Brain Res. 1989 Aug. 7; 494(1):65-74) with or without vitamin A for said derivation/producing (i.e. prior to said derivation/producing); activating the Wnt/β-catenin signalling pathway (e.g., GO:0016055; e.g., Wnt/β-catenin signalling pathway is the series of molecular signals initiated by binding of a Wnt protein to a frizzled family receptor on the surface of the target cell and ending with a change in cell state (Huelsken J, Birchmeier W., New aspects of Wnt signalling pathways in higher vertebrates. Current Opinion in Genetics & Development. 2001 October; 11(5):547-553.); in said pluripotent stem cells, embryonic or induced pluripotent stem cells, preferably said activating is carried out by the means of using an inhibitor of GSK3-β protein (e.g., UniProtKB-P49841), further preferably said inhibitor is CHIR99021, further most preferably said CHIR99021 inhibitor is used at a concentration from about 5 μM to about 10 μM; further most preferably said CHIR99021 inhibitor is used for about 24 hours; passaging the cells derived from the step (b) under the condition of continuous activation the Wnt/β-catenin signalling pathway in said cells during said passaging, preferably said passaging is carried out for at least about 3 to 9 times (e.g., 3, 4, 5, 6, 7, 8, or 9), further preferably said passaging is a serial passaging, further most preferably said passaging comprises re-seeding (e.g., re-seeding is the dissociation of colonies to single cells or clumps of cells and their plating) the cells derived from the step (b) at a lower density into a fresh serum-free medium (e.g., RPMI 1640 medium supplemented with B27 supplement with or without vitamin A); preferably said continuous activation of the Wnt/β-catenin signalling pathway is carried out by the means of using an inhibitor of the Wnt/β-catenin signalling pathway, further preferably said inhibitor is CHIR99021, further most preferably said CHIR99021 inhibitor is used at a concentration of at least about 5 μM (e.g., about 7.5 μM); wherein the cells derived from the step (c) are endogenously expressing transcription factor SOX-2 (e.g., UniProtKB-P48431); optionally, said continuous activating the Wnt/β-catenin signalling pathway from the step (c) is carried out in the presence of: Fibroblast growth factor 2 (e.g., UniProtKB-P09038); and/or a TGF-β (e.g., UniProtKB-P01137) inhibitor; wherein preferably: (d1) said continuous activating the Wnt/β-catenin signalling from the step (c) is carried out with CHIR99021 inhibitor at a concentration of about 5 μM in the presence of: Fibroblast growth factor 2 (e.g., UniProtKB-P09038) and a TGF-β (e.g., UniProtKB-P01137) inhibitor; further preferably said TGF-β inhibitor is SB-431542, further most preferably said SB-431542 inhibitor is used at a concentration of about 10 μM, further further most preferably said Fibroblast growth factor 2 is used at a concentration from about 20 to about 100 ng/ml; wherein the cells derived from the step (d1) are: endogenously expressing: transcription factor SOX-2 (e.g., UniProtKB-P48431), T-box transcription factor T (e.g., UniProtKB-O15178) and Homeobox protein MIXL1 (e.g., UniProtKB-Q9H2W2); not endogenously expressing: paired box protein Pax-6 (e.g., UniProtKB-P26367); optionally, are further endogenously expressing: Homeobox protein CDX-2 (e.g., UniProtKB-Q99626); or (d2) said continuous activating the Wnt/β-catenin signalling from the step (c) is carried out with CHIR99021 inhibitor at a concentration of about 5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM; wherein the cells derived from the step (d2) are: endogenously expressing: transcription factor SOX-2 (e.g., UniProtKB-P48431) and paired box protein Pax-6 (e.g., UniProtKB-P26367); not endogenously expressing: T-box transcription factor T (e.g., UniProtKB-O15178), Homeobox protein MIXL1 (e.g., UniProtKB-Q9H2W2) and Homeobox protein CDX-2 (e.g., UniProtKB-Q99626); or (d3) said continuous activating the Wnt/β-catenin signalling from the step (c) is carried out with CHIR99021 inhibitor at a concentration of about 7.5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM, wherein the cells derived from the step (d3) are: endogenously expressing transcription factor SOX-2 (e.g., UniProtKB-P48431), T-box transcription factor T (e.g., UniProtKB-O15178), Homeobox protein MIXL1 (e.g., UniProtKB-Q9H2W2) and paired box protein Pax-6 (e.g., UniProtKB-P26367); optionally, are further endogenously expressing Homeobox protein CDX-2 (e.g., UniProtKB-Q99626).

The present application satisfies this demand by the provision of methods for producing axial stem cells (AxSCs) as well as axial stem cells (AxSCs), e.g., produced by such methods, and uses thereof described herein below, characterized in the claims and illustrated by the appended Examples and Figures.

Overview of the Sequence Listing

SEQ ID NO 1: is a DNA sequence of GAPDH forward primer (for SYBR Green qPCR).

SEQ ID NO 2: is a DNA sequence of GAPDH reverse primer (for SYBR Green qPCR).

SEQ ID NO 3: is a DNA sequence of MNX1(HB9) forward primer (for SYBR Green qPCR).

SEQ ID NO 4: is a DNA sequence of MNX1(HB9) reverse primer (for SYBR Green qPCR).

SEQ ID NO 5: is a DNA sequence of PRPH forward primer (for SYBR Green qPCR).

SEQ ID NO 6: is a DNA sequence of PRPH reverse primer (for SYBR Green qPCR).

SEQ ID NO 7: is a DNA sequence of POU4F1 forward primer (for SYBR Green qPCR).

SEQ ID NO 8: is a DNA sequence of POU4F1 reverse primer (for SYBR Green qPCR).

SEQ ID NO 9: is a DNA sequence of RUNX2 forward primer (for SYBR Green qPCR).

SEQ ID NO 10: is a DNA sequence of RUNX2 reverse primer (for SYBR Green qPCR).

SEQ ID NO 11: is a DNA sequence of BGLAP forward primer (for SYBR Green qPCR).

SEQ ID NO 12: is a DNA sequence of BGLAP reverse primer (for SYBR Green qPCR).

SEQ ID NO 13: is a DNA sequence of COL1A1 forward primer (for SYBR Green qPCR).

SEQ ID NO 14: is a DNA sequence of COL1A1 reverse primer (for SYBR Green

SEQ ID NO 15: is a DNA sequence of NKX3-2 forward primer (for SYBR Green qPCR).

SEQ ID NO 16: is a DNA sequence of NKX3-2 reverse primer (for SYBR Green qPCR).

SEQ ID NO 17: is a DNA sequence of COMP forward primer (for SYBR Green qPCR).

SEQ ID NO 18: is a DNA sequence of COMP reverse primer (for SYBR Green qPCR).

SEQ ID NO 19: is a DNA sequence of ACAN forward primer (for SYBR Green qPCR).

SEQ ID NO 20: is a DNA sequence of ACAN reverse primer (for SYBR Green qPCR).

SEQ ID NO: 21 is an exemplary amino acid sequence of the human SOX-2 protein, e.g., corresponding to the UniProtKB Accession Number: P48431.

SEQ ID NO: 22 is an exemplary amino acid sequence of the human POU5F1 protein (also known as OCT4), e.g., corresponding to the UniProtKB Accession Number: Q01860-1.

SEQ ID NO: 23 is an exemplary amino acid sequence of the human NANOG protein, e.g., corresponding to the UniProtKB Accession Number: Q9H9S0-1.

SEQ ID NO: 24 is an exemplary amino acid sequence of the human TBXT protein, e.g., corresponding to the UniProtKB Accession Number: O15178-1.

SEQ ID NO: 25 is an exemplary amino acid sequence of the human CDX2 protein, e.g., corresponding to the UniProtKB Accession Number: Q99626-1.

SEQ ID NO: 26 is an exemplary amino acid sequence of the human MIXL1 protein, e.g., corresponding to the UniProtKB Accession Number: Q9H2W2-1.

SEQ ID NO: 27 is an exemplary amino acid sequence of the human PAX6 protein, e.g., corresponding to the UniProtKB Accession Number: P26367-1.

SEQ ID NO: 28 is an exemplary amino acid sequence of the human GSK3B protein, e.g., corresponding to the UniProtKB Accession Number: P49841-1.

SEQ ID NO: 29 is an exemplary amino acid sequence of the human FGF2 protein, e.g., corresponding to the UniProtKB Accession Number: P09038-4.

SEQ ID NO: 30 is an exemplary amino acid sequence of the human TGFB1 protein, e.g., corresponding to the UniProtKB Accession Number: P01137-1.

SEQ ID NO: 31 is an exemplary amino acid sequence of the human LIN28B protein, e.g., corresponding to the UniProtKB Accession Number: Q6ZN17-1.

SEQ ID NO: 32 is an exemplary amino acid sequence of the human MYCN protein, e.g., corresponding to the UniProtKB Accession Number: P04198-1.

SEQ ID NO: 33 is an exemplary amino acid sequence of the human ZIC2 protein, e.g., corresponding to the UniProtKB Accession Number: O95409-1.

SEQ ID NO: 34 is an exemplary amino acid sequence of the human IRX3 protein, e.g., corresponding to the UniProtKB Accession Number: P78415-1.

SEQ ID NO: 35 is an exemplary amino acid sequence of the human SOX1 protein, e.g., corresponding to the UniProtKB Accession Number: O00570-1.

SEQ ID NO: 36 is an exemplary amino acid sequence of the human SOX11 protein, e.g., corresponding to the UniProtKB Accession Number: P35716-1.

SEQ ID NO: 37 is an exemplary amino acid sequence of the human homeobox protein Nkx-2.1, e.g., corresponding to the UniProtKB Accession Number: P43699-1.

SEQ ID NO: 38 is an exemplary amino acid sequence of the human E3 ubiquitin-protein ligase TRIM71, e.g., corresponding to the UniProtKB Accession Number: Q2Q1W2-1.

SEQ ID NO: 39 is an exemplary amino acid sequence of the human forkhead box protein B1, e.g., corresponding to the UniProtKB Accession Number: Q99853-1.

### BRIEF DESCRIPTION OF THE DRAWINGS

FIG. 1: Constitutive Wnt/β-catenin signaling and passaging promotes stable expression of T and SOX2 in hESC progeny. (A) Time course analysis of T by qPCR comparing single and recurrent treatment of hESCs by CHIR99021. (n=3, error bars—SEM, as all other qPCR analyses below). (B-G) Time-course RNA-seq of CHIR99021 constitutively-treated hESCs and over-expressed constitutively active form of β-catenin in hESCs (n=2). (B) Principal component analysis. (C) A panel of primitive streak (T, MIXL1, EVX1, CDX2) node (GSC), paraxial mesoderm (TBX6) marker expression over the time course. (D) A Venn diagram of up-regulated genes at 72 hours compared to untreated cells (FC>2, padj 0.05), and the enriched tissue categories. (E) A heatmap of the up-regulated genes assigned to most enriched tissue categories from (D): ectoderm, mesoderm, neural tube and neural crest. (F) Most significantly enriched signaling cascades in the time course. (G) Fold changes in expression of core pluripotency genes during the time course. (H) Analysis by qPCR of core pluripotency genes and T following 24 hours of 10 μM CHIR99021 treatment and 1, 3 and 5 passages. The passaging medium was supplemented either with 5 μM CHIR99021 (5C) or 5 μM CHIR+100 ng/ml FGF2 (5CF) (n=4).

FIG. 2: WNT and WNT/FGF2 promote establishment of indefinitely renewing axial-like cell lines. (A, B) The treatment scheme used for establishing axial stem cell lines displaying the gradient of CHIR99021 concentrations and presence of FGF2, and TGF-β pathway inhibitor SB431542. Colored sectors mark those conditions that resulted in successful derivation of lines, passage 9 or higher. Full conditions matrix is given in the Figure S2. Based on >3 independent derivations using H9 hESC line. (C) Upper panel, representative phase contrast photomicrographs (10× magnification) of cell lines established by the indicated treatments at passage 25 (inset, a high-density photomicrograph of CHIR99021+FGF2+TGF-β inhibitor SB431542 cell line of the same passage). Bottom panel, representative immunofluorescent photomicrographs (40× magnification) of the corresponding cell lines, passages 26 to 28. SOX2 and T double and CDX2 and PAX6 single staining respectively (scale bar—40 μm). (D,E) qPCR analysis of axial and neural genes of the established cell lines at passages 5, 9 (D) and 26 (E), shown is the fold change relative to undifferentiated H9 hESCs (n=3 independently derived cell lines; NT—not tested, ND—not detected). (F, G) Representative phase contrast photomicrographs (10× magnification) and qPCR analysis (G) of cell lines established in the presence of 7.5 μM CHIR99021 or CHIR99021+ TGF-β inhibitor SB431542 (high-density culture shown in the inset) at passage 25, and the representative immunofluorescent photomicrographs of the CHIR99021+SB431542 sample at passage 28. (G) qPCR analysis of mesoderm and neural genes in the cell lines at passages 5 and 26 (n=3 independently derived cell lines).

FIG. 3: Directed differentiation of axial stem cells. (A) A summary of successfully established cell lines indicating characteristic gene expression patterns and their inferred differentiation bias. (B) Venn diagrams of the up- and down-regulated genes in RNA-sequencing datasets representing cell lines established with CHIR99021 alone, CHIR99021+SB43154 and CHIR99021+SB43154+FGF2 treatments relative to the undifferentiated H9 parental cell line (log2FC>=2, FDR<0.05, passages between 9 and 12). (C) Gene ontology analysis (molecular function) of the significantly up-regulated genes from each cell line. P-value (Fisher test) is shown for every category. (D) qPCR analysis of the indicated genes upon medium swap from ground (CHIR99021+SB43154+FGF2) to primed state (CHIR99021+SB43154) and vice versa for 1, 2 and 3 weeks. Log2 fold change to the undifferentiated parental cell line is shown. ND—Ct value undetermined during qPCR. (E) Predicted differentiating capacity of axial stem cells with major pathways. (F) CHIR+TGFi and CHIR+FGF2+TGFi cells differentiated towards motor neurons for 15 and 28 days. Shown are phase contrast pictures at 10× magnification and single immunofluorescent stainings for Tuj1, Isl1 and HB9 overlaid with DAPI (blue). Magnification—40× for 15 days, 63× for 28 days, scale bars—20 and 50 μm, respectively. (G) qPCR analysis of neuronal markers after 15 and 26 days of differentiation from CHIR+FGF2+TGFi and CHIR+TGFi lines. Log2 fold change to the undifferentiated parental cell line is shown (n=3, NT—not tested). (H) qPCR analysis of osteocytes derived from the indicated conditions after 15 days of differentiation. Log2 fold change to the undifferentiated parental cell line is shown (n=3). (I) qPCR analysis of chondrocytes derived from the indicated conditions after 15 days of differentiation. Log2 fold change to the undifferentiated parental cell line is shown (n=3). (J) Alizarin red staining of CHIR+FGF2+TGFi and CHIR+TGFi lines lines after 15 days of osteogenic differentiation. Undifferentiated CHIR+TGFi control is shown alongside. (K) Alcian blue staining of CHIR+FGF2+TGFi and CHIR+TGFi lines lines after 15 days of chondrogenic differentiation. Undifferentiated CHIR+TGFi control is shown alongside.

FIG. 4: A) Overview of the ligand combinations used in the maintenance medium for axial stem cell derivations. μM in the top row indicate CHIR99021 concentration. Green rectangles indicate successful establishment of cell lines (over 9 passages). Crossed-out rectangles indicate unsuccessful derivation terminated due to cell death, terminal differentiation of lack of proliferation. Gray rectangles indicate cell lines that were maintained to passages over 9, but display unstable proliferation over passages and extended spontaneous differentiation.

FIG. 5: A) Quantitative PCR-based gene expression in 10 individually isolated clones from CHIR-FGF2-SB431542 line displaying heterogeneity in CDX2 expression. Clones were derived from the passage 27 of the parental line and analyzed at the passage 4 following isolation. Gene expression is displayed as relative quantity (to GAPDH). n=1. B) Immunofluorescent analysis of 8 clones from A) for expression of Cdx2 and TBXT. Representative pictures at 63× magnification.

FIG. 6: A) Schematic of axial stem cell derivation from human iPS cell line (HMGU #1). μM concentrations refer to CHIR99021. Concentrations of FGF2 and SB-431542 were identical to derivations from H9 hES cell line—100 ng/ml and 10 μM, respectively. B) Quantitative qPCR analysis of marker gene expression in iPS-derived axial stem cell lines at passage 9. Expression is normalized to GAPDH and plotted as log2 fold change over undifferentiated parental iPS cell line. Error bars represent standard error of the mean between biological replicates (derivations). n=1 to 6.

FIG. 7: A) RNA-seq analysis of Hox gene expression during the time-course CHIR99021 stimulation and β-catenin induction. Time points are 0 (unstimulated), 8, 16, 24, 48 and 72 hours. All differentially expressed (in any time point compared to 0 hours) Hox genes are plotted (fold change >1.5, FDR<0.05). z-score was calculated from normalized counts. B) RNA-seq analysis of Hox gene expression (log 2 fold change) in the established axial stem cell lines. Only differentially expressed (any line over undifferentiated parental hES) Hox genes are plotted (fold change >1.5, FDR<0.05). C) Venn diagram of the overlap in Hox gene expression between 72 hours of CHIR99021 stimulation and the established CHIR-FGF2-SB431542 axial stem cell line (only differentially expressed Hox genes are compared). Genes that are only expressed in the established line are listed on the right.

FIG. 8: Gene expression analysis indicating axial identity of novel indefinitely self-renewing cells. qPCR analysis of pluripotent, axial, neural and mesodermal genes in 24-hour induced cells by CHIR99021 and CHIR99021+FGF2+SB431542 driven cells (CFS) at passage 1, 3 and 5 (fold change relative to undifferentiated H9 hESCs, n=3 independently derived cell lines). (B) qPCR analysis of pluripotent, axial, neural and mesodermal genes in 24-hour induced cells by CHIR99021 and CHIR99021+SB431542 driven cells (CS) at passage 1, 2, 3 and 4 (fold change relative to undifferentiated H9 hESCs, n=3 independently derived cell lines). Error bars represent standard error of the mean (SEM).

FIG. 9: (A) UMAP visualization of single-cell sequencing of CFS and CS lines derived from hESCs (H9 and HUES6) and hiPSCs (HMGU1). (B) Expression of axial markers SOX2, TBXT, CDX2 and neural marker PAX6 at single cell level. (C-J) Dot plots displaying mean expression of lineage-specific markers (C), HOX genes (D), and genes involved in signaling pathways regulated through axial elongation and embryonic development; WNT genes and receptors (E), FGF genes and receptors (F), TGFb genes and receptors (G), NOTCH ligands and receptors (H), retinoic acid receptors (I), BMP genes and receptors (J). Dot size represents percentage of cells expressing respective gene and color intensity indicates expression level.

FIG. 10: (A) UMAP visualization of Louvain clustering in CS lines. (B) Dot plot displaying mean expression of lineage-specific markers in CS clusters. Dot size represents percentage of cells expressing respective gene and color intensity indicates expression level.

FIG. 11: (A) UMAP visualization of Louvain clustering in CS lines. (B) Dot plot displaying mean expression of lineage-specific markers in CS clusters. Dot size represents percentage of cells expressing respective gene and color intensity indicates expression level.

FIG. 12: Neural differentiation of axial stem cells. (A) Schematic representation of neural differentiation process and cytokines in differentiation medium. (B) Representative phase contrast photomicrographs (20× magnification) at DAY2 and DAY16 during differentiation of CFS (upper panel), and CS (bottom panel) lines. (C-D) qPCR analysis of neural markers at DAY28 of differentiation from three CFS (C) and three CS (D) lines (P:parental cells, N:neural differentiation). Each symbol in respective bar displays technical replicates. Error bars represent standard error of the mean (SEM). Fold change is shown as relative to undifferentiated H9 hESCs. (E-F) Immunofluorescence staining of motor neuron marker (MNX1, green), pan-neural marker (TUJ1, red) and DAPI (blue) at DAY28 of differentiation from CFS line (E) and at DAY14 of differentiation from CS line (F) at 63× magnification.

FIG. 13: Skeletal muscle differentiation of axial stem cells. (A) Schematic representation of skeletal muscle differentiation processes (#1-4) and cytokines in differentiation medium. (B-C) Representative phase contrast photomicrographs (20× magnification) at DAY40 of differentiation from CFS lines and at DAY6 differentiation from three independently derived CS lines. Scale bars are 20 μm. (D-G) qPCR analysis of stage-specific muscle development markers at DAY40 of differentiation from CFS lines by using indicated processes (D—#1, E—#2, F—#3, G—#4). Each symbol in respective bar displays technical replicates (P:parental cells, N:neural differentiation). Error bars represent standard error of the mean (SEM). Fold change is shown as relative to undifferentiated H9 hESCs. (H-J) Immunofluorescence staining of muscle markers MyoD (red, H), Myogenin (red, I), M-cadherin (green, H-I), MyHC (red, J) and DAPI (blue, H-J) at DAY40 of differentiation from CFS lines by using indicated processes, scale bar-, magnification-63×.

FIG. 14: Neuromuscular differentiation of CFS line in 3D culture. (A) Schematic representation of 3D differentiation process and cytokines in differentiation medium. (B) Representative phase contrast photomicrograph (5× magnification) of DAY40 organoid differentiated from CFS line. Scale bar is 0.75 mm. (C-D) qPCR analysis of neural markers (C) and muscle markers (D) in DAY40 organoids. (E-F) Whole organoid immunofluorescence staining of motor neuron markers (MNX1, red; OLIG2, purple; ISL1, green), and DAPI (blue) (E) and muscle markers (MyHC, red; ACTA1, green) and DAPI (blue) at DAY40 organoids differentiated from CFS line. Scale bar-100 μM.

FIG. 15: Injection of eGFP-tagged CFS line to chick embryo. (A) Injected eGFP⁺ CFS cells to tail bud region of developing chick embryo at HH17 stage, shown by asterisk. (B) eGFP⁺ CFS cells in chick embryo stopped at HH23-24 stage. (C) Contribution of CFS cells to neural tube (white arrow) and somites (red arrow) in cross section of HH23-24 stage embryo stained with GFP.

FIG. 16: Genes expressed in hAxSCs but not in human NMPs (according to Verrier et al., 2018 Development; Table 51), or mouse NMPs (Gouti et al., Dev Cell. 2017). The figure shows single cell global transcript next generation (all expressed genes) sequencing of human axial stem cells (AxCCs) derived from there sources: Primed: CS_H9: CS state derived from human ES cells line WA09 (H9); Primed: CS_HMGU1: CS state derived from human iPS cells line HMGU1; Primed: CS_HUES6: CS state derived from human ES cells line HUES6; Ground: CFS_H9: CS state derived from human ES cells line WA09 (H9); Ground: CFS_HMGU1: CS state derived from human iPS cells line HMGU1; Ground: CFS_HUES6: CS state derived from human ES cells line HUES6. A. Uniform Manifold Approximation and Projection (UMAP) plot shows the degree of similarity between 28,700 individual cells. B. A panel of genes, namely, MYCN, LIN28B, IRX3, SOX1, ZIC2, SOX11 that are prominently expressed by AxSCs (AxSCs of the ground state (CS on the left) predominantly express MYCN, LIN28B, ZIC2 and SOX11; AxSCs of the primed state (CFS on right) predominantly express MYCN, LIN28B, IRX3, SOX1, ZIC2 and SOX11), but NOT in human and human NMPs derived from human ES cells (Verrier et al., 2018 Development) or Mouse NMPs from embryos (Gouti et al., Dev Cell. 2017).

### DETAILED DESCRIPTION OF THE INVENTION

Despite detailed understanding of embryonic axis elongation in mammals, axial stem cells (AxSCs) have not been derived, and this stage is inaccessible in human embryos. Accordingly, the technical problem underlying the present invention may be formulated as to comply with the needs set out above. The technical problem has been solved by means and methods as described herein and as defined in the claims.

In some aspects, the present invention relates to derivation of axial stem cells from human pluripotent stem cells. In particular, the present invention relates to procedures for establishing region specific multipotent stem cells from pluripotent stem cells, namely axial stem cells. In preferred embodiments, the present invention provides the procedures to derive from human pluripotent stem cells, embryonic and induced pluripotent stem cells (e.g., ESC and iPSCs), daughter stem cells that mimic the characteristics of the precursors that give rise to the axial region, including motor neurons, sensory neurons, bone, cartilage, and skeletal muscle cells.

In preferred embodiments the axial stem cells of the present invention are not pluripotent and hence cannot give rise to teratomas, which is an important concern in cellular therapies. Further preferably, the axial stem cells of the present invention are capable of producing differentiated progeny of higher purity (e.g., cell of the axial region, including motor neurons, sensory neurons, bone, cartilage, and skeletal muscle cells) compared to differentiation of human pluripotent stem cells, which give rise to all cell types of the body. Further most preferably, the axial stem cells of the present invention could drastically shorten and simplify the differentiation protocols for cell types of the axial region, including motor neurons, sensory neurons, bone, cartilage, and skeletal muscle cells.

Advantageously, the methods of the present invention enable, for example, the production of indefinite amount of purer progeny types mentioned above (e.g., cell of the axial region, including motor neurons, sensory neurons, bone, cartilage, and skeletal muscle cells), making the manufacturing feasible, less costly and application of the cells by transplantation safer.

Definitions

As described herein references are made to UniProtKB Accession Numbers (http://www.uniprot.org/, e.g., as available in UniProtKB Release 2020_01 published Feb. 26, 2020).

As described herein references are made to Gene Ontology and GO Annotations database (https://www.ebi.ac.uk/QuickGO/); GO version 2020-04-09; annotation set created on 2020-04-06.

As used herein, the term “Wnt/β-catenin signaling pathway” can be used interchangeably with the term ““Wnt signaling pathway” (e.g., GO:0016055; e.g., Wnt/β-catenin signaling pathway is the series of molecular signals initiated by binding of a Wnt protein to a frizzled family receptor on the surface of the target cell and ending with a change in cell state; Huelsken J, Birchmeier W. New aspects of Wnt signaling pathways in higher vertebrates. Current Opinion in Genetics & Development. 2001 October; 11(5):547-553).

As used herein, the term “axial stem cell/s” (or “AxSC/s”) may interchangeably be used with the term “neuromuscular-skeletal stem cells” and may refer to cells produced by the methods of the present invention or having characteristics of the cells produced by the methods of the present invention as described herein below.

As used herein, the term “pluripotent stem cells” (or “PSCs”) may refer to cells capable of differentiating into cells of any type of tissue, e.g., which could be obtained by means other than the destruction of an embryo (e.g., human embryo) (e.g., Yu et al., 2007, Science. 2007 Dec. 21; 318(5858):1917-20).

As used herein, the term “embryonic stem cells” (or “ESCs”) may refer to embryonic pluripotent stem cells that may be produced without destroying an embryo (e.g., human embryo) (e.g., Chung et al. Cell Stem Cell, Feb. 2008, Vol. 2, pages 113-117).

As used herein, the term “induced pluripotent stem cells” (or “iPSCs”) may refer to pluripotent stem cells that can be generated directly from adult cells (e.g., Yu et al., 2007), i.e., they are obtainable without destruction of embryos, e.g., they are obtainable from somatic cells (e.g., fibroblasts).

As used herein, the term “neuromesodermal progenitors” (or NMps) may refer to embryonic cell-type that contributes to the development of both the spinal cord and paraxial mesoderm (e.g., Tzouanacou et al., 2009). However, NMPs are neither capable of indefinitely renewing themselves nor capable of differentiating themselves into different stem cell sub-types (e.g., CFS and CS as used herein). Furthermore, NMPs are transient cells, meaning they can not be propagated (e.g., more than 20 passages). Additionally, NMPs express a different repertoire of genes compared to the axial stem cells of the present invention. For examples, NMPs are not expressing MYCN, LIN28B, IRX3, SOX1, ZIC2, SOX11 proteins as defined herein.

As used herein, the term “induced neural stem cells” (or iNSCs) may refer to somatically-derived neural stem cells that are self-renewing, multipotent cells capable of differentiating into neurons, astrocytes and oligodendrocytes.

As used herein, the term “substantially not expressing” may refer to an expression level that is less than 10% (e.g., less than 7%, less than 4%, less than 1%, less than 0.5%, or less than 0.1%) of the expression level of the control (e.g., starting cell, e.g., undifferentiated cell, e.g., undifferentiated H9 hESCs).

In the course of the present invention it was found that serial passaging of human ESCs and iPSCs when TXBT/BRA and SOX2 are endogenously induced enables to establish two states of indefinitely self-renewing morphologically compact stem cell lines. Establishment under WNT/FGF produced SOX2/TBXT lines that resemble neuromesodermal progenitors, while WNT alone gave rise to SOX2/PAX6 lines that could also be derived from the first state, but not vice versa. Inhibition of TGF-β improved the derivation process, clonal passaging, and reduced spontaneous differentiation. Importantly, the two states readily differentiated into peripheral neurons neuronal types with peripheral and motor characteristics, and to sclerotome and dermomyotomesomitic mesoderm, and, importantly, but they are functionally similar, differentiate rapidly and uniformly to motor neurons, and did not form teratomas. Hence, ground and primed state human AxSCs could be derived from pluripotent cells, and have potential to become a basis for treatments of peripheral nerve degeneration and injury, and a benchmark for research ofon neurodegeneration, axial development, and evolution.

In the course of the present invention processes were developed for creating human stem cell lines that fulfill known criteria of axial progenitors. The derivation was, for example, based on human ESCs and iPSCs because it is not permitted to manipulate human embryos after implantation and beginning of gastrulation. Conditions were initially defined that induce the endogenous expression of TBXT and SOX2 in progeny of hPSCs because these genes are some of the earliest markers that characterize axial progenitors in the NSB, CLE and CNH. To create cell lines serial passaging was used because it was surprisingly found that this maintained the expression of SOX2, a transcription factor that is crucial for self-renewal of pluripotent and neural stem cells (NSCs). Wnt/β-catenin and FGF inducers and TGF-β inhibitor were studied because these pathways were activated in cells that up-regulated TBXT. The establishment of cell lines was apparent, e.g., within three to five passages, and the levels of the key developmental genes remained stable for dozens of passages. Moreover, the use of TGF-β inhibitor minimized the spontaneous differentiation of the cell lines, and enabled us to create single cell clones. Remarkably, two developmental states were captured by these cell lines, which maintained SOX2 at the level of PSCs, but were mutually exclusive for expression of PAX6 and TBXT in the presence of FGF2. In the course of the present invention it was possible to differentiate newly established axial cell lines to peripheral neurons and mesodermal sclerotome and dermomyotome progeny in vitro. To test their teratoma formation capabilities, all cell lines were injected to immunodeficient mice, which showed no signs of such tumours. According to the impact of previous derivations of stem cell lines, such as ESCs and NSCs, it is contemplated that the representation of human axial progenitors in culture in the form of indefinitely renewing stem cell lines could spark novel basic and medical research avenues. This may, e.g., include the regulation of axial development, neuromuscular diseases and evolution, and novel applications in the treatment of peripheral nervous system diseases and trauma by the development of drug compounds and cell therapies.

The axial stem cells (AxSCs) of the present invention are not neuromesodermal progenitors (NMPs). Thus, it is known from the prior art that NMPs are transient cells, meaning that they can not be propagated, whereas the axial stem cells (AxSCs) of the present invention are not transient and can be propagated (e.g., more than 20 passages).

The embodiments which characterize the present invention are described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

In another embodiment the present invention relates to a method for producing (or derivation of) axial stem cells (AxSCs) (or neuromuscular-skeletal stem cells), said method comprising: providing: pluripotent stem cells, embryonic or induced pluripotent stem cells (e.g., human PSCs and/or ESC and/or iPSCs, e.g., human ESC line H9 (WA09) or human iPSC line HMGU #1), preferably prior to said derivation/producing said pluripotent cells are maintained in a suitable pluripotent cell media (e.g., mTESR1 or iPS-Brew etc.), further preferably said suitable pluripotent cell media is replaced with RPMI 1640 medium supplemented with B27 supplement with or without vitamin A for said derivation/producing (i.e. prior to said derivation/producing); activating the Wnt/β-catenin signaling pathway (e.g., GO:0016055; e.g., Wnt/β-catenin signaling pathway is the series of molecular signals initiated by binding of a Wnt protein to a frizzled family receptor on the surface of the target cell and ending with a change in cell state (Huelsken J, Birchmeier W. New aspects of Wnt signaling pathways in higher vertebrates. Current Opinion in Genetics & Development. 2001 Octtober; 11(5):547-553.); in said pluripotent stem cells, embryonic or induced pluripotent stem cells, preferably said activating is carried out by the means of using an inhibitor of GSK3b protein (e.g., UniProtKB-P49841), further preferably said inhibitor is CHIR99021, further most preferably said CHIR99021 inhibitor is used at a concentration from about 5 μM to about 10 μM; further most preferably said CHIR99021 inhibitor is used for about 24 hours; passaging the cells derived from the step (b) under the condition of continuous activation the Wnt/β-catenin signaling pathway in said cells during said passaging, preferably said passaging is carried out for at least about 3 to 9 times (e.g., 3, 4, 5, 6, 7, 8, or 9), further preferably said passaging is a serial passaging, further most preferably said passaging comprises re-seeding (e.g., re-seeding is the dissociation of colonies to single cells or clumps of cells and their plating) the cells derived from the step (b) at a lower density into a fresh serum-free medium (e.g., RPMI 1640 medium supplemented with B27 supplement with or without vitamin A); preferably said continuous activation of the Wnt/β-catenin signaling pathway is carried out by the means of using an inhibitor of the Wnt/β-catenin signaling pathway, further preferably said inhibitor is CHIR99021, further most preferably said CHIR99021 inhibitor is used at a concentration of at least about 5 μM (e.g., about 7.5 μM); wherein the cells derived from the step (c) are endogenously expressing transcription factor SOX-2 (e.g., UniProtKB-P48431); wherein optionally:

(d) said continuous activating the Wnt/β-catenin signaling pathway from the step (c) is carried out in the presence of: Fibroblast growth factor 2 (e.g., UniProtKB-P09038); and/or a TGF-β (e.g., UniProtKB-P01137) inhibitor; wherein preferably:

(d1) said continuous activating the Wnt/β-catenin signaling from the step (c) is carried out with CHIR99021 inhibitor at a concentration of about 5 μM in the presence of: Fibroblast growth factor 2 (e.g., UniProtKB-P09038) and a TGF-β (e.g., UniProtKB-P01137) inhibitor; further preferably said TGF-β inhibitor is SB-431542, further most preferably said SB-431542 inhibitor is used at a concentration of about 10 μM, further further most preferably said Fibroblast growth factor 2 is used at a concentration from about 20 to about 100 ng/ml; wherein the cells derived from the step (d1) are: endogenously expressing: transcription factor SOX-2 (e.g., UniProtKB-P48431), T-box transcription factor T (e.g., UniProtKB-O15178) and Homeobox protein MIXL1 (e.g., UniProtKB-Q9H2W2); not endogenously expressing: paired box protein Pax-6 (e.g., UniProtKB-P26367); optionally, are further endogenously expressing: Homeobox protein CDX-2 (e.g., UniProtKB-Q99626); or

(d2) said continuous activating the Wnt/β-catenin signaling from the step (c) is carried out with CHIR99021 inhibitor at a concentration of about 5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM; wherein the cells derived from the step (d2) are: endogenously expressing: transcription factor SOX-2 (e.g., UniProtKB-P48431) and paired box protein Pax-6 (e.g., UniProtKB-P26367); not endogenously expressing: T-box transcription factor T (e.g., UniProtKB-O15178), Homeobox protein MIXL1 (e.g., UniProtKB-Q9H2W2) and Homeobox protein CDX-2 (e.g., UniProtKB-Q99626); or

(d3) said continuous activating the Wnt/β-catenin signaling from the step (c) is carried out with CHIR99021 inhibitor at a concentration of about 7.5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM, wherein the cells derived from the step (d3) are: endogenously expressing transcription factor SOX-2 (e.g., UniProtKB-P48431), T-box transcription factor T (e.g., UniProtKB-O15178), Homeobox protein MIXL1 (e.g., UniProtKB-Q9H2W2) and paired box protein Pax-6 (e.g., UniProtKB-P26367); optionally, are further endogenously expressing Homeobox protein CDX-2 (e.g., UniProtKB-Q99626).

Exemplary RPMI-1640 Medium Composition:

(e.g., as described at https://www.sigmaaldrich.com/life-science/cell-culture/learning-center/media-formulations/rpmi-1640.html)

| | |
|---|---|
| Component | g/L |

Inorganic Salts

| | |
|---|---|
| Calcium Nitrate•4H₂O | 0.1 |
| Magnesium Sulfate (anhydrous) | 0.04884 |
| Potassium Chloride | 0.4 |
| Sodium Bicarbonate | — |
| Sodium Chloride | 6 |
| Sodium Phosphate Dibasic (anhydrous) | 0.8 |

Amino Acids

| | |
|---|---|
| L-Alanyl-L-Glutamine | — |
| L-Arginine | 0.2 |
| L-Asparagine (anhydrous) | 0.05 |
| L-Aspartic Acid | 0.02 |
| L-Cystine•2HCl | 0.0652 |
| L-Glutamic Acid | 0.02 |
| L-Glutamine | 0.3 |
| Glycine | 0.01 |
| L-Histidine | 0.015 |
| Hydroxy-L-Proline | 0.02 |
| L-Isoleucine | 0.05 |
| L-Leucine | 0.05 |
| L-Lysine•HCl | 0.04 |
| L-Methionine | 0.015 |
| L-Phenylalanine | 0.015 |
| L-Proline | 0.02 |
| L-Serine | 0.03 |
| L-Threonine | 0.02 |
| L-Tryptophan | 0.005 |
| L-Tyrosine•2Na•2H₂O | 0.02883 |
| L-Valine | 0.02 |

Vitamins

| | |
|---|---|
| D-Biotin | 0.0002 |
| Choline Chloride | 0.003 |
| Folic Acid | 0.001 |
| myo-Inositol | 0.035 |
| Niacinamide | 0.001 |
| p-Aminobenzoic Acid | 0.001 |
| D-Pantothenic Acid (hemicalcium) | 0.00025 |
| Pyridoxine•HCl | 0.001 |
| Riboflavin | 0.0002 |
| Thiamine•HCl | 0.001 |
| Vitamin B₁₂ | 0.000005 |

Other

| | |
|---|---|
| D-Glucose | — |
| Glutathione (reduced) | 0.001 |
| Phenol Red•Na | 0.0053 |

Add

| | |
|---|---|
| L-Glutamine | — |
| Sodium Bicarbonate | 2 |

Exemplary B27 composition and preparation protocol (e.g., as described by Hanna LabProtocol—Weizmann Institute of Science, e.g., available at https://hannalabweb.weizmann.ac.il/):

Making stocks for future reuse for some of the components:

3) Human Insulin (Sigma or PROSPEC BIO CYT) (125 mg is needed for 800 ml B27). Prepare a 25 mg/ml stock solution by dissolving 250 mg insulin in 10 ml 0.005 M HCl overnight or even 2 days at 4° C. Store in 1 ml aliquots in −80° C. (use 5 vials per 800 ml B27).

6) T3 (Sigma) (80 μg is needed for 800 ml B27). Prepare a 2 mg/ml stock solution by dissolving 100 mg T3 in 1 ml DMSO and then in 49 ml of Ethanol. Store in 40 μl individual aliquots at −80° C. (use 1 vial per 800 ml B27 supplement).

11) Sodium Selenite (Sigma, 1mg) (500 μg is needed for 800 ml B27). Prepare a 1 mg/ml stock by dissolving the bottle in 1 ml dH2O. Add 500 μl per 800 ml B27 supplement).

12) Corticosterone (Sigma, 1g) (800 μg is needed for 800 ml B27). Prepare a 2 mg/ml stock by dissolving 0.1 g Corticosterone into 50 ml Ethanol. Make 400 μl individual aliquots (use 1 vial per 800 ml B27). Store at −80° C.

13) Linoleic acid (Sigma, 100 MG) (40 mg is needed for 800 ml B27). Prepare a 100 mg/ml stock by adding 0.9 ml of Ethanol. Make 400 μl individual aliquots (use 1 vial per 800 ml B27). Store at −80° C. .

14) Linolenic acid (Sigma, 500 MG) (40mg is needed for 800 ml B27). Prepare a 100 mg/ml stock by adding 4.5 ml of Ethanol. Make 400 μl individual aliquots (use 1 vial per 800 ml B27). Store at −80° C.

15) Progesterone (Sigma, 1 g) (0.252 mg is needed per 800 ml B27 stock). Prepare a 1 mg/ml stock by dissolving 10 mg Progesterone into 10 ml Ethanol. Store at −80° C. Make 252 μl individual aliquots (use 1 vialper 800 ml B27)

16) Retinol acetate (Sigma, 1 g) (4 mg is needed per 800 ml B27 stock). Prepare a 20 mg/ml stock dissolving the bottle in 50 ml of Ethanol. Make 200 μl individual aliquots (use 1 vial per 800 ml B27). Store at −80° C.

17) DL-alpha tocopherol (vit E) (Sigma, 5 G) (40 mg is needed per 800 ml B27 stock). Prepare a 100 mg/ml stock by dissolving the bottle in 45 ml Ethanol. Make 400 μl individual aliquots (use 1 vial per 800 ml B27). Store at −80° C.

18) DL-alpha tocopherol acetate (Sigma 10 G) (40 mg is needed per 800 ml B27). Prepare a 100 mg/ml stock by dissolving the bottle in 90 ml Ethanol. Make 400 μl individual aliquots(use 1 vial per 800 ml B27). Store at −80C.

19) Oleic acid (Sigma 1 G) (40 mg is needed per 800 ml B27 stock). Prepare a 100 mg/ml stock by adding 9 ml of Ethanol. Make 400μl individual aliquots (use 1 vial per 800 ml B27). Store at −80C.

20) Pipecolic acid (Sigma, 100 MG) (40 mg is needed per 800 ml B27 stock). Prepare a 50 mg/ml stock by adding 2 ml of water. Make 800 μl individual aliquots (use 1 vial per 800 ml B27). Store at −80C.

For a total of 800 ml of B27 supplement assemble the following 22 ingredients (e.g., from Sigma-Aldrich) in a 500 ml of Neurobasal medium as a base (Invitrogen):

1) In an empty and sterile 1 L glass bottle insert total of 100 gr of BSA Fraction V IgG free Fatty Acid Poor powder (Invitrogen) and 500 ml of Neurobasal medium. (The remaining 300 ml of Neurobasal are used for dissolving several of the components indicated below).

2) Dissolve Biotin (1 unit of 100 mg, e.g., from Sigma) in 10 ml of Neurobasal media, and add to mix.

3) Dissolve Catalase (1 unit of 100 mg, e.g., Sigma) in 10 ml of Neurobasal media, and add to mix.

4) Dissolve all 4 units of Superoxide Dismutase in 10 ml of Neurobasal medium and add to mix.

5) Weigh 40 mg of Glutathione and directly add to mix.

6) Dissolve all 2 units of Holo-Transferinin 10 ml of Neurobasal medium and add to mix.

7) Weigh 80 mg of L-carnitineand and directly add to mix.

8) Weigh 600 mg of D-galactoseand and directly add to mix.

9) Weigh 644 mg of Putrescine and directly add to mix.

10) Add 40 μL of Ethanolamine and directly to mix.

11) Add 1 vial of Progesterone stock (dissolved in Ethanol and frozen at −80), directly to mix. Progesterone stock preparation: prepare a 1 mg/ml stock by dissolving 10 mg Progesterone in 10 ml Ethanol. Store at −80C. Make 252 μl individual aliquots (use 1 vial per 800 ml B27).

12) Add 5 vials of Insulin stock (dissolved and frozen at −80) directly to mix.

13) Add 1 vial of T3 (dissolved in Ethanol and frozen at −80) and directly add to mix.

14) Add 1 vial of Pipecolic Acid stock (dissolved in water and frozen at −80), directly to mix.

15) Add 1 vial of Oleic Acid stock (dissolved in Ethanol and frozen at −80), directly to mix.

16) Add 1 vial of Linoleic Acid stock (dissolved in Ethanol and frozen at −80), directly to mix.

17) Add 1 vial of Linolenic Acid stock (dissolved in Ethanol and frozen at −80), directly to mix.

18) Add 1 vial of Retinol acetatestock (dissolved in Ethanol and frozen at −80), directly to mix. For the preparation of the Vitamin A free B27 supplement: Retinol acetate is removed from the composition.

19) Add 1 vial of DL-alpha tocopherol (vit. E) stock (dissolved in Ethanol and frozen at −80), directly to mix.

20) Add 1 vial of DL-alpha tocopherol acetate stock (dissolved in Ethanol and frozen at −80), directly to mix.

21) Add 1 vial of Corticosterone stock (dissolved in Ethanol and frozen at −80), directly to mix.

22) Add 1 vial of Sodium Selenite stock (dissolved in Ethanol and frozen at −80), directly to mix.

23) Add whatever is remaining (leftover) of the 300 ml of Neurobasal. Gently mix the bottle (no need for pipetting or harsh shaking). 10 times gentle rocking. Leave bottle 12 h (overnight) at 4 C for optimal dissolving (without any shaking, and keep protected from light). Next day, make 5 ml Aliquots and store at −20 protected from light. (Stable for 1 year at −20). Avoid repeated Freezing and thawing. Mix is too viscous for filtering, but can be filtered upon addition to media later on: for Mouse PSCs 5 ml B27 per 500 ml media bottle(1 aliquot) can be used. For Human PSCs: 10 ml B27 per 500 ml media bottle (2 aliquots) can be used.

In a further embodiment, the present invention relates to a method of the present invention, wherein the continuous activating the Wnt/β-catenin signaling pathway from the step (c) is carried out in the presence of: Fibroblast growth factor 2 (e.g., UniProtKB-P09038); and/or a TGF-β (e.g., UniProtKB-P01137) inhibitor.

In a further embodiment, the present invention relates to a method of the present invention, wherein the continuous activating the Wnt/β-catenin signaling from the step (c) is carried out with CHIR99021 inhibitor at a concentration of about 5 μM in the presence of: Fibroblast growth factor 2 (e.g., UniProtKB-P09038) and a TGF-β (e.g., UniProtKB-P01137) inhibitor; further preferably said TGF-β inhibitor is SB-431542, further most preferably said SB-431542 inhibitor is used at a concentration of about 10 μM, further further most preferably said Fibroblast growth factor 2 is used at a concentration from about 20 to about 100 ng/ml; wherein the cells derived from the step (d1) are: endogenously expressing: transcription factor SOX-2 (e.g., UniProtKB-P48431), T-box transcription factor T (e.g., UniProtKB-O15178) and Homeobox protein MIXL1 (e.g., UniProtKB-Q9H2W2); not endogenously expressing: paired box protein Pax-6 (e.g., UniProtKB-P26367); optionally, are further endogenously expressing: Homeobox protein CDX-2 (e.g., UniProtKB-Q99626).

In a further embodiment, the present invention relates to a method of the present invention, wherein the continuous activating the Wnt/β-catenin signaling from the step (c) is carried out with CHIR99021 inhibitor at a concentration of about 5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM; wherein the cells derived from the step (d2) are: endogenously expressing: transcription factor SOX-2 (e.g., UniProtKB-P48431) and paired box protein Pax-6 (e.g., UniProtKB-P26367); not endogenously expressing: T-box transcription factor T (e.g., UniProtKB-O15178), Homeobox protein MIXL1 (e.g., UniProtKB-Q9H2W2) and Homeobox protein CDX-2 (e.g., UniProtKB-Q99626).

In a further embodiment, the present invention relates to a method of the present invention, wherein the continuous activating the Wnt/β-catenin signaling from the step (c) is carried out with CHIR99021 inhibitor at a concentration of about 7.5 μM, preferably in the presence of a TGF-β (e.g., UniProtKB-P01137) inhibitor, further preferably said TGF-β inhibitor is SB-431542, further most preferably said SB-431542 inhibitor is used at a concentration of about 10 μM, wherein the cells derived from the step (d3) are: endogenously expressing transcription factor SOX-2 (e.g., UniProtKB-P48431), T-box transcription factor T (e.g., UniProtKB-O15178), Homeobox protein MIXL1 (e.g., UniProtKB-Q9H2W2) and paired box protein Pax-6 (e.g., UniProtKB-P26367); optionally, are further endogenously expressing Homeobox protein CDX-2 (e.g., UniProtKB-Q99626).

In a further embodiment, the present invention relates to a method of the present invention, comprising step (d).

In a further embodiment, the present invention relates to a method of the present invention, comprising step (d); and further comprising: step (d1), (d2) or (d3).

In a further embodiment, the present invention relates to a method of the present invention, further comprising passaging the cells derived from the step (c) and/or step (d).

In a further embodiment, the present invention relates to a method of the present invention, wherein the axial stem cells of the present invention have one or more of the following characteristics: expressing transcription factor SOX-2 (e.g., UniProtKB-P48431); substantially not expressing OCT4 transcription factor (e.g., UniProtKB-Q01860); substantially not expressing homeobox protein NANOG (e.g., UniProtKB-Q9H9S0); are not pluripotent; are a region-specific multipotent stem cells; are obtainable from a pluripotent stem cell, embryonic or induced pluripotent stem cell (e.g., human PSCs and/or ESC and/or iPSCs, e.g., human ESC line H9 (WA09) or human iPSC line HMGU #1); are not capable of differentiating into cell types of all tissues of the embryo; are only capable of differentiating in cell types emerging from the region of the central body axis during embryonic development (e.g., sclerotome, dermomyotome and peripheral neurons); are not capable to form teratomas; are capable of mimicking the characteristics of the precursors that give rise to the axial region (e.g., motor neurons, peripheral neurons, peripheral nervous system neurons sensory neurons, bone, cartilage, tendon, ligament and/or skeletal muscle cells); are not transient cells; are capable of differentiating into a motor neuron, peripheral neuron, muscle, cartilage or bone progenitor; are indefinitely renewing stem cells; are capable to grow as clones; are not neuromesodermal progenitors (NMps) and/or is not induced neural stem cells (iNSCs).

In a further embodiment, the present invention relates to a method of the present invention, wherein the axial stem cells of the present invention are capable of indefinitely renewing and differentiating, for example, into: (a) ground axial stem cells (e.g., indefinitely renewing ground axial stem cells, may also be referred to herein as “CFS”), wherein said ground axial stem cell, expressing: transcription factor SOX-2 (e.g., UniProtKB-P48431); T-box transcription factor T (e.g., UniProtKB-O15178); homeobox protein CDX-2 (e.g., UniProtKB-Q99626); and homeobox protein MIXL1 (e.g., UniProtKB-Q9H2W2); and/or (b) primed state axial stem cells (e.g., indefinitely renewing primed state axial stem cells, may also be referred to herein as “CS”), wherein said primed axial stem cell, expressing: transcription factor SOX-2 (e.g., UniProtKB-P48431) and paired box protein PAX-6 (e.g., UniProtKB-P26367).

In a further embodiment, the present invention relates to a method of the present invention, wherein the axial stem cells are human axial stem cells.

In a further embodiment, the present invention relates to an axial stem cell/s produced by the method of the present invention.

In a further embodiment, the present invention relates to an axial stem cell/s (AxSC/s) (e.g., isolated AxSC/s), wherein said axial stem cell has one or more of the following characteristics: expressing the transcription factor SOX-2 (e.g., UniProtKB-P48431), substantially not expressing OCT4 transcription factor (e.g., UniProtKB-Q01860); substantially not expressing homeobox protein NANOG (e.g., UniProtKB-Q9H9S0), said AxSC is not pluripotent, is a region-specific multipotent stem cell; is obtainable from a pluripotent stem cell, embryonic or induced pluripotent stem cell (e.g., ESC and/or iPSCs, e.g., human ESC line H9 (WA09) or human iPSC line HMGU #1); is not capable of differentiating into cell types of all tissues of the embryo; is only capable of differentiating in cell types emerging from the region of the central body axis during embryonic development (e.g., sclerotome, dermomyotome and peripheral neurons); is not capable to form teratomas; is capable of mimicking the characteristics of the precursors that give rise to the axial region (e.g., motor neurons, peripheral neurons, peripheral nervous system neurons sensory neurons, bone, cartilage, tendon, ligament and/or skeletal muscle cells); is not a transient cell; is capable of differentiating into a motor neuron, peripheral neuron, muscle, cartilage or bone progenitor; is an indefinitely renewing stem cell; is capable to grow as clones; is not a neuromesodermal progenitor (NMp); is not an induced neural stem cell (iNSC).

In a further embodiment, the present invention relates to an axial stem cell of the present invention, wherein said AxSC is capable of indefinitely renewing itself and, for example, differentiating into: (a) a ground axial stem cell (e.g., indefinitely renewing ground state axial stem cell, may also be referred to herein as “CFS”), wherein said ground axial stem cell, expressing: transcription factor SOX-2 (e.g., UniProtKB-P48431); T-box transcription factor T (e.g., UniProtKB-O15178); homeobox protein CDX-2 (e.g., UniProtKB-Q99626); and homeobox protein MIXL1 (e.g., UniProtKB-Q9H2W2); and/or (b) primed state axial stem cell (e.g., indefinitely renewing primed state axial stem cell, may also be referred to herein as “CS”), wherein said primed axial stem cell, expressing: transcription factor SOX-2 (e.g., UniProtKB-P48431) and paired box protein PAX-6 (e.g., UniProtKB-P26367).

In a further embodiment, the present invention relates to an axial stem cell of the present invention, wherein said AxSC is endogenously expressing: transcription factor SOX-2 (e.g., UniProtKB-P48431), T-box transcription factor T (e.g., UniProtKB-O15178) and Homeobox protein MIXL1 (e.g., UniProtKB-Q9H2W2); not endogenously expressing: paired box protein Pax-6 (e.g., UniProtKB-P26367); optionally, are further endogenously expressing: Homeobox protein CDX-2 (e.g., UniProtKB-Q99626).

In a further embodiment, the present invention relates to an axial stem cell of the present invention, wherein said AxSC is endogenously expressing: transcription factor SOX-2 (e.g., UniProtKB-P48431) and paired box protein Pax-6 (e.g., UniProtKB-P26367); not endogenously expressing: T-box transcription factor T (e.g., UniProtKB-O15178), Homeobox protein MIXL1 (e.g., UniProtKB-Q9H2W2) and Homeobox protein CDX-2 (e.g., UniProtKB-Q99626).

In a further embodiment, the present invention relates to an axial stem cell of the present invention, wherein said AxSC is endogenously expressing transcription factor SOX-2 (e.g., UniProtKB-P48431), T-box transcription factor T (e.g., UniProtKB-O15178), Homeobox protein MIXL1 (e.g., UniProtKB-Q9H2W2) and paired box protein Pax-6 (e.g., UniProtKB-P26367); optionally, are further endogenously expressing Homeobox protein CDX-2 (e.g., UniProtKB-Q99626).

In a further embodiment, the present invention relates to an axial stem cell of the present invention, wherein said AxSC is a human axial stem cell (e.g., isolated).

In a further embodiment, the present invention relates to a composition, preparation and/or kit comprising the axial stem cell of the present invention.

In a further embodiment, the composition, preparation and/or kit of the present invention is a pharmaceutical and/or diagnostic composition, preparation or kit.

In a further embodiment, the axial stem cell/s, composition, preparation and/or kit of the present invention is used as a medicament.

In a further embodiment, the axial stem cell/s, composition, preparation and/or kit of the present invention is used in one or more of the following methods: in a method of treatment, amelioration, prophylaxis and/or diagnostics of a neurodegenerative disease; in a method of treatment, amelioration, prophylaxis and/or diagnostics of a bone and/or cartilage disorder; in a method of treatment, amelioration, prophylaxis and/or diagnostics of muscle disorder; in a method of regenerative treatment of a cell, tissue, organ and/or body; in a method for screening a candidate compound for activity against a disease (e.g., a disease of the peripheral nervous system or a disease relating to axial stem cells, e.g. muscle-, motor neuron-, peripheral neuron, sensory neuron cartilage-, tendon-related (e.g., degenerative) disease) and/or for neurotoxicity screening; in a method for treatment, amelioration, prophylaxis and/or diagnostics of a disease relating to axial stem cells, e.g. muscle-, motor neuron-, peripheral neuron-, sensory neuron cartilage-, tendon-related (e.g., degenerative) disease; in any of preceding methods, wherein said method is an in vitro, ex vivo or in vivo method.

In a further embodiment, the present invention relates to a method for/of improving the condition of a sample and/or subject, e.g., in need thereof, preferably wherein said method is one or more of the following: a method of treatment, amelioration, prophylaxis and/or diagnostics of a neurodegenerative disease; a method of treatment, amelioration, prophylaxis and/or diagnostics of a bone and/or cartilage disorder; a method of treatment, amelioration, prophylaxis and/or diagnostics of muscle disorder; a method of regenerative treatment of a cell, tissue, organ and/or body; a method for screening a candidate compound for activity against a disease (e.g., a disease of the peripheral nervous system) and/or for neurotoxicity screening; said method comprising: providing the axial stem cell, composition, preparation and/or kit of the present invention; to said sample and/or subject; and administering, e.g., a therapeutically effective amount of said the axial stem cell, composition, preparation and/or kit; to said sample and/or subject.

In a further embodiment, the present invention relates to the method of the present invention, wherein said method is an in vitro, ex vivo or in vivo method.

In a further embodiment, the present invention relates to use of the axial stem cell, composition, preparation and/or kit of the present invention, for example, for one or more of the following: for treatment, amelioration, prophylaxis and/or diagnostics of a neurodegenerative disease; for treatment, amelioration, prophylaxis and/or diagnostics of a bone and/or cartilage disorder; for treatment, amelioration, prophylaxis and/or diagnostics of muscle disorder; for regenerative treatment of a cell, tissue, organ and/or body; for screening a candidate compound for activity against a disease (e.g., a disease of the peripheral nervous system) and/or for neurotoxicity screening; for any one of preceding uses, wherein said use is an in vitro, ex vivo or in vivo use.

In a further embodiment, the present invention relates to the use according to the present invention, wherein said use is an in vitro, ex vivo or in vivo use or combination thereof.

In a preferred embodiment, the methods of the present invention are carried out with following starting cells (i.e., cells provided in the methods of the present invention): starting cells are pluripotent cells; they can be either embryonic stem cells (ESC) or induced pluripotent stem cells (iPSC), preferably starting cells are pluripotent cells of human origin (however, the present invention also includes pluripotent cells of non-human origin, such as primate cells or mouse); pluripotent cells are described in the literature and are, e.g., characterized by the co-expression of classical pluripotency factors POU5F1 (OCT4), NANOG, SOX2 as well as the ability to be indefinitely self-renew in cell culture and ability to differentiate towards endodermal, mesodermal, ectodermal and extraembryonic tissues types.

In a further preferred embodiment, the methods of the present invention comprise the following derivation steps applied to the starting cells to obtain final cells (axial stem cells): the first and second steps as described herein below.

The first step in derivation of multipotent axial stem cells is the induction of early primitive streak-like state in pluripotent cells (this step is short, e.g., 24 hours, and ensures that the cells are given an impulse to differentiate towards primitive streak progenitors; preferably, this step is not long enough to fully downregulate pluripotency genes POU5F1, NANOG and/or SOX2; further preferably, this step is carried out on cells growing at high density, e.g., in 2D monolayer in a defined serum-free medium of the present invention.

The second step is the actual generation of multipotent axial stem cells. The rationale for this step is to maintain in the primitive-like cells generated in the step above a) ability of to self-renew and b) characteristics of multipotent progenitors contributing towards postoccipital axis elongation in mammals. This step is carried out by, e.g., re-seeding the induced cells at a much lower density into a defined serum-free medium with certain ligands. Successful generation of candidate stem cells lines can be visually manifested in the formation of small tight colonies that can be further propagated in the same (corresponding) media. The axial stem cell lines undergo an empirical establishment phase of, e.g., up to 9 passages (e.g., 3 to 9 passages). During this time, marker gene expression can be monitored for consistency. After the establishment phase, the cells are considered “derived” and can be used for further differentiation into terminal somatic cell types: a) spinal motor neurons and b) sclerotome derivatives (chondrocytes and osteocytes). These cells can also be converted to dermomyotome derivatives—skeletal muscle cells and adipocytes.

In a further preferred embodiment, the methods of the present invention use the following media composition and/or ligands: for the initial induction step, cells can be transferred to a suitable medium, e.g., composed of RPM11640 supplemented with B27 supplement with or without vitamin A; CHIR99021 at concentration of 10 μM can be added for 24 hours. For the second step, induced cells can be re-seeded into a suitable medium, e.g., composed of RPMI1640 supplemented with B27 supplement with or without vitamin A and the following ligands can be added: for producing Cell type A (may also be referred to as “ground”): CHIR99021 at 5 μM together with FGF2 at 100 ng/ml and TGF pathway inhibitor SB431542 at 10 μM; or for producing Cell type B (may also be referred to as “primed”): CHIR99021 at 5 μM with or without SB431542 at 10 μM; or for producing Cell type C (may also be referred to as “intermediate”): CHIR99021 at 7.5 μM with or without SB431542 at 10 μM; Preferably, these media compositions can be maintained throughout the establishment phase (e.g., 9 passages) and beyond.

In a further preferred embodiment, the methods and axial cells of the present invention have one or more of the following characteristics: the cells after the initial 24-hour induction phase are characterized by the induced expression in all or some cells of markers for early primitive streak, e.g., Brachyury (TBXT), MIXL1, GSC, CDX1, CDX2, EOMES, and/or EVX1. Preferably, at the same time these cells do not express Pax6 (neuroderm) or Sox17 (endoderm). Further preferably, at the same time, pluripotency genes POU5F1, NANOG and SOX2 do not show significant reduction in expression. Further most preferably, the multipotent axial stem cells of the present invention at passage 9 and beyond have one or more of the following characteristics: indefinite self-renewal capability; capable of growing in small to medium tightly formed colonies with epithelial-like morphology; non-teratogenic (i.e., do not form teratomas).

In a further preferred embodiment, axial stem cells of the present invention of type A: co-express TBXT (Brachyury), and SOX2; and preferably also express MIXL1, further preferably express CDX2; further most preferably may express most of the HOX cluster genes (e.g., from A1 to D13); further further most preferably do not express Pax6 or POU5F1; wherein NANOG expression may be heavily downregulated.

In a further preferred embodiment, axial stem cells of the present invention of type B: express SOX2 and most of the HOX cluster genes (e.g., A1 to D9/A10), preferably express Pax6 at high level.; further preferably do not express TBXT (Brachyury), MIXL1 and CDX2.

In a further preferred embodiment, axial stem cells of the present invention of type C: express most of the HOX cluster genes (e.g., A1 to D9/A10), preferably co-express Brachyury (TBXT) and SOX2; further preferably express Pax6 at medium level.

In a further preferred embodiment, all three axial cell types of the present invention (A, B and C) can be further differentiated into spinal motor neurons, osteocytes and chondrocytes. The neuronal differentiation is uniform and required significantly less time compared to established protocols starting from pluripotent cells (e.g., conversion can be observed at 48-72 hours).

In further preferred embodiments, the axial stem cells of the present invention are not neuro-mesodermal progenitors (NMps).

In further preferred embodiments, the methods and uses of the present invention do not produce neuro-mesodermal progenitors (NMps).

In further preferred embodiments, the axial stem cells, composition, preparation or kit of the present invention are used in a method of the present invention (e.g., said method is in vivo, in vitro or ex vivo method).

In further preferred embodiments the axial stem cell, composition, preparation or kit or method of the present invention does not comprise destruction of an embryo (e.g., a human embryo), e.g., is obtainable without destruction of an embryo (e.g., a human embryo).

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A method for producing (or derivation of) axial stem cells (AxSCs) (or neuromuscular-skeletal stem cells), said method comprising:
   a) providing:
      pluripotent stem cells, embryonic or induced pluripotent stem cells (e.g., human PSCs and/or ESC and/or iPSCs, e.g., human ESC line H9 (WA09) or human iPSC line HMGU #1), preferably prior to said derivation/producing said pluripotent cells are maintained in a suitable pluripotent cell media (e.g., mTESR1 or iPS-Brew etc.), further preferably said suitable pluripotent cell media is replaced with RPM11640 medium supplemented with B27 supplement with or without vitamin A for said derivation/producing (i.e. prior to said derivation/producing);
   b) activating the Wnt/β-catenin signaling pathway (e.g., GO:0016055; e.g., Wnt/β-catenin signaling pathway is the series of molecular signals initiated by binding of a Wnt protein to a frizzled family receptor on the surface of the target cell and ending with a change in cell state (Huelsken J, Birchmeier W. New aspects of Wnt signalling pathways in higher vertebrates. Current Opinion in Genetics & Development. 2001 October; 11(5):547-553.); in said pluripotent stem cells, embryonic or induced pluripotent stem cells, preferably said activating is carried out by the means of using an inhibitor of GSK3b protein (e.g., UniProtKB-P49841 or SEQ ID NO: 28), further preferably said inhibitor is CHIR99021, further most preferably said CHIR99021 inhibitor is used at a concentration from about 5 μM to about 10 μM; further most preferably said CHIR99021 inhibitor is used for about 24 hours;
   c) passaging the cells derived from the step (b) under the condition of continuous activation the Wnt/β-catenin signaling pathway in said cells during said passaging, preferably said passaging is carried out for at least about 3 to 9 times (e.g., 3, 4, 5, 6, 7, 8, or 9), further preferably said passaging is a serial passaging, further most preferably said passaging comprises re-seeding (e.g., re-seeding is the dissociation of colonies to single cells or clumps of cells and their plating) the cells derived from the step (b) at a lower density into a fresh serum-free medium (e.g., RPMI1640 medium supplemented with B27 supplement with or without vitamin A); preferably said continuous activation of the Wnt/β-catenin signaling pathway is carried out by the means of using an inhibitor of the Wnt/β-catenin signaling pathway, further preferably said inhibitor is CHIR99021, further most preferably said CHIR99021 inhibitor is used at a concentration of at least about 5 μM (e.g., about 7.5 μM); wherein the cells derived from the step (c) are endogenously expressing transcription factor SOX-2 (e.g., UniProtKB-P48431 or SEQ ID NO: 21);
   d) optionally, said continuous activating the Wnt/β-catenin signaling pathway from the step (c) is carried out in the presence of: Fibroblast growth factor 2 (e.g., UniProtKB-P09038 or SEQ ID NO: 29); and/or a TGF-β (e.g., UniProtKB-P01137 or SEQ ID NO: 30) inhibitor; wherein preferably:
      (d1) said continuous activating the Wnt/β-catenin signaling from the step (c) is carried out with CHIR99021 inhibitor at a concentration of about 5 μM in the presence of: Fibroblast growth factor 2 (e.g., UniProtKB-P09038 or SEQ ID NO: 29) and a TGF-β (e.g., UniProtKB-P01137 or SEQ ID NO: 30) inhibitor; further preferably said TGF-β inhibitor is SB-431542, further most preferably said SB-431542 inhibitor is used at a concentration of about 10 μM, further further most preferably said Fibroblast growth factor 2 is used at a concentration from about 20 to about 100 ng/ml; wherein the cells derived from the step (d1) are: endogenously expressing: transcription factor SOX-2 (e.g., UniProtKB-P48431 or SEQ ID NO: 21), T-box transcription factor T (e.g., UniProtKB-O15178 or SEQ ID NO: 24) and Homeobox protein MIXL1 (e.g., UniProtKB-Q9H2W2 or SEQ ID NO: 26); not endogenously expressing: paired box protein Pax-6 (e.g., UniProtKB-P26367 or SEQ ID NO: 27); optionally, are further endogenously expressing: Homeobox protein CDX-2 (e.g., UniProtKB-Q99626 or SEQ ID NO: 25); or
      (d2) said continuous activating the Wnt/β-catenin signaling from the step (c) is carried out with CHIR99021 inhibitor at a concentration of about 5 μM, preferably in the presence of a TGF-β (e.g., UniProtKB-P01137 or SEQ ID NO: 30) inhibitor, further preferably said TGF-β inhibitor is SB-431542, further most preferably said SB-431542 inhibitor is used at a concentration of about 10 μM; wherein the cells derived from the step (d2) are: endogenously expressing: transcription factor SOX-2 (e.g., UniProtKB-P48431 or SEQ ID NO: 21) and paired box protein Pax-6 (e.g., UniProtKB-P26367 or SEQ ID NO: 27); not endogenously expressing: T-box transcription factor T (e.g., UniProtKB-O15178 or SEQ ID NO: 24), Homeobox protein MIXL1 (e.g., UniProtKB-Q9H2W2 or SEQ ID NO: 26) and Homeobox protein CDX-2 (e.g., UniProtKB-Q99626 or SEQ ID NO: 25); or
      (d3) said continuous activating the Wnt/β-catenin signaling from the step (c) is carried out with CHIR99021 inhibitor at a concentration of about 7.5 μM, preferably in the presence of a TGF-β (e.g., UniProtKB-P01137 or SEQ ID NO: 30) inhibitor, further preferably said TGF-β inhibitor is SB-431542, further most preferably said SB-431542 inhibitor is used at a concentration of about 10 μM, wherein the cells derived from the step (d3) are: endogenously expressing transcription factor SOX-2 (e.g., UniProtKB-P48431 or SEQ ID NO: 21), T-box transcription factor T (e.g., UniProtKB-O15178 or SEQ ID NO: 24), Homeobox protein MIXL1 (e.g., UniProtKB-Q9H2W2 or SEQ ID NO: 26) and paired box protein Pax-6 (e.g., UniProtKB-P26367 or SEQ ID NO: 27); optionally, are further endogenously expressing Homeobox protein CDX-2 (e.g., UniProtKB-Q99626 or SEQ ID NO: 25).
2. The method for producing (or derivation of) axial stem cells (or neuromuscular-skeletal stem cells) according to item 1, comprising step (d).
3. The method for producing (or derivation of) axial stem cells (or neuromuscular-skeletal stem cells) according to any one of the preceding items, comprising step (d); and further comprising: step (d1), (d2) or (d3).
4. The method for producing (or derivation of) axial stem cells (or neuromuscular-skeletal stem cells) according to any one of the preceding items, further comprising passaging the cells derived from the step (c) and/or step (d).
5. The method for producing (or derivation of) axial stem cells (or neuromuscular-skeletal stem cells) according to any one of the preceding items, wherein said axial stem cells (or neuromuscular-skeletal stem cells) have one or more of the following characteristics:
   i) expressing transcription factor SOX-2 (e.g., UniProtKB-P48431 or SEQ ID NO: 21);
   ii) substantially not expressing OCT4 transcription factor (e.g., UniProtKB-Q01860 or SEQ ID NO: 22);
   iii) substantially not expressing homeobox protein NANOG (e.g., UniProtKB-Q9H9S0 or SEQ ID NO: 23);
   iv) are not pluripotent;
   v) are a region-specific multipotent stem cells;
   vi) are obtainable from a pluripotent stem cell, embryonic or induced pluripotent stem cell (e.g., human PSCs and/or ESC and/or iPSCs, e.g., human ESC line H9 (WA09) or human iPSC line HMGU #1);
   vii) are not capable of differentiating into cell types of all tissues of the embryo;
   viii) are only capable of differentiating in cell types emerging from the region of the central body axis during embryonic development (e.g., sclerotome, dermomyotome and peripheral neurons);
   ix) are not capable to form teratomas;
   x) are capable of mimicking the characteristics of the precursors that give rise to the axial region (e.g., motor neurons, peripheral neurons, peripheral nervous system neurons sensory neurons, bone, cartilage, tendon, ligament and/or skeletal muscle cells);
   xi) are not transient cells;
   xii) are capable of differentiating into a motor neuron, peripheral neuron, muscle, cartilage or bone progenitor;
   xiii) are indefinitely renewing stem cells;
   xiv) are capable to grow as clones;
   xv) are not neuromesodermal progenitors (NMps) and/or
   xvi) is not induced neural stem cells (iNSCs).
6. The method for producing axial stem cells according to any one of the preceding items, wherein said axial stem cells are not capable of differentiating into cell types of all tissues of the embryo.
7. The method for producing axial stem cells according to any one of the preceding items, wherein said axial stem cells are only capable of differentiating in cell types emerging from the region of the central body axis during embryonic development.
8. The method for producing axial stem cells according to any one of the preceding items, wherein said axial stem cells are not capable to form teratomas.
9. The method for producing axial stem cells according to any one of the preceding items, wherein said axial stem cells are not transient cells.
10. The method for producing axial stem cells according to any one of the preceding items, wherein said axial stem cells are indefinitely renewing stem cells.
11. The method for producing axial stem cells according to any one of the preceding items, wherein said axial stem cells are capable to grow as clones.
12. The method for producing (or derivation of) axial stem cells (or neuromuscular-skeletal stem cells) according to any one of the preceding items, wherein said axial stem cells (or neuromuscular-skeletal stem cells) are not neuro-mesodermal progenitors (NMps).
13. The method for producing axial stem cells according to any one of the preceding items, wherein said axial stem cells are not neuromesodermal progenitors (NMps), wherein said axial stem cells expressing one or more of the following proteins:
   i) N-myc proto-oncogene protein (MYCN), preferably having UniProtKB-P04198 or SEQ ID NO: 32;
   ii) Protein lin-28 homolog B (LIN28B), preferably having UniProtKB-Q6ZN17 or SEQ ID NO: 31;
   iii) Iroquois-class homeodomain protein IRX-3 (IRX3), preferably having UniProtKB-P78415 or SEQ ID NO: 34;
   iv) Transcription factor SOX-1 (SOX1), preferably having UniProtKB-O00570 or SEQ ID NO: 35;
   v) Zinc finger protein ZIC 2 (ZIC2), preferably having UniProtKB-O95409 or SEQ ID NO: 33;
   vi) Transcription factor SOX-11 (SOX11), preferably having UniProtKB-P35716 or SEQ ID NO: 36;
   vii) preferably, said AxSCs are ground state AxSCs expressing MYCN, LIN28B, ZIC2 and SOX11 as defined in (i)-(vi);
   viii) preferably, said AxSCs are primed state AxSCs expressing MYCN, LIN28B, IRX3, SOX1, ZIC2 and SOX11 as defined in (i)-(vi).
14. The method for producing (or derivation of) axial stem cells (or neuromuscular-skeletal stem cells) according to any one of the preceding items, wherein said axial stem cells (or neuromuscular-skeletal stem cells) are capable of indefinitely renewing and differentiating into:
   i) ground axial stem cells (e.g., indefinitely renewing ground axial stem cells, also referred herein as “CFS”), wherein said ground axial stem cell, expressing:
      a) transcription factor SOX-2 (e.g., UniProtKB-P48431 or SEQ ID NO: 21);
      b) T-box transcription factor T (e.g., UniProtKB-O15178 or SEQ ID NO: 24);
      c) homeobox protein CDX-2 (e.g., UniProtKB-Q99626 or SEQ ID NO: 25); and
      d) homeobox protein MIXL1 (e.g., UniProtKB-Q9H2W2 or SEQ ID NO: 26);
      e) preferably further expressing, homeobox protein Nkx-2.1 (NKX2.1), e.g., UniProtKB-P43699 or SEQ ID NO: 37;
      f) preferably further expressing, protein lin-28 homolog B (LIN28B), e.g., having UniProtKB-Q6ZN17 or SEQ ID NO: 31;
      g) preferably further expressing, N-myc proto-oncogene protein (MYCN), e.g., having UniProtKB-P04198 or SEQ ID NO: 32;
      h) preferably further expressing, E3 ubiquitin-protein ligase TRIM71 (TRIM71), e.g., having UniProtKB-Q2Q1W2 or SEQ ID NO: 38;
      i) preferably further expressing, forkhead box protein B1 (FOXB1), e.g., having UniProtKB-Q99853 or SEQ ID NO: 39;
   ii) primed state axial stem cells (e.g., indefinitely renewing primed state axial stem cells, also referred herein as “CS”), wherein said primed axial stem cell, expressing:
      j) transcription factor SOX-2 (e.g., UniProtKB-P48431 or SEQ ID NO: 21) and
      k) paired box protein PAX-6 (e.g., UniProtKB-P26367 or SEQ ID NO: 27);
      l) preferably further expressing, protein lin-28 homolog B (LIN28B), e.g., having UniProtKB-Q6ZN17 or SEQ ID NO: 31;
      m) preferably further expressing, N-myc proto-oncogene protein (MYCN), e.g., having UniProtKB-P04198 or SEQ ID NO: 32;
      n) preferably further expressing, E3 ubiquitin-protein ligase TRIM71 (TRIM71), e.g., having UniProtKB-Q2Q1W2 or SEQ ID NO: 38;
      o) preferably further expressing, forkhead box protein B1 (FOXB1), e.g., having UniProtKB-Q99853 or SEQ ID NO: 39.
15. The method for producing (or derivation of) an axial stem cells or neuromuscular-skeletal stem cells) according to any one of the preceding items, wherein said axial stem cells or neuromuscular-skeletal stem cells) are human axial stem cells (or human neuromuscular-skeletal stem cells).
16. An axial stem cell (or neuromuscular-skeletal stem cell) produced by the method for producing (or derivation of) axial stem cells (or neuromuscular-skeletal stem cells) according to any one of the preceding items.
17. An isolated axial stem cell (AxSC) (or neuromuscular-skeletal stem cell), wherein said axial stem cell (or neuromuscular-skeletal stem cell) expressing the transcription factor SOX-2 (e.g., UniProtKB-P48431 or SEQ ID NO: 21), substantially not expressing OCT4 transcription factor (e.g., UniProtKB-Q01860 or SEQ ID NO: 22) and substantially not expressing homeobox protein NANOG (e.g., UniProtKB-Q9H9S0 or SEQ ID NO: 23), wherein said AxSC (or neuromuscular-skeletal stem cell) is not pluripotent, wherein said AxSC (or neuromuscular-skeletal stem cell) further has one or more of the following characteristics:
   i) is a region-specific multipotent stem cell;
   ii) is obtainable from a pluripotent stem cell, embryonic or induced pluripotent stem cell (e.g., ESC and/or iPSCs, e.g., human ESC line H9 (WA09) or human iPSC line HMGU #1);
   iii) is not capable of differentiating into cell types of all tissues of the embryo;
   iv) is only capable of differentiating in cell types emerging from the region of the central body axis during embryonic development (e.g., sclerotome, dermomyotome and peripheral neurons);
   v) is not capable to form teratomas;
   vi) is capable of mimicking the characteristics of the precursors that give rise to the axial region (e.g., motor neurons, peripheral neurons, peripheral nervous system neurons sensory neurons, bone, cartilage, tendon, ligament and/or skeletal muscle cells);
   vii) is not a transient cell;
   viii) is capable of differentiating into a motor neuron, peripheral neuron, muscle, cartilage or bone progenitor;
   ix) is an indefinitely renewing stem cell;
   x) is capable to grow as clones;
   xi) is not a neuromesodermal progenitor (NMp);
   xii) is not an induced neural stem cell (iNSC).
18. The axial stem cell (or neuromuscular-skeletal stem cell) according to any one of the preceding items, wherein said axial stem cell (or neuromuscular-skeletal stem cell) is not capable of differentiating into cell types of all tissues of the embryo.
19. The axial stem cell (or neuromuscular-skeletal stem cell) according to any one of the preceding items, wherein said axial stem cell (or neuromuscular-skeletal stem cell) is only capable of differentiating in cell types emerging from the region of the central body axis during embryonic development.
20. The axial stem cell (or neuromuscular-skeletal stem cell) according to any one of the preceding items, wherein said axial stem cell (or neuromuscular-skeletal stem cell) is not capable to form teratomas.
21. The axial stem cell (or neuromuscular-skeletal stem cell) according to any one of the preceding items, wherein said axial stem cell (or neuromuscular-skeletal stem cell) is not a transient cell.
22. The axial stem cell (or neuromuscular-skeletal stem cell) according to any one of the preceding items, wherein said axial stem cell (or neuromuscular-skeletal stem cell) is an indefinitely renewing stem cell.
23. The axial stem cell (or neuromuscular-skeletal stem cell) according to any one of the preceding items, wherein said axial stem cell (or neuromuscular-skeletal stem cell) is capable to grow as clones.
24. The axial stem cell (or neuromuscular-skeletal stem cell) according to any one of the preceding items, wherein said axial stem cell (or neuromuscular-skeletal stem cell) is not a neuromesodermal progenitor (NMp).
25. The axial stem cell (or neuromuscular-skeletal stem cell) according to any one of the preceding items, wherein said axial stem cell (or neuromuscular-skeletal stem cell) is not a neuromesodermal progenitor (NMp), wherein said axial stem cells expressing one or more of the following proteins:
   i) N-myc proto-oncogene protein (MYCN), preferably having UniProtKB-P04198 or SEQ ID NO: 32;
   ii) Protein lin-28 homolog B (LIN28B), preferably having UniProtKB-Q6ZN17 or SEQ ID NO: 31;
   iii) Iroquois-class homeodomain protein IRX-3 (IRX3), preferably having UniProtKB-P78415 or SEQ ID NO: 34;
   iv) Transcription factor SOX-1 (SOX1), preferably having UniProtKB-O00570 or SEQ ID NO: 35;
   v) Zinc finger protein ZIC 2 (ZIC2), preferably having UniProtKB-O95409 or SEQ ID NO: 33;
   vi) Transcription factor SOX-11 (SOX11), preferably having UniProtKB-P35716 or SEQ ID NO: 36;
   vii) preferably, said AxSC is a ground state AxSC expressing MYCN, LIN28B, ZIC2 and SOX11 as defined in (i)-(vi);
   viii) preferably, said AxSC is a primed state AxSC expressing MYCN, LIN28B, IRX3, SOX1, ZIC2 and SOX11 as defined in (i)-(vi).
26. The axial stem cell (or neuromuscular-skeletal stem cell) according to any one of the preceding items, wherein said axial stem cell (or neuromuscular-skeletal stem cell) is capable of indefinitely renewing itself and differentiating into:
   i) a ground axial stem cell (e.g., indefinitely renewing ground axial stem cell, also referred herein as “CFS”), wherein said ground axial stem cell, expressing:
      a) transcription factor SOX-2 (e.g., UniProtKB-P48431 or SEQ ID NO: 21);
      b) T-box transcription factor T (e.g., UniProtKB-O15178 or SEQ ID NO: 24);
      c) homeobox protein CDX-2 (e.g., UniProtKB-Q99626 or SEQ ID NO: 25); and
      d) homeobox protein MIXL1 (e.g., UniProtKB-Q9H2W2 or SEQ ID NO: 26);
      e) preferably further expressing, homeobox protein Nkx-2.1 (NKX2.1), e.g., UniProtKB-P43699 or SEQ ID NO: 37;
      f) preferably further expressing, protein lin-28 homolog B (LIN28B), e.g., having UniProtKB-Q6ZN17 or SEQ ID NO: 31;
      g) preferably further expressing, N-myc proto-oncogene protein (MYCN), e.g., having UniProtKB-P04198 or SEQ ID NO: 32;
      h) preferably further expressing, E3 ubiquitin-protein ligase TRIM71 (TRIM71), e.g., having UniProtKB-Q2Q1W2 or SEQ ID NO: 38;
      i) preferably further expressing, forkhead box protein B1 (FOXB1), e.g., having UniProtKB-Q99853 or SEQ ID NO: 39;
   ii) primed state axial stem cell (e.g., indefinitely renewing primed state axial stem cell, also referred herein as “CS”), wherein said primed axial stem cell, expressing:
      j) transcription factor SOX-2 (e.g., UniProtKB-P48431 or SEQ ID NO: 21) and
      k) paired box protein PAX-6 (e.g., UniProtKB-P26367 or SEQ ID NO: 27);
      l) preferably further expressing, protein lin-28 homolog B (LIN28B), e.g., having UniProtKB-Q6ZN17 or SEQ ID NO: 31;
      m) preferably further expressing, N-myc proto-oncogene protein (MYCN), e.g., having UniProtKB-P04198 or SEQ ID NO: 32;
      n) preferably further expressing, E3 ubiquitin-protein ligase TRIM71 (TRIM71), e.g., having UniProtKB-Q2Q1W2 or SEQ ID NO: 38;
      o) preferably further expressing, forkhead box protein B1 (FOXB1), e.g., having UniProtKB-Q99853 or SEQ ID NO: 39.
27. The axial stem cell (or neuromuscular-skeletal stem cell) according to any one of the preceding items, wherein said axial stem cell (or neuromuscular-skeletal stem cell) is a human axial stem cell (or human neuromuscular-skeletal stem cell).
28. The method for producing (or derivation of) axial stem cells (or neuromuscular-skeletal stem cells) according to any one of the preceding items, wherein said axial stem cells (or neuromuscular-skeletal stem cells) are the axial stem cells (or neuromuscular-skeletal stem cells) according to any one of the preceding items.
29. A composition, preparation or kit comprising the axial stem cell (or neuromuscular-skeletal stem cell) according to any one of the preceding items.
30. The composition, preparation or kit according to any one of preceding items, wherein said composition, preparation or kit is a pharmaceutical and/or diagnostic composition, preparation or kit.
31. The axial stem cell, composition, preparation or kit according to any one of the preceding items for use as a medicament.
32. The axial stem cell (or neuromuscular-skeletal stem cell), composition, preparation or kit according to any one of the preceding items for use in one or more of the following:
   i) in a method of treatment, amelioration, prophylaxis and/or diagnostics of a neurodegenerative disease;
   ii) in a method of treatment, amelioration, prophylaxis and/or diagnostics of a bone and/or cartilage disorder;
   iii) in a method of treatment, amelioration, prophylaxis and/or diagnostics of muscle disorder;
   iv) in a method of regenerative treatment of a cell, tissue, organ and/or body;
   v) in a method for screening a candidate compound for activity against a disease (e.g., a disease of the peripheral nervous system or a disease relating to axial stem cells, e.g. muscle-, motor neuron-, peripheral neuron-, sensory neuron cartilage-, tendon-related (e.g., degenerative) disease) and/or for neurotoxicity screening;
   vi) in a method for treatment, amelioration, prophylaxis and/or diagnostics of a disease relating to axial stem cells, e.g. muscle-, motor neuron-, peripheral neuron-, sensory neuron cartilage-, tendon-related (e.g., degenerative) disease;
   vii) in any of (i)-(vi), wherein said method is an in vitro, ex vivo or in vivo method.
33. A method for improving the condition of a sample or subject in need thereof, wherein said method is one or more of the following:
   i) a method of treatment, amelioration, prophylaxis and/or diagnostics of a neurodegenerative disease;
   ii) a method of treatment, amelioration, prophylaxis and/or diagnostics of a bone and/or cartilage disorder;
   iii) a method of treatment, amelioration, prophylaxis and/or diagnostics of muscle disorder;
   iv) a method of regenerative treatment of a cell, tissue, organ and/or body;
   v) a method for screening a candidate compound for activity against a disease (e.g., a disease of the peripheral nervous system) and/or for neurotoxicity screening;
   said method comprising:
      a) providing the axial stem cell, composition, preparation or kit according to any one of preceding items; to said sample or subject;
      b) administering a therapeutically effective amount of said the axial stem cell, composition, preparation or kit; to said sample or subject.
34. The method according to any one of the preceding items, wherein said method is an in vitro, ex vivo or in vivo method.
35. Use of the axial stem cell, composition, preparation and/or kit according to any one of the preceding items, for one or more of the following:
   i) for treatment, amelioration, prophylaxis and/or diagnostics of a neurodegenerative disease;
   ii) for treatment, amelioration, prophylaxis and/or diagnostics of a bone and/or cartilage disorder;
   iii) for treatment, amelioration, prophylaxis and/or diagnostics of muscle disorder;
   iv) for regenerative treatment of a cell, tissue, organ and/or body;
   v) for screening a candidate compound for activity against a disease (e.g., a disease of the peripheral nervous system) and/or for neurotoxicity screening;
   vi) in any of (i)-(v), wherein said use is an in vitro, ex vivo or in vivo use.

The invention is further illustrated by the following examples, however, without being limited to the example or by any specific embodiment of the examples.

### EXAMPLES OF THE INVENTION

Material and Methods

Cell Culture

The human ESC line H9 (WA09) and human iPSC line HMGU #1 (Kunze et al., 2018) were grown in either mTesR1 (Stem Cell Technologies) or iPS Brew XP (Miltenyi Biotech) media on Matrigel-coated (BD Corning) plates. Cells were split using Passaging solution XF (Miltenyi Biotech) at 1:10 ratio when confluence >70%. H9 cells were used at passages 42-65, and HMGU #1 passages 21-34. In all experiments fresh medium was applied daily, and cells were cultured with 5% CO2.

In time course experiments cells were dissociated using Accutase (Sigma) and seeded into 12-well plates at 2.5×105 cells per well in mTesR1 medium supplemented with 10 μM Y-27632 (R&D). 24 hours later medium was replaced with differentiation medium: RPMI-1640 with L-Glutamine and 1× B-27 supplement without insulin containing 10 μM CHIR99021 (Tocris). In case of beta-catenin overexpression, the procedure was the same, except that CHIR99021 was replaced with 1 μg/ml doxycycline (Clontech). All cell culture media components were from Life Technologies unless indicated otherwise.

Axial Stem Cell Derivation

Cells were seeded as described above and treated with 10 μM CHIR for 24 hours. Afterwards cells were split at 1:20-1:30 ratio into Matrigel-coated 6-well plates containing maintenance medium (RPMI-1640 supplemented with L-Glutamine, 1× non-essential amino acids, 1× B-27 supplement without vitamin A and the respective ligands (see Table 1) and the cultures were incubated until they became confluent. Confluent cultures were routinely split at 1:10-1:20 ratio until the end of establishment phase defined at passage 9. Splitting was done with either Passaging solution XF or Versene (Life Technologies). FGF2-containing medium was prepared freshly every week, the media without FGF2—at least every 2 weeks. No manual colony picking or scraping off the differentiated cells was employed at any stage during the establishment of cell lines.

TABLE 1
Ligands

| Ligand | Manufacturer | Stock: | | Used at: | |
|---|---|---|---|---|---|
| CHIR99021 | Tocris | 10 | mM in water | 1, 3, 5, | |
| trihydrochloride | Bioscience | | | 7.5, 10 μM | |
| Human | Peprotech | 200 | μg/ml in PBS | 100 | ng/ml |
| recombinant | | | | | |
| FGF2 | | | | | |
| SB431542 | Miltenyi | 10 | mM in DMSO | 10 | μM |
| | Biotech | | | | |
| Human | Peprotech | 10 μg/ml in 4 mM | | 10 | ng/ml |
| recombinant | | HCl + 0.1% BSA | | | |
| TGF-β1 | | | | | |
| Dorsomorphin | Tocris | 1 | mM in DMSO | 0.1 | μM |
| dihydrochloride | Bioscience | | | | |

| | | | | |
|---|---|---|---|---|
| Human | R&D Systems | 25 μg/ml in 4 mM | 10 | g/ml |
| recombinant | | HCl + 0.1% BSA | | |
| BMP4 | | | | |

ΔN90 β-Catenin hESC Line

To generate tetracycline-inducible overexpression of constitutively active β-catenin (contains deletion of the first 90 aa from the N-terminus), H9 cells were nucleofected with PB-GFP-P2A-ΔNβCAT plasmid and a Piggybac transposase-coding plasmid using P3 primary cell 4D nucleofector kit (Lonza). 48 hours later, cells were split into 10-cm dish and selected using 50 μg/ml Hygromycin B (Life Technologies) for 2 weeks. Afterwards, polyclonal stable line was propagated in the same way as parental H9 line in the presence of 25 μg/ml Hygromycin B.

Directed Differentiation

Motor neuron differentiation: AxSC lines were dissociated with Accutase and 1.5×105 cells seeded into one well of a 12-well plate coated with Matrigel containing respective maintenance medium supplemented with 10 μM Y-27632. Next day, medium was change to neuronal differentiation medium: DMEM/F12 and Neurobasal medium A at 1:1 ratio with 1× B-27 supplement, 1× N2 supplement, 0.1 μM retinoic acid (Sigma), 100 ng/ml recombinant sonic hedgehog (R&D), 10 ng/ml BDNF (R&D), 10 ng/ml GDNF (R&D), 10 ng/ml IGF-1 (Peprotech), 0.1 μM compound E (Merck) and 100 μM cAMP (Sigma). For the first 5 days medium was changed daily, afterward every other day.

Osteocyte differentiation: AxSC lines were seeded as above but at higher density—2.5×105 cells per one well of a 12-well plate. Next day, medium was changed to RPMI-1640 with L-Glutamine and 1× B-27 supplement without vitamin A supplemented with 300 nM SAG (Sigma) and 20 ng/ml FGF2 (Peprotech). After 2 days, medium was changed to OsteoDiff StemMACS medium (Miltenyi Biotech) for the rest of the differentiation. Medium was changed daily for the first 5 days, afterwards every other day.

Chondrocyte differentiation: Chondrocytes were differentiated from AxCS lines using same procedure as for osteocytes except that differentiation medium was ChondroDiff StemMACS (Miltenyi Biotech).

Quantitative PCR

Total RNA was isolated using RNeasy mini kit (Qiagen). cDNA was synthesized from 0.2-1 μg of total RNA (normalized amount between samples within each experiment) using Verso cDNA synthesis kit (Thermo Scientific) according to manufacturers instructions. 1 μl of cDNA (1:5 dilutions) were used as template in 10 μl qPCR reactions. PCR was set up using either pre-designed Taqman Gene Expression Assays together with Taqman Gene Expression Master Mix (both from Thermo Scientific) or custom-designed primers together with Power SYBR Green master mix (Thermo Scientific). All primer and Taqman probe details are listed in, e.g., Table 2. Primer concentration in the reactions was 250 nM for all primers. PCR was performed on QuantStudio 12K Flex Real-Time PCR System (Thermo Scientific) using pre-defined cycling parameters. 2 technical replicates were run for each reaction. Taqman assays and primers used are listed in, e.g., Table 2. GAPDH served as a housekeeping gene in all experiments. Relative fold changes (FC) in gene expression were calculated using ΔΔCt method (Livak and Schmittgen, 2001). Results are presented as mean ΔΔCt±standard error of the mean between biological replicates, unless indicated otherwise. Plots were produced using ggplot2 package (Wickham, 2009) in RStudio software running R version 3.5.1 (R Core Team, 2018).

TABLE 2
Genes and qPCR primers and probes.
Primers for SYBR Green qPCR, synthesized by Sigma-Aldrich

| | |
|---|---|
| GAPDH (e.g., UniProtKB - P04406) | F: SEQ ID NO: 1/ |
| | R: SEQ ID NO: 2 |
| MNX1(HB9) (e.g., UniProtKB - P50219) | F: SEQ ID NO: 3/ |
| | R: SEQ ID NO: 4 |
| PRPH (e.g., UniProtKB - P41219) | F: SEQ ID NO: 5/ |
| | R: SEQ ID NO: 6 |
| POU4F1 (e.g., UniProtKB - Q01851) | F: SEQ ID NO: 7/ |
| | R: SEQ ID NO: 8 |
| RUNX2 (e.g, UniProtKB - Q13950) | F: SEQ ID NO: 9/ |
| | R: SEQ ID NO: 10 |
| BGLAP (e.g., UniProtKB - P02818) | F: SEQ ID NO: 11/ |
| | R: SEQ ID NO: 12 |
| COL1A1 (e.g., UniProtKB - P02452) | F: SEQ ID NO: 13/ |
| | R: SEQ ID NO: 14 |
| NKX3-2 (e.g., UniProtKB - P78367) | F: SEQ ID NO: 15/ |
| | R: SEQ ID NO: 16 |
| COMP (e.g., UniProtKB - P49747) | F: SEQ ID NO: 17/ |
| | R: SEQ ID NO: 18 |
| ACAN (e.g., UniProtKB - P16112) | F: SEQ ID NO: 19/ |
| | R: SEQ ID NO: 20 |

Pre-designed Taqman Gene Expression Assays (Thermo Scientific)

| | |
|---|---|
| GAPDH (e.g., UniProtKB - P04406) | Hs02758991_g1 |
| POU5F1 (e.g., UniProtKB - Q01860) | Hs00999632_g1 |
| NANOG (e.g., UniProtKB - Q9H9S0) | Hs04260366_g1 |
| SOX2 (e.g., UniProtKB - P48431) | Hs01053049_S1 |
| T-box transcription factor T (e.g., | Hs00610080_m1 |
| UniProtKB - O15178) | |
| MIXL1 (e.g., UniProtKB - Q9H2W2) | Hs00430824_g1 |
| CDX2 (e.g., UniProtKB - Q99626) | Hs01078080_m1 |
| PAX6 (e.g., UniProtKB - P26367) | Hs00240871_m1 |
| EOMES (e.g., UniProtKB - O95936) | Hs00172872_m1 |
| POU4F1 (e.g., UniProtKB - Q01851) | Hs00366711_m1 |
| ISL1 (e.g., UniProtKB - P61371) | Hs00158126_m1 |
| CHAT (e.g., UniProtKB - P28329) | Hs00758143_m1 |

Immunofluorescent Analysis

For immunofluorescent analysis, cells were seeded onto either matrigel-coated glass coverslips or into matrigel-coated 4-well μ-slides (Ibidi). For IF on differentiated motor neurons, AxCS lines were differentiated directly on the coverslips. Following fixation with 4% methanol-free formaldehyde in PBS (Thermo Scientific) for 15 minutes at room temperature (RT), cells were permeabilized with 0.2% Triton X-100 (Sigma-Aldrich) for 10 minutes and subsequently blocked with 5% goat serum (Sigma-Aldrich) in PBS/0.05% Triton-X100 for 40 minutes at RT. Primary antibody dilutions (e.g., Table 3) in the blocking buffer were added and incubated overnight at 4° C. Next day, cells were washed with PBS 3 times and incubated with secondary antibodies diluted in PBS for 1 hours at RT. Coverslips were mounted using ProLong Gold mounting reagent with DAPI. Images were obtained at 63× magnification using Zeiss Axio Observer.Z1 epifluorescent microscope equipped with Apotome.2 and Zen software (Zeiss).

TABLE 3
Exemplary antibodies and dilutions.

| Antibody | Provider | Used at: |
|---|---|---|
| Rabbit anti-Sox2 | Cell Signaling Technology, | 1:400 |
| | #2748 | |
| Mouse anti-Brachyury, | Thermo Scientific, cat. | 1:100 |
| X1AO2 | 14-9770-82 | |
| Rabbit anti-CDX2 mAb, | Cell Signaling Technology, | 1:200 |
| D11D10 | #12306 | |
| Rabbit anti-Pax6 | Biolegend, cat. 901301 | 1:100 |
| Mouse anti-HB9 mAb | DSHB, cat. 81.5C10 | 1:100 |
| Rabbit anti-Islet 1 | Abcam, cat. ab20670 | 1:200 |
| Rabbit anti- β3-tubulin | Cell Signaling Technology, | 1:200 |
| | #5568 | |
| Goat anti-rabbit IgG (H + L), | Life Technologies, A-11034 | 1:1000 |
| Alexa Fluor 488, highly | | |
| cross-absorbed | | |
| Goat anti-mouse IgG (H + L) | Life Technologies, A32728 | 1:1000 |
| Alexa Fluor Plus 647, highly | | |
| cross-absorbed | | |

Osteocyte and Chondrocyte Staining

Differentiated cells were washed twice with PBS and fixed with 4% formaldehyde for 30 minutes at RT. Following three washed with distilled water, osteocytes were stained with 40 mM alizarin red solution (Sigma-Aldrich) for 30 minutes. Chondrocytes were stained with 1% alcian blue solution in 3% acetic acid, pH 2.5, for 1 hour at RT. Alcian blue was washed once with 3% acetic acid, followed by three washes with distilled water, 15 minutes each wash. Alizarin red wash washed with distilled water, 15 minutes each wash. Images of the wells were acquired using Leica ICC50HD color camera at 10× magnification.

RNA Sequencing

3 μg of total RNA were treated with TURBO DNase (Life Technologies) and purified using RNeasy Minelute RNA cleanup kit (Qiagen). RNA quality was assessed using microcapillary electrophoresis on Agilent 2100 Bioanalyzer with RNA Pico 6000 kit (Agilent) and only RNA with RIN values >8 was processed further. Per RNA-seq library, 1 μg of DNAse-treated RNA was treated with RiboZero Gold (Human/Mouse/Rat) kit (Illumina) to remove rRNAs, followed by RNA cleanup using the RNeasy Minelute RNA cleanup kit. Sequencing libraries were prepared from equal quantities of rRNA-depleted RNA using TruSeq Stranded total RNA LT kit (Illumina) according to manufacturer's instructions using 11 cycles of enrichment PCR. The quality of the libraries was assessed using Agilent 2100 Bioanalyzer with the DNA 1000 kit (Agilent). Library concentration was measured using Qubit dsDNA HS Assay Kit (Life Technologies). Multiplexing of libraries was performed according to manufacturer's instructions. Multiplexed libraries from CHIR time course experiment were sequenced using a NextSeq 500 (Illumina) to generate 75-nt single-end reads. Sequencing depth was 20-40 Mio reads per library. Libraries from established AxCS lines were sequenced on HiSeq2500 machine to generate 50 bp single-end reads. Sequencing depth was 12-14 Mio reads per sample.

RNA-Sequencing Data Analysis

For CHIR timecourse experiment, reads were pseudo-aligned to human transcriptome (Ensembl version GRChg38.86) using kallisto (version 0.43.0_3) (Bray et al., 2016). Resulting estimated transcript-level abundances were aggregated into counts per gene and exported using Tximport (version 1.10.1) pipeline (Soneson et al., 2015). Differential gene expression analysis was performed using DESeq2 package (version 1.22.2) (Love et al., 2014) in R by comparing gene expression at each stimulation time point to undifferentiated parental cell line. Normalized counts were extracted from DESeq2 object. PCA plot was constructed from rlog-transformed raw counts.

For AxCS lines RNA-sequencing, genome alignment and read count steps were performed using Galaxy platform (Afgan et al., 2018). Briefly, reads were trimmed using Trimmomatic (Bolger et al., 2014) with default parameters and aligned to hg38 human genome using HiSAT2 (Kim et al., 2015). Reads were counted from bam files using featureCounts (Liao et al., 2014). Differential gene expression analysis was accomplished using DESeq2 package (version 1.22.2) in R by comparing AxCS lines to undifferentiated parental cell line. Differentially expressed genes from each line were analysed for over-representation of GO ontology terms from “biological process” category using topGO package (Alexa and Rahnenfuhrer, 2018) reporting p-values from Fisher's exact test on normalized gene counts.

Analysis of the over-represented pathways was performed using Genomatix Pathway System (GePS) tool from the Genomatix software suite (http://www.genomatix.de) by providing significantly up-regulated genes from each time point and stimulation as input.

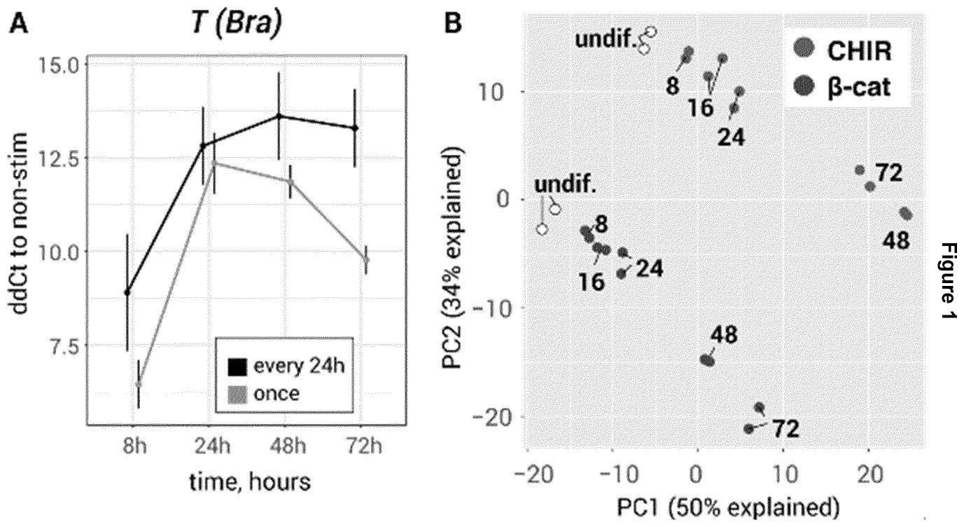

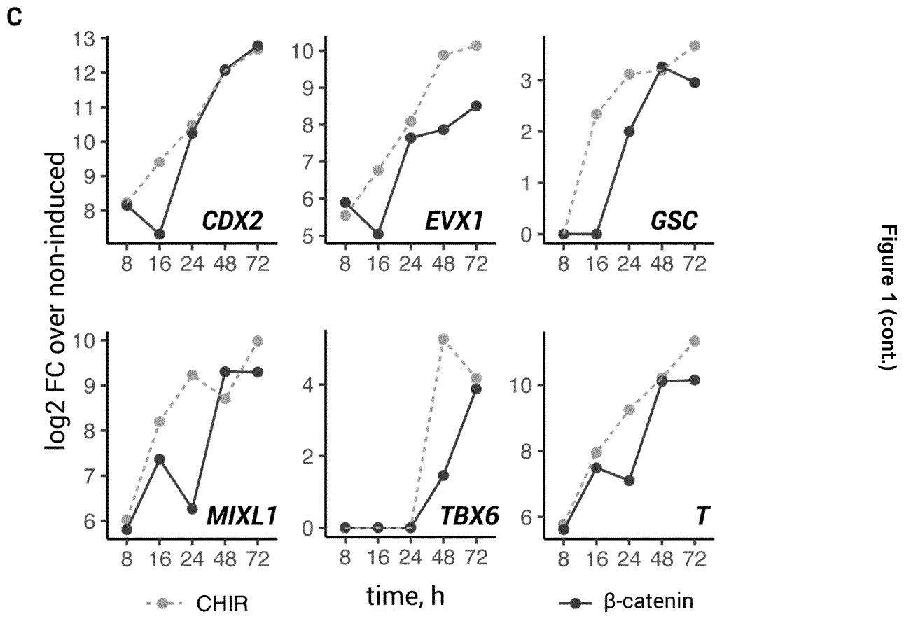

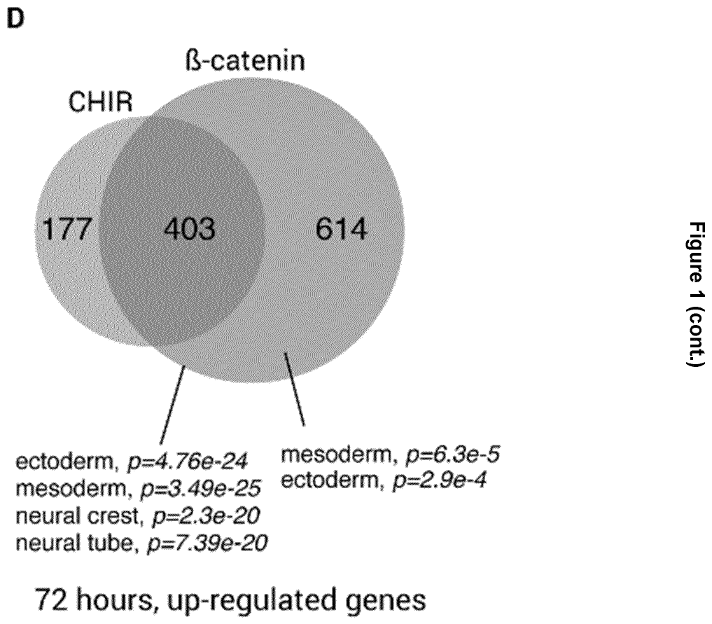

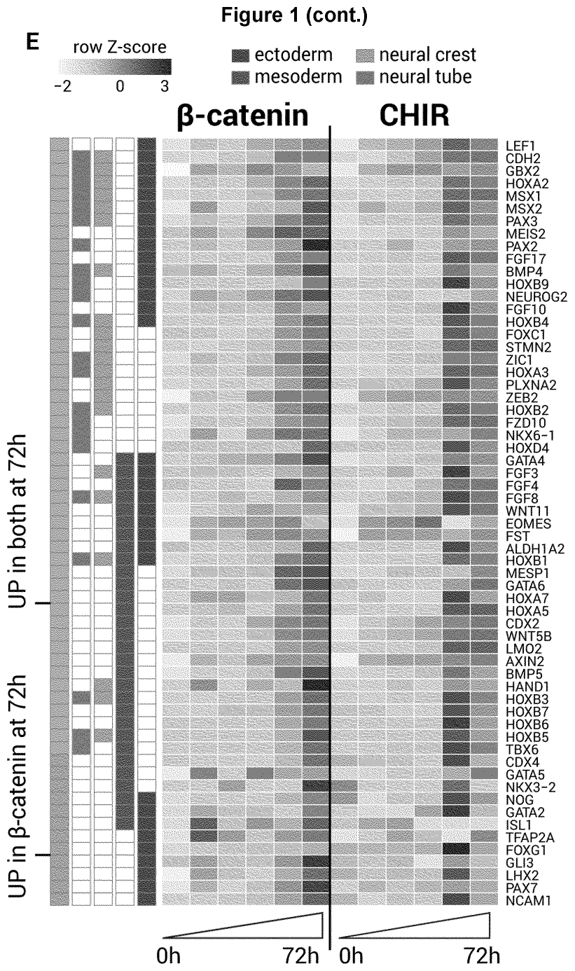

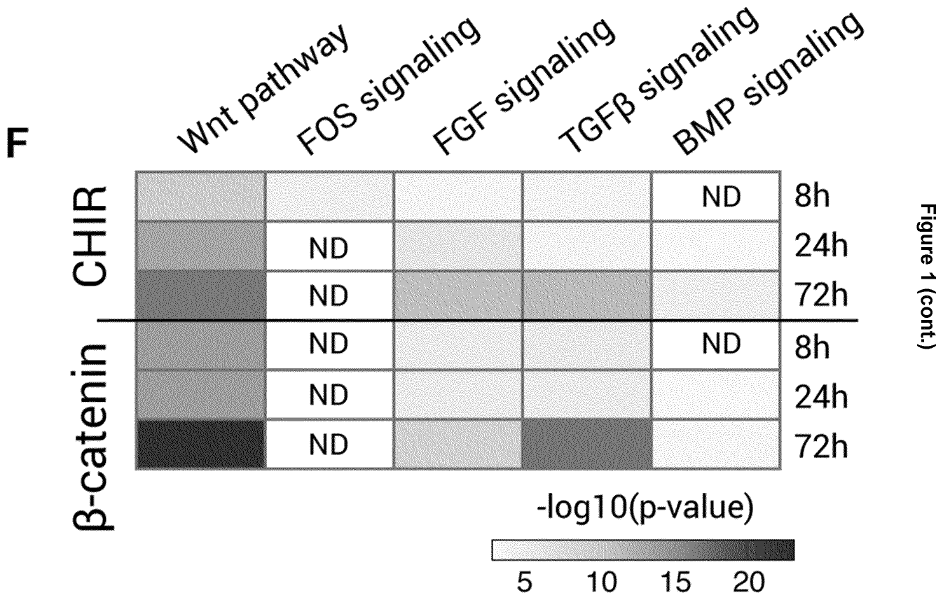

Tissue and cell type associations were analysed using Enrichr tool (Kuleshov et al., 2016) and JENSEN tissue expression database (https://tissues.jensenlab.org). Gene lists associated with selected cell types were extracted and their normalized expression values (from DESeq2 analysis) were used to construct heatmap of the FIG. 1E.

### Example 1: Maintained Expression of TBXT and SOX2 in Passaged Progeny of hESCs

To permanently capture human embryonic axial-like progenitors in vitro we first sought to establish a state resembling their hallmark of SOX2, TBXT/BRA expression. Because SOX2 is already highly expressed in undifferentiated hESCs, we focused on inducing highest possible endogenous TBXT expression by activation of Wnt/β-catenin signaling, which regulates this gene in the early primitive streak (Arnold et al., 2000). We compared continuous activation of Wnt/β-catenin to transient activation for 24 hours, which creates progenitors of lateral mesoderm (Lian et al., 2012) using CHIR99021 as agonist of the pathway. We noted that continuous 10 μM treatment sustained high level of TBXT (e.g., FIG. 1A). To characterize the differentiation stage under continuous WNT activation, we conducted time-series RNA-sequencing (e.g., FIG. 1B). We noted a sharp trend of up-regulation that plateaued within 48-72 hours of genes that are expressed in the primitive streak (TBXT, MIXL1, GSC, EVX1) as well as neural genes (ZIC1, CDH2, GBX2) and genes characteristic of elongating axis (early and immediate members of the HOX gene cluster) (e.g., FIG. 1C-E). Gene ontology analysis confirmed the association of the transcriptome with mesoderm, ectoderm and neural development at the 72 hours time point of continuous CHIR99021 treatment (e.g., FIG. 1D,E). This indicates that axial progenitors and their autonomous regulation begin to form by continuous inhibition of GSK-3β in hESCs.

To confirm that this axial gene signature represents the direct activation of Wnt/β-catenin, we integrated to the genome of hESCs a constitutively active variant of β-catenin under the regulation of tetracycline-controlled transcriptional activation. Continuous treatment of this line with doxycycline revealed similar trends of up-regulation of axis mesodermal and neural genes, albeit constitutively active β-catenin led to induction of an overall broader gene cohort and higher expression, suggesting stronger induction of the pathway (e.g., FIG. 1D,E). Collectively, this indicates that the continuous activation of the Wnt/β-catenin pathway produces progenitors with axial-like identity, which plausibly have developmental potential encompassing paraxial mesodermal lineages and peripheral neural progeny.

Our next goal was to define conditions promoting renewal of axial-like cells in culture upon passaging. To survey the signaling cascades that could be important for renewal and differentiation, we conducted pathway analysis, which revealed, in addition to the externally activated Wnt/β-catenin pathway, endogenous FGF, TGF-β, and to a lesser extent BMP signaling (e.g., FIG. 1F). Because basic FGF (FGF2) is a potent mitogen of neural progenitors (Kitchens et al., 1994) and mesoderm spreading (Wilson et al., 2005), we sought to promote renewal of axial-like progenitors by CHIR99021 treatment with and without FGF2. Even though induction of axial markers did not reach peak levels following 24 hours (e.g., FIG. 1C), we began passaging of the cells at this time point because the level of SOX2 was comparable to the undifferentiated state (e.g., FIG. 1G). Strikingly, we found that a passaging campaign beginning at 24 hours and in presence of CHIR99021 produced progeny stably expressing SOX2 at a comparable level to undifferentiated hESCs, concomitantly with mild and strong down-regulation of NANOG and OCT4 respectively, which are the markers exclusive to the pluripotent state (e.g., FIG. 1H). Surprisingly, although TBXT is a direct target of β-catenin, in these experiments, its sustained expression required the presence of FGF2 (e.g., FIG. 1H). Taken together, these results indicate that permanent activation of Wnt/β-catenin in hESCs and passaging that begins at 24 hours, promote renewal of cells that co-express the axial markers TBXT and SOX2, but neither of the crucial pluripotency-reprogramming factors NANOG and OCT4, that are essential for earlier developmental stages. Strikingly, constant Wnt/β-catenin activation alone sustained a state of cells that maintained SOX2 alone.

### Example 2: Derivation of Stem Cell Lines with Characteristics of Axial States

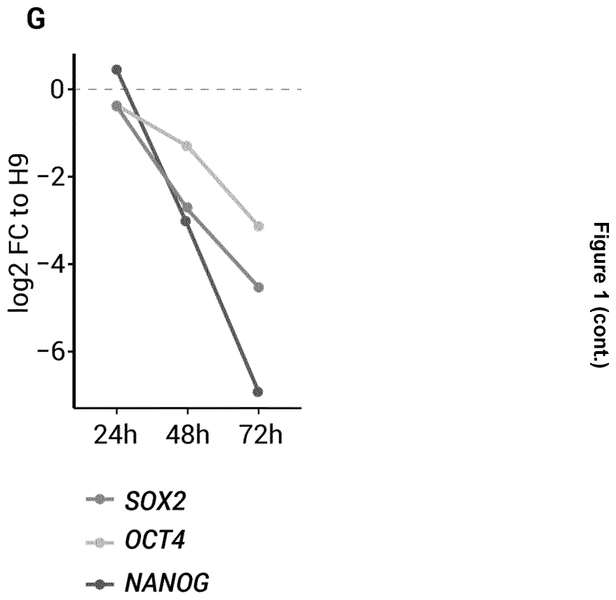

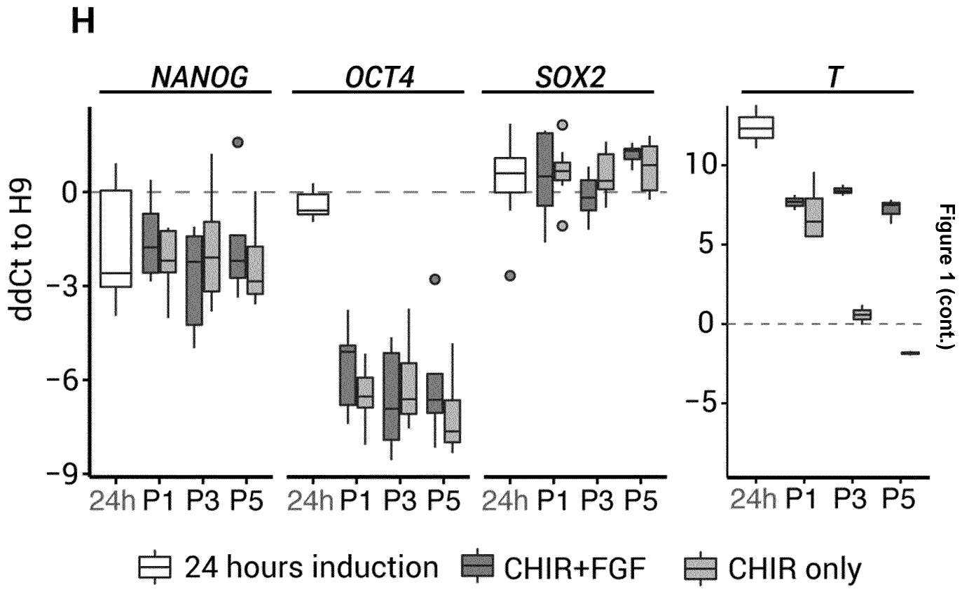

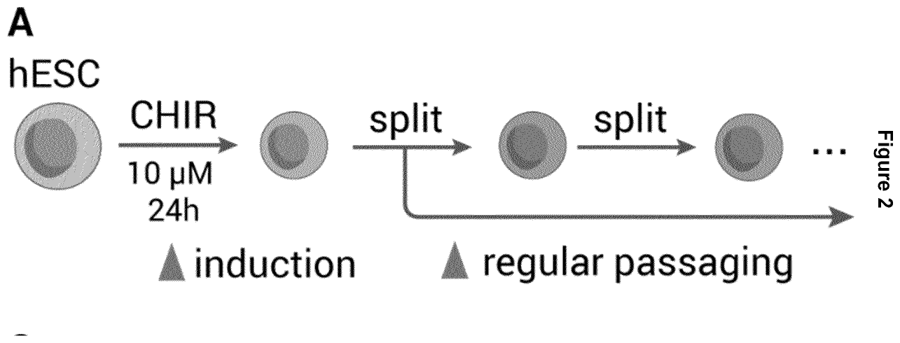

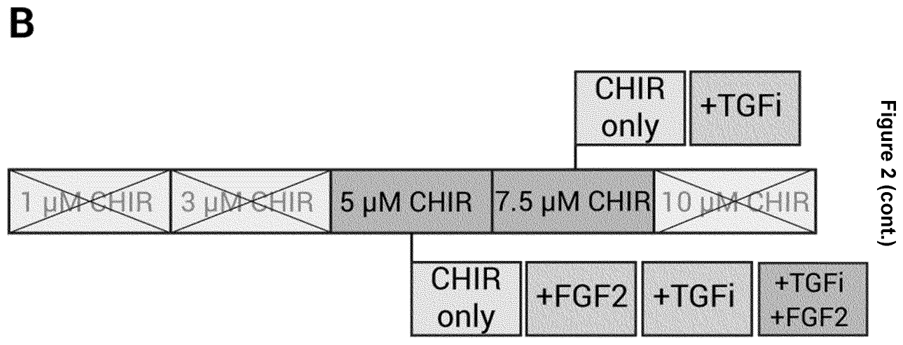

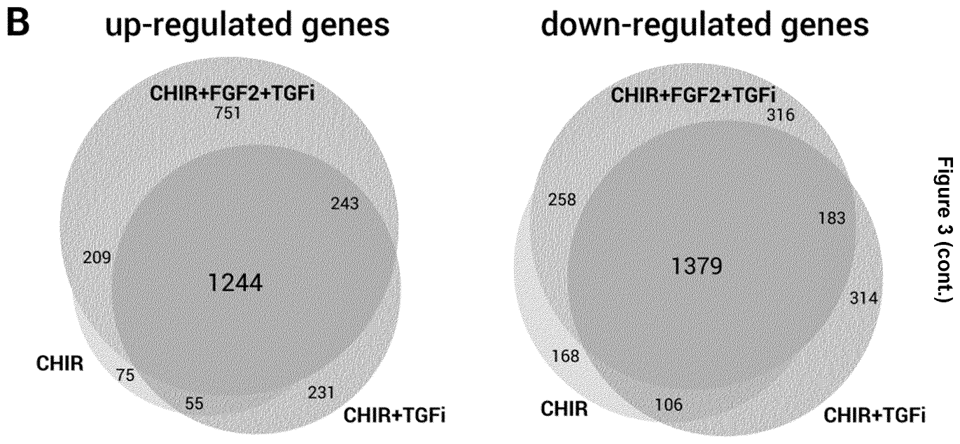

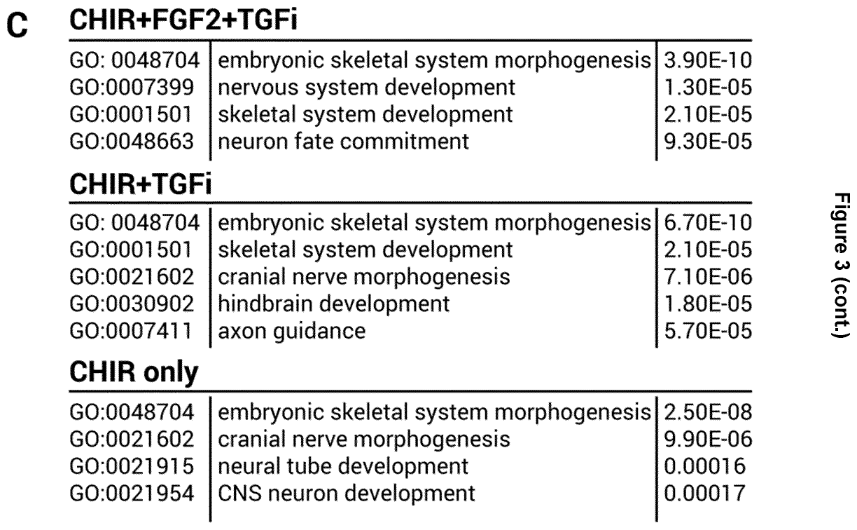

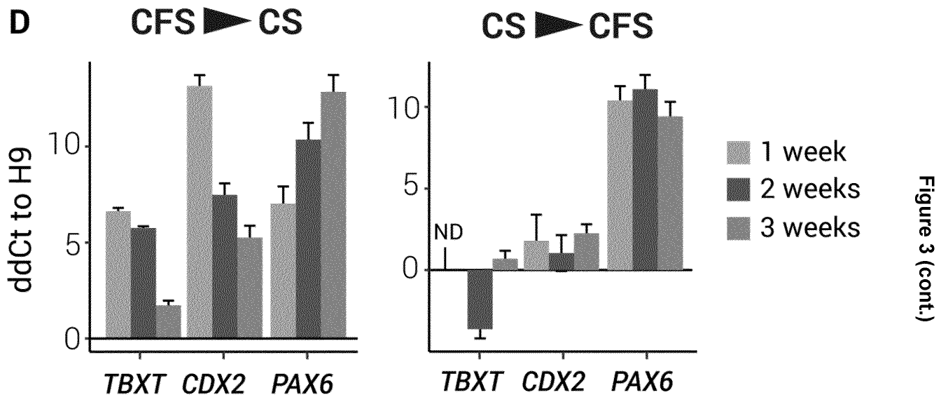

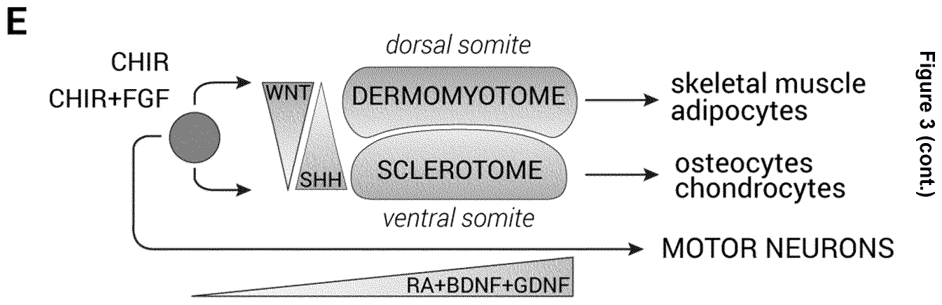

Next, we tested the derivation of stem cell-like lines that represent the two axial states. According to maintenance of SOX2 and TBXT state by Wnt/β-catenin and FGF signaling, or of SOX2 state by Wnt/β-catenin alone, we screened a range of concentrations of CHIR99021, with and without addition of FGF2, and ligands and inhibitors of TGF-β and BMP signaling (e.g., FIG. 4). We examined if applying these conditions after 24 hours of CHIR99021 treatment and serial passaging could establish phenotypically stable lines over months of culture (e.g., FIG. 2A).

The results of three independent serial passaging campaigns using hESCs as the starting population revealed that treatment with 5 or 7.5 μM CHIR99021 enabled the derivation of lines that exhibited compact stem cell colony morphology in the presence or absence of FGF2 (e.g., FIG. 2C,F). Moreover, applying TGF-β inhibitor SB-431542 created more compact cells and denser colonies (e.g., inset FIG. 2C,F). In contrast, addition of BMP4 or TGF-β prohibited the deviation of stable lines altogether (e.g., FIG. 4). Finally, concentration of CHIR99021 lower than 5 μM did not promote the formation of cell lines, and 10μM treatment was toxic to hESC conversion but not to the iPSCs (e.g., FIGS. 2B, 4 and 5). Representative lines that we derived from hESCs were passaged at least 40 times, totaling to 8 months of continuous culture or more without exhibiting overt changes in proliferation rate, viability, or epithelial morphology (e.g., FIG. 2C). Thus, we could derive self-renewing cell lines by permanent activation of Wnt/β-catenin with or without FGF2.

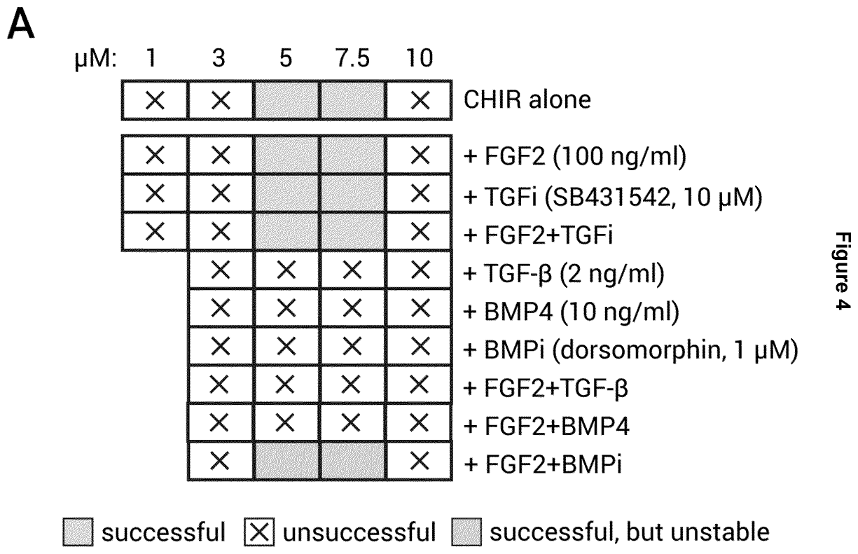

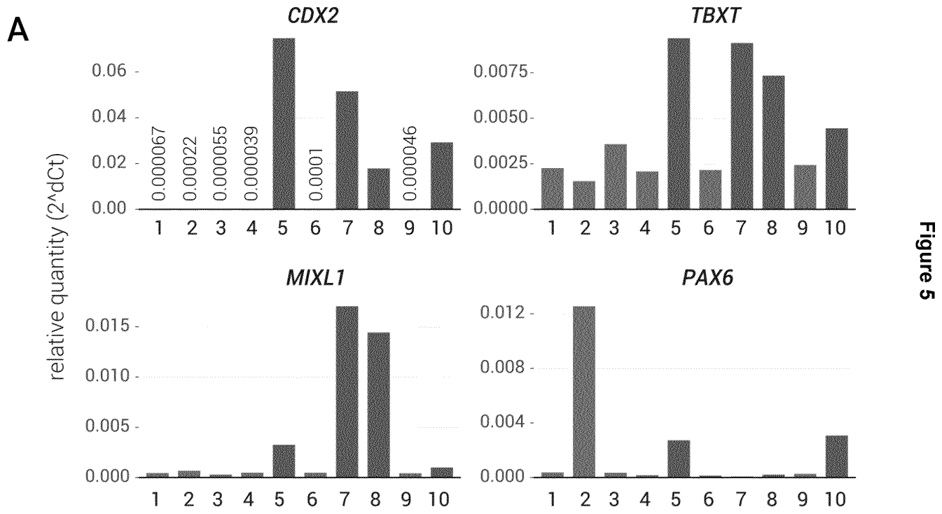

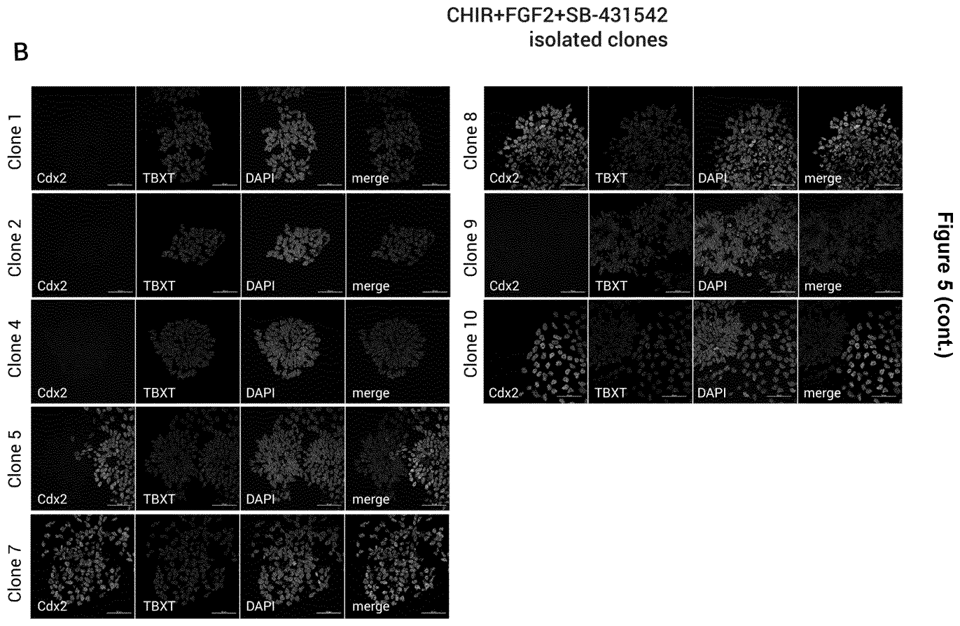

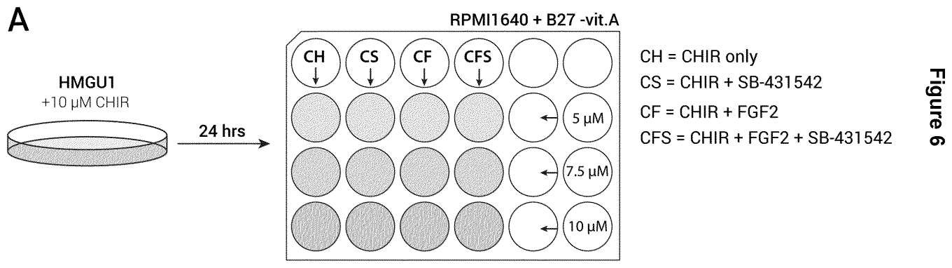

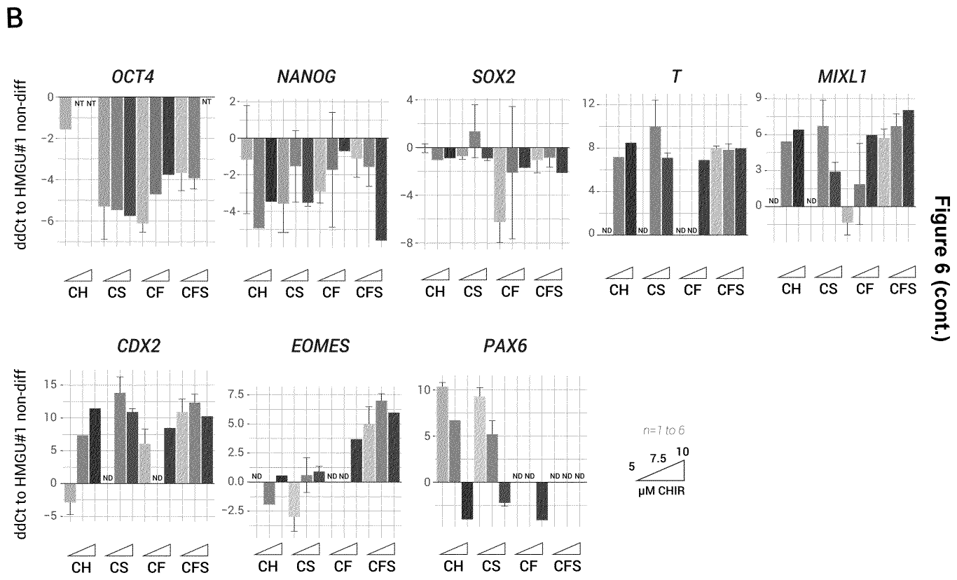

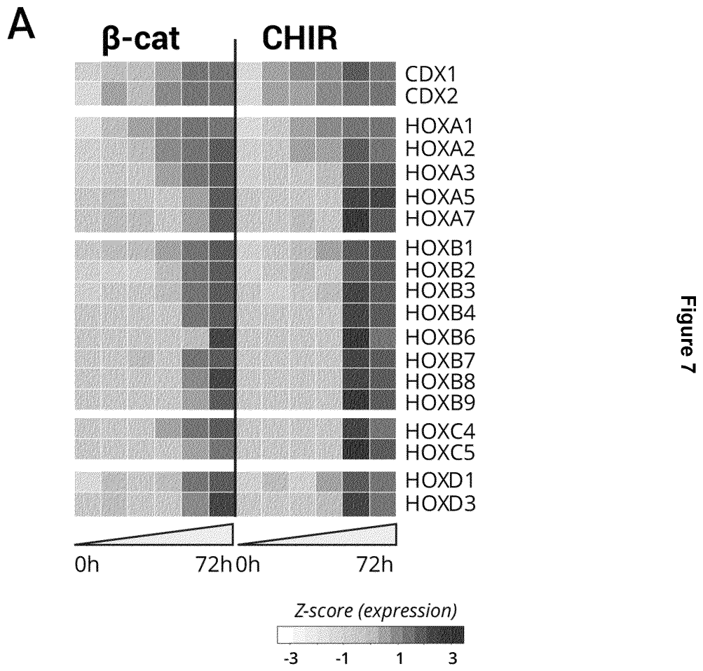

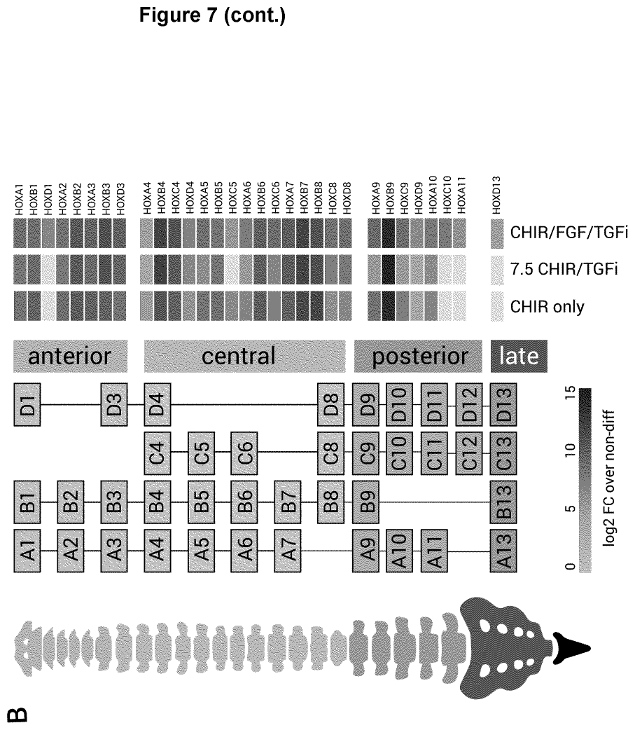

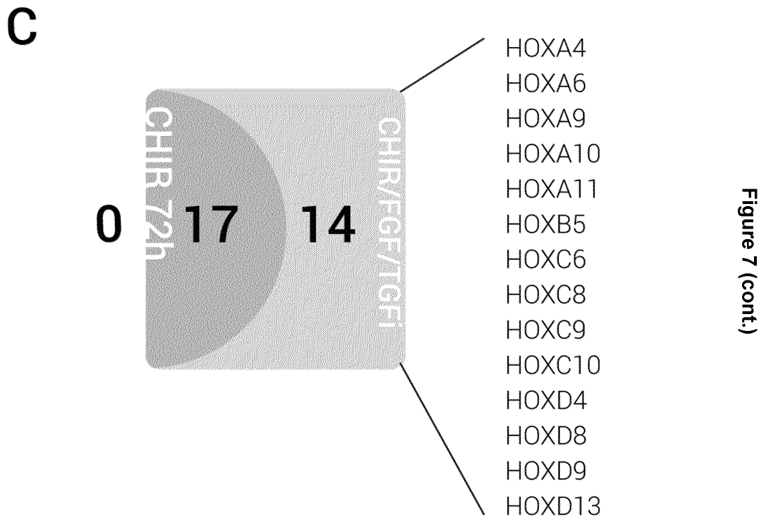

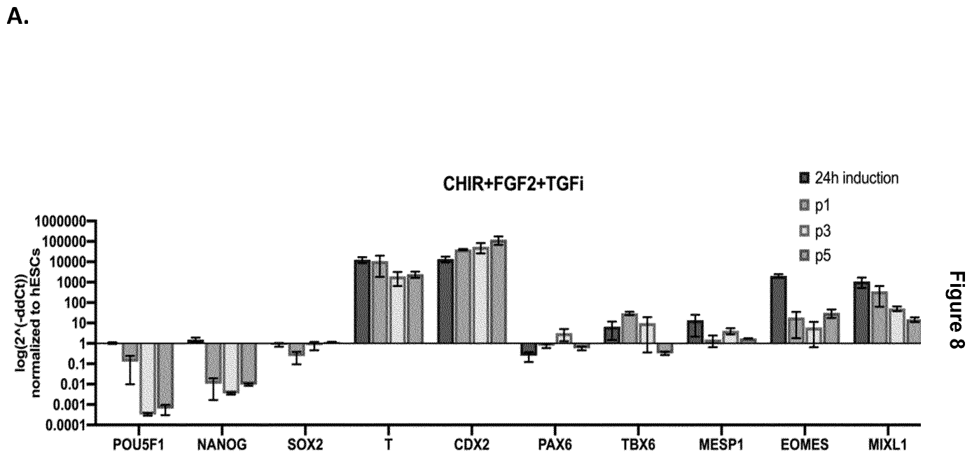

For the duration of the passaging campaign we monitored the expression of axial mesoderm and neural markers, which collectively led us to define two primary states of putative axial stem cell lines. Cell lines that were derived by the treatment of 5 μM CHIR99021 with FGF2, exhibited high levels of SOX2, TBXT, CDX2, MIXL1, EOMES and very low level or no PAX6 (e.g., FIG. 2C-E). Conversely, treatment by 5 μM CHIR99021 alone led to formation of cell lines that exhibited high levels of SOX2 in conjunction with PAX6, but no T, CDX2, MIXL1 or EOMES (e.g., FIG. 2C-E). By use of immunocytochemistry we confirmed that SOX2/TBXT/CDX2 and SOX2/PAX6 are co-expressed in the cell lines established with or without FGF2, respectively (e.g., FIG. 2C). Notably, cell lines with very similar phenotypes formed when TGF-β was inhibited, with the exception of initial expression of TBXT in cell lines produced without FGF2 (e.g., FIG. 2D,E), indicating that intermediate axial state cell lines could also be derived. Indeed, when we derived cell lines by treatment with 7.5 μM CHIR99021, we noted a stable phenotype characterized by intermediate levels of TXBT and PAX6 (e.g., FIG. 2F, G). Collectively, this indicates that the extremities of axial stem cell states which we reproduced in vitro, are characterized by mutually exclusive expression of TBXT and PAX6. Importantly, initial characterization of cell line states that were derived from human iPSCs under the same conditions demonstrated similar phenotypes, albeit the inhibition of TGF-β in 10 μM CHIR99021 was permissive for the derivation of SOX2/TXBT axial cell lines (e.g., FIG. 6). Finally, down-regulation of NANOG and shutoff of OCT4 was observed in all clones, which is in line with the restriction of their expression to pre-streak stages ref. Moreover, we noted an increase of SOX2 over extended passaging (e.g., FIG. 2E).

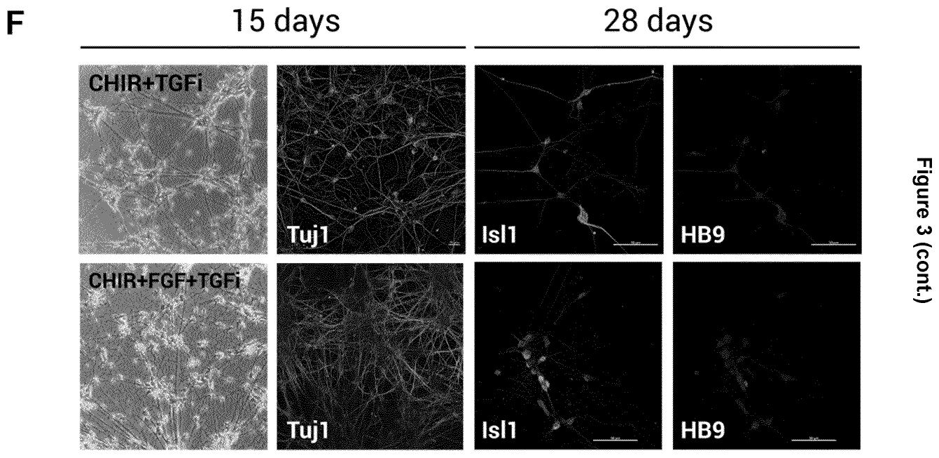

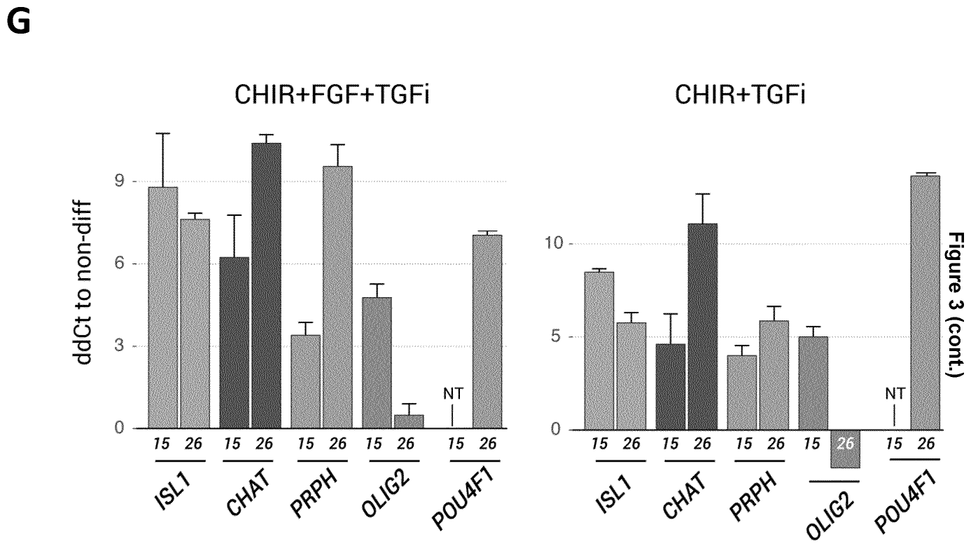

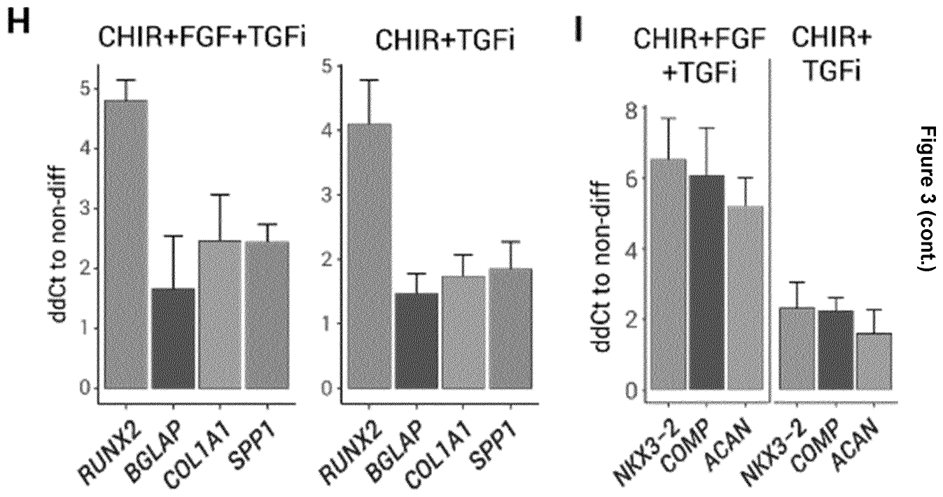

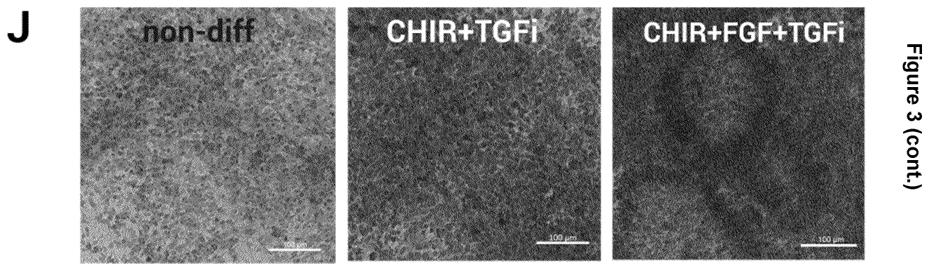

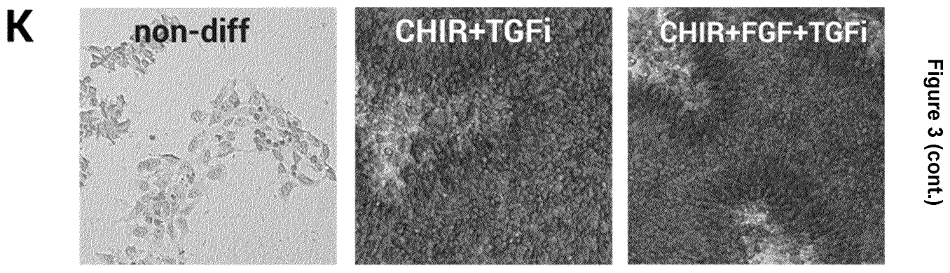

Importantly, we noted that some of the SOX2/TXBT axial cell lines exhibited heterogeneous expression of CDX2, with some colonies being uniformly positive while others negative. To further define the characteristics of CDX2 positive and negative cells, we raised clones from single cells. We noted that the clones were either CDX2-negative or contained mixed populations, and that high CDX2 expression correlated with high TBXT and MIXL1 expression (e.g., FIG. 5). Interestingly, one clone expressed PAX6 alone. Taken together these results indicate that the initial stage in the hierarchy of axial states captured by the cell lines exhibits expression of SOX2, TBXT, CDX2 and MIXL1, while the latest stage is characterized by expression of SOX2 and PAX6. We named these respective states ground- and primed-state respectively. Remarkably, both states expressed a broad repertoire of genes from the HOX cluster, which included some of the latest HOX genes in the ground state.

### Example 3: Axial Stem Cells are Hierarchical and Differentiate into Peripheral Neurons, Sclerotome and Dermomyotome

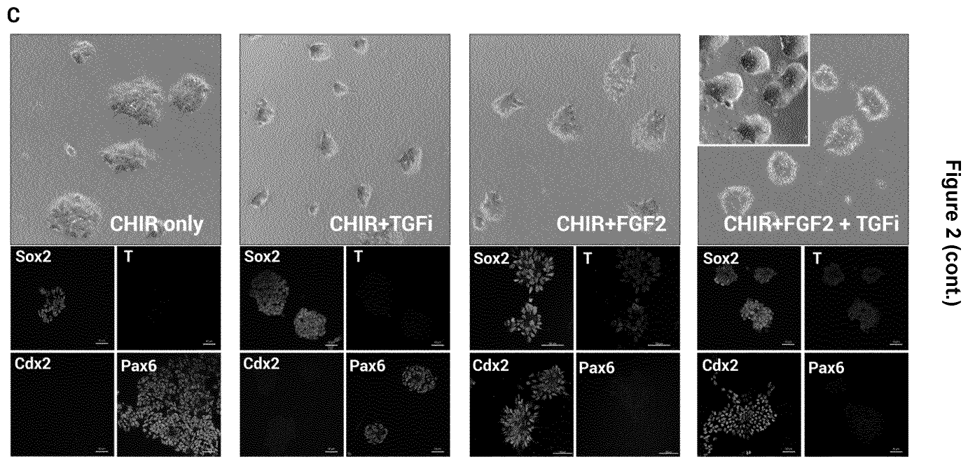

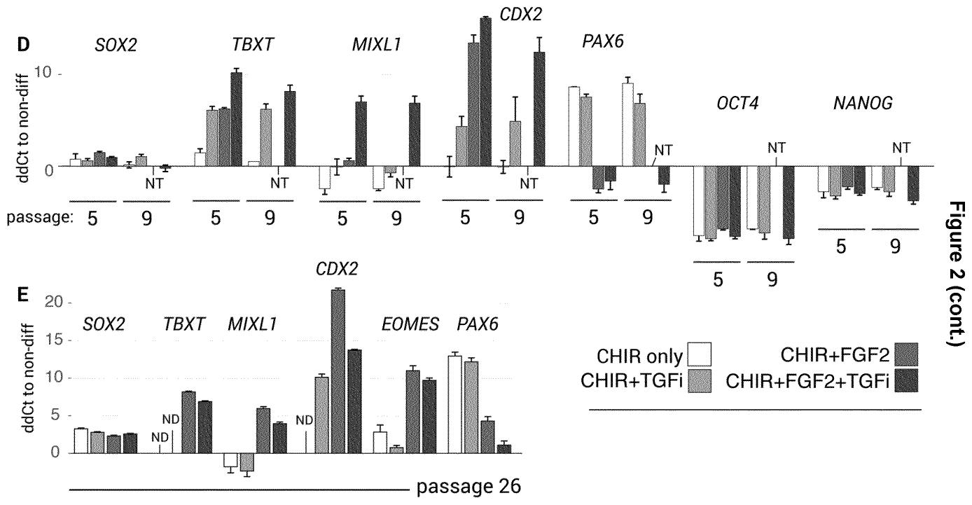

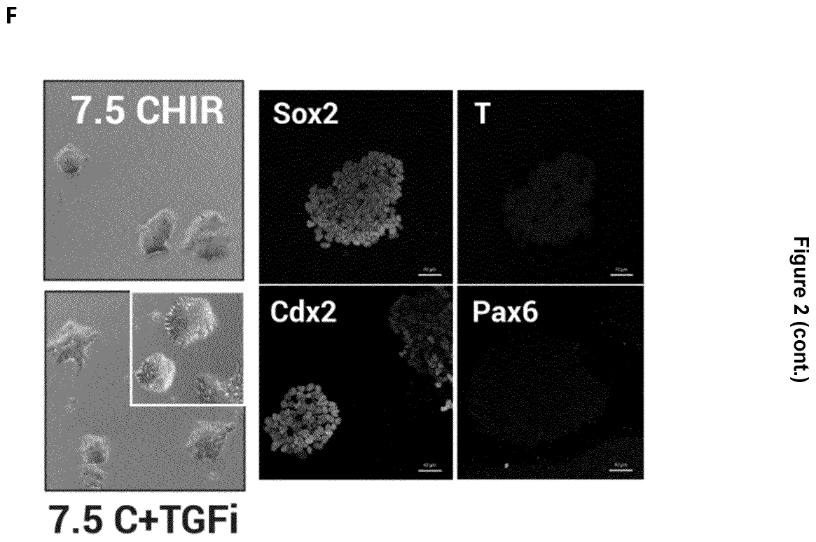

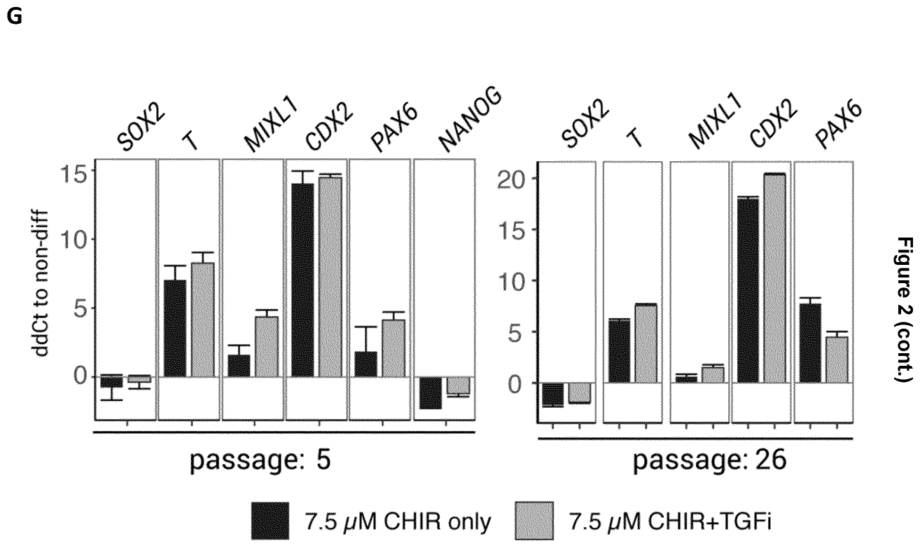

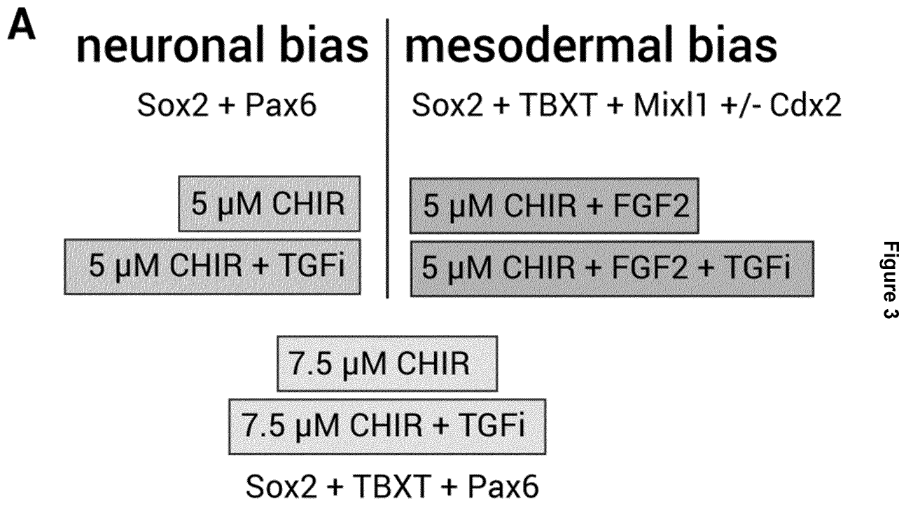

To characterize the putative axial stem cell lines, we first analyzed their hierarchy by switching off and on the FGF2 treatment. We noted that in the absence of FGF2 the ground-like axial cell lines down-regulated TBXT/CDX2 and up-regulated PAX6, while expression of SOX2 and PAX6 did not change following the addition of FGF2 to SOX2/PAX6 axial cell lines (e.g., FIG. 3A). This substantiated the premise that the SOX2/TBXT axial cell lines represent a precursor of the SOX2/PAX6 stage. Next we analyzed the ground, intermediate and primed axial stem cell states by global transcriptomes. Interestingly, despite the mutually exclusive expression of T/CDX2 and PAX6 in the ground and primed states respectively, we found that the cohort of the up-regulated genes of the primed state was almost entirely included in the ground state (approximately 90%) (e.g., FIG. 3B). This indicates that the regulation of the early axial state involves expression of a larger cohort of genes, which is possibly mediated by TBXT/CDX2/MIXL1 and/or FGF signaling. Moreover, we found that the intermediate state exhibited up-regulation of a unique cohort of comprising approximately 10% of the genes that did not overlap with the other states. Despite these differences, analysis of the enriched tissue categories indicated that the developmental correspondences of the ground, primed, and intermediate state are similar and have the potential to differentiate to neuronal and skeletal systems (e.g., FIG. 3C).

Because axial stem cells represent a developmental stage that is past the induction of the primitive streak, in order to access the neuronal potential of the ground and primed cell lines, we skipped the first set of signals that are commonly used to induce neuronal differentiation from the stage of pluripotency, including dual Smad inhibition. Remarkably, when we directly treated these cell lines by motor neuron maturation media, which included RA, SHH, BDNF and GDNF, the morphological transformation was rapid and cells developed neuronal cell morphology within 4 days in the case of the ground state and 2 days in the case of primed state. Within 15 days elaborate networks of TUJ1 positive neurons were readily apparent (e.g., FIG. 3D), and the motor neuron progenitor marker OLIG2 was highly expressed (e.g., FIG. 3E). Later, OLIG2 was down-regulated and transcription factors that are expressed by terminally differentiated motor neurons including ISL1 and HB9 as well as choline acetyltransferase (ChAT) and peripherin (PRPH) were up-regulated (e.g., FIG. 3E). Additionally, BRN3A (POU4F1), a common marker for sensory neurons, was upregulated at the latest time point of the analysis in differentiated neurons that were derived from both the ground and primed axial cell lines (e.g., FIG. 3E). Altogether, these data indicate that regardless of their state, axial stem cell lines differentiate to mature peripheral neurons, and that the ground and primed states might have different tendencies to differentiate to sensory and motor neurons, respectively. This was supported by the distinct morphologies of neuron networks that formed.

According to the same premise, skipping the early stages of induction from pluripotent state, should allow axial stem cell lines to differentiate to sclerotome lineages. Indeed, when we treated ground and primed state axial stem cells by an activator of the sonic hedgehog pathway and medias that promote osteocyte and chondrocyte maturation, we found that differentiated cells derived from both states exhibited definitive characteristics of respective cell types. This included the up-regulation of RUNX2, the main transcription factor required for specification of the osteoblast lineage (Komori, 2010), and its target genes that are specific to osteocytes—osteocalcin (BGLAP), osteopontin (SPP1) and collagen 1a1 (COL1A1) (e.g., FIG. 3F). Similarly, we noted up-regulation of chondrocyte markers including NKX3-2, a transcription factors that activates genes that are expressed in cartilage tissues, including oligomeric matrix protein (COMP) and aggrecan (ACAN) (e.g., FIG. 3G). Notably, the up-regulation was stronger for cells differentiated from the ground state lines, indicating that the tendency to produce cartilage is higher in ground state axial stem cell lines. Finally, staining of calcium deposits by Alizarin red and of sulphated proteoglycans by Alcian blue confirmed the differentiation of osteocytes and chondrocytes (e.g., FIG. 3H-I). We therefore conclude that the two states of axial stem cells can produce the sclerotome, but that the propensity of differentiation differs according to the developmental stage.

To induce dermomyotome differentiation skeletal muscles we applied a recently developed protocol for sensorimotor organoids (Pereira et al, 2019, CellPress SneakPeek), starting the process with ground and primed axial cell lines. At day 16 we noted significant up-regulation of PAX3, PAX7 and MYOD1, indicating skeletal muscle formation.

Finally, we transplanted ground and primed state axial stem cell lines into immunodeficient mice, and in contrast to undifferentiated hESCs, in 100% of the transplantation cases we did not detect any teratoma formation. Taken together the evidence of differentiation to peripheral neurons, sclerotome and dermomyotome but not to teratomas, support the classification of the axial-like ground and primed state cell lines as equivalent to human embryonic axial region precursors.

Discussion

SOX2 has a critical role in the maintenance of embryonic and neural stem cells (NSCs) (Boyer et al., 2005; Avilion et al., 2003; Graham et al., 2003; Pevny and Nicolis, 2010), and is also expressed in the multipotent progenitors of the peripheral nervous system, for example, axial progenitors, residing in the NSB, CLE and CNH (Henrique et al., 2015) or neural crest-derived sensory neuron progenitors (Cimadamore et al., 2011). We show here that the expression of SOX2 can be kept high as in human ESCs by constant activation of the Wnt/β-catenin pathway in conjunction with cell passaging that begins 24 hours following activation of the pathway. This enabled us to untangle the roles of Wnt/β-catenin and FGF in the regulation of SOX2, PAX6 and TBXT in axial progenitor differentiation. Despite TBXT being thought to form a positive feedback loop between Wnt/β-catenin and FGF signaling that is necessary for establishing and maintaining axial progenitors (Garriock et al., 2015; Gouti et al., 2014; Koch et al., 2017; Turner et al., 2014; Martin and Kimelman, 2010), our results show that Wnt/β-catenin alone is sufficient for maintaining the expression of SOX2 in axial progenitors. Instead, FGF and Wnt/β-catenin signaling together are necessary for establishing and maintaining a state of axial progenitors that co-expresses TBXT and SOX2. Nevertheless, TBXT is transiently expressed following activation of Wnt/β-catenin and passaging, and FGF could be produced endogenously (e.g., FIG. 1F), which indicates that TBXT and FGF are transiently involved in the establishment of all forms of axial progenitors. This is supported by the unidirectional conversion of the SOX2, TBXT→SOX2, PAX6 axial state by Wnt/β-catenin alone. Importantly, if hPSCs are not passaged following Wnt/β-catenin stimulation, SOX2 is quickly down regulated, which prohibits the derivation of axial progenitors altogether. Thus we conclude that Wnt/β-catenin and passaging promote TBXT and SOX2 axial ground state in the presence of bFGF, or a primed state that coexpresses SOX2 and PAX6 when only Wnt/β-catenin signaling is active (e.g., FIG. 3K).

It has been previously shown that PAX6 is essential for neuroectoderm differentiation of human fetuses and human PSCs, as apposed to mouse neuroectoderm where Sox1 is the earliest marker (Zhang et al., 2010). Based on axial progenitor differentiation, our results indicate that PAX6 is the earliest neurogenic factor in progenitors of the human peripheral nerve system differentiation. Importantly, FGF2 has been shown previously to suppress PAX6 during human PSC differentiation to neurons (Greber et al., 2011). Moreover, MEK-ERK signaling has been implicated in mesoderm differentiation and expression of TBXT (Yao et al., 2003). This indicates that FGF2 has a dual role in inhibiting human axial progenitor differentiation to the primed state (e.g., FIG. 3K). Part of the mechanism may involve switching the enhancer regulated in SOX2 from N1 in undifferentiated ESC to Wnt/FGF-dependent N2 enhancer in the axial state (Takemoto et al., 2006; Kondoh and Takemoto, 2012).

Beyond mechanisms of early differentiation, we derived for the first time self-renewing stem cell lines that correspond to the respective stages of axial progenitors, ground and primed, from both hESCs and iPSC lines. The stem cell lines exhibited stable undifferentiated phenotype for dozens of passages, and we noted that TGF-β inhibition by SB-431542 reduced spontaneous differentiation and maintained the lines as more compact colonies. Importantly, single cell cloning of ground state AxSC line that was treated by SB-431542 produced mixed or CDX2-clones, indicating that at the root state, AxSCs expresses CDX2. It is possible that the clonal patterns of CDX2 is due to heterogeneity in the expression of the late HOX gene HOXD13, which is known to negatively regulate CDX2 (Young et al., 2009; Amin et al., 2016). Finally, it is likely that FGF8 can replace FGF2 in the derivation of AxSC lines (Lippmann et al., 2015). Taken together, the pathways that regulate differentiation of human axial progenitors can be utilized to create ground and primed state AxSC lines by passaging the cells when the expression of SOX2 coincides with TBXT following activation of Wnt/β-catenin with or without FGF2.

Our evaluation of the differentiation potential of ground state and primed AxSC lines showed that both types differentiate rapidly, within as few as 48 hours, into complex networks of neurons. Therefore, the neural commitment represents a default pathway of differentiation for AxSCs, albeit it took the cells approximately 10 days to express high levels of peripheral and motor neuron markers, which indicates that the maturation and diversification of peripheral neurons may involve additional signals. It is interesting that the morphologies of neural networks were different between ground and primed state AxSC lines. The higher expression of PRPH in neurons derived from ground state AxSC might indicate a higher propensity to generate sensory neurons relative to primed state AxSCs which also gave rise faster to dense neuron networks. The idea that neuronal differentiation is a default pathway for AxSCs is supported by the fact that protocols of 15 and 15-30 days were required for the cells to become sclerotome i.e. chondrocytes and osteocytes, and dermomyotome i.e. muscle fibers, respectively. Despite that other studies reporting previously on the differentiation human NMPs progenitors to motor neurons (Gouti et al., 2014; Lippmann et al., 2015; Denham et al., 2015; Verrier et al., 2018) and one study reporting the differentiation of early muscle cells (Gouti et al., 2014) , to our knowledge AxSC represent the first and only type of multipotent clonal cell line that is capable of giving rise to sclerotome, dermomyotome and peripheral neurons.

The ordering of the expression of HOX genes along the body axis in mouse embryos and the regional activation of specific HOX genes in body segments of invertebrates is thought to reflect a deterministic mode of HOX gene activation in mammals as well. Remarkably, we found that the repertoire of HOX genes expressed by AxSC lines is very broad, and in the ground-state included some of the latest posterior HOX genes. This could indicate a permissive mode of HOX gene ordering along the axis in mammals that operates by first activating in parallel all or majority of HOX paralog genes in AxSCs, and later fixing their positional identity by secondary signals. In line with this explanation are findings made recently by Gouti at al. who noted progressive activation of posterior HOX genes in transient NMPs from mouse embryos, and by Lippmann et al. who showed that retinoic acid fixes the positional identity of the progeny of NMPs. The ability to create single clones from ground state AxSC, and their differentiation to primed state AxSCs which do not express late HOX genes, provides an important platform to interrogate the mechanisms underlying positional precision of HOX gene patterns in mammals.

Production of human AxSC lines could be highly advantageous for applications in research and medicine. First, application of such lines or their progeny may increase the safety of cell therapies in the peripheral nervous system, skeletal muscle or skeletal tissues by circumventing risks of cellular impurities. These could be residual undifferentiated PSCs that bear the risk of forming teratoma tumors (Drukker, 2012) or differentiated and intermediate cell types of other tissues. Importantly, we found no evidence that ground or primed state AxSCs can give rise to teratomas. Second, the a priori developmental restriction of AxSCs could make the derivation of specific types of cells of interest, such as motor neurons, more coherent, uniform and faster, and as a result less costly and simpler for manufacturing of therapeutic cells. Therefore, AxSCs can also become a tractable tool for creating cell models for diseases affecting the peripheral nervous system such as amyotrophic lateral sclerosis (ALS), and for drug development and toxicity testing using progeny of AxSCs. Third, AxSCs could become a benchmark for understanding regulation of development, function and evolution of the human peripheral nerve system and additional cell types that are born in the axial region. In this regard, we anticipate that induced AxSCs (iAxSCs) could be derived directly from somatic cells by over expression of key transcription factors described here, much like the case of induced neurons and iPSCs.

Conclusions

The derivation of NSCs from embryos and ESCs marked the beginning of a new era in the study and therapy of the central nervous system. The derivation of AxSC lines complements these important findings by providing systems to study and create therapies for diseases and traumas affecting the sensory and motor peripheral nervous system as well as of the skeletal muscle, including ALS and muscular dystrophies.

### Example 4: Derivation of Stem Cell Lines with Characteristics of Axial States

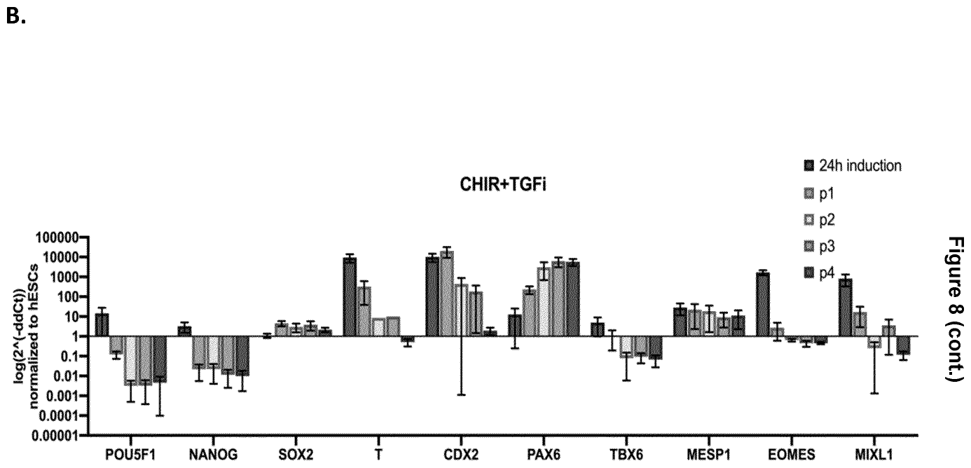

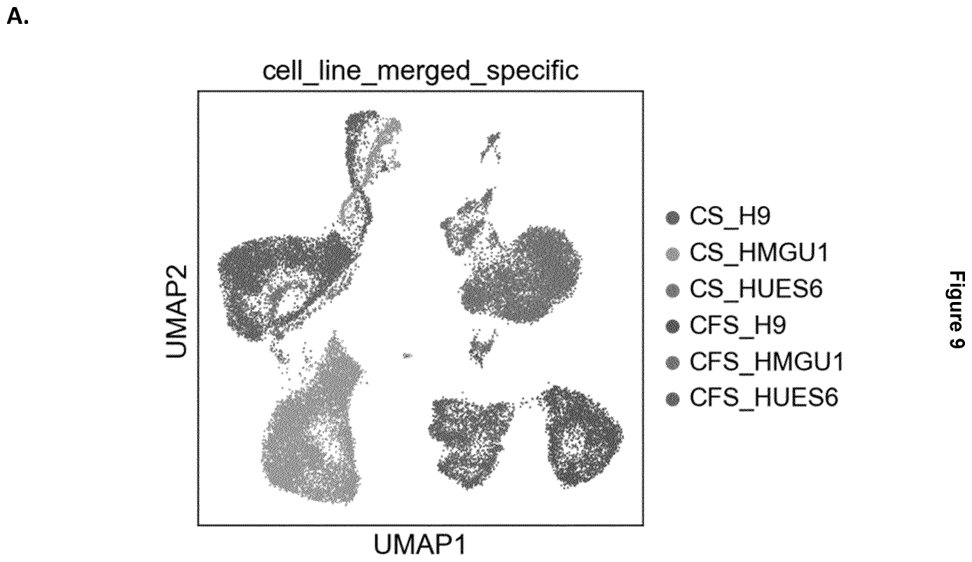

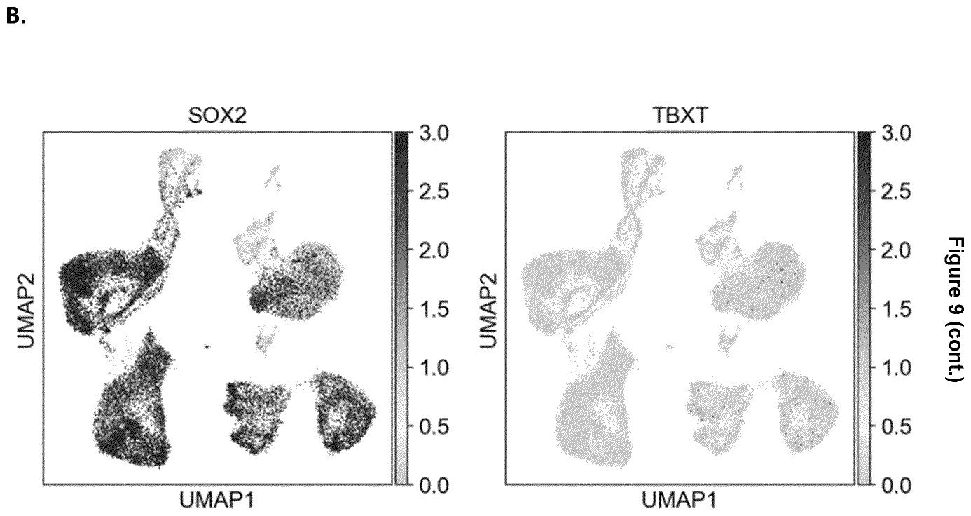

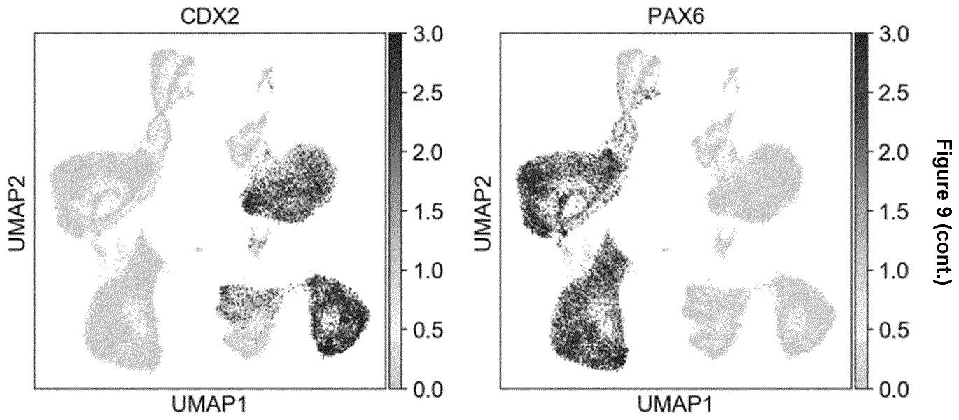

CHIR+TGFi and CHIR+FGF2+TGFi treatments enabled derivation of stable cell lines, therefore, additional derivations were performed by using those conditions to check expression trend of markers genes described previously in first passages during establishment process. Similar to previous results for passage 5, 9 and 26, axial progenitor markers (SOX2, TBXT and CDX2) were detected at high level in first passages by CHIR+FGF2+TGFi (CFS) treatment (FIG. 8A), while CHIR+TGFi (CS) treatment led to gradual decrease of axial progenitor markers conversely to PAX6 which was gradually increased (FIG. 8B). Any of the lines did not express or expressed at very low level of mesodermal markers.

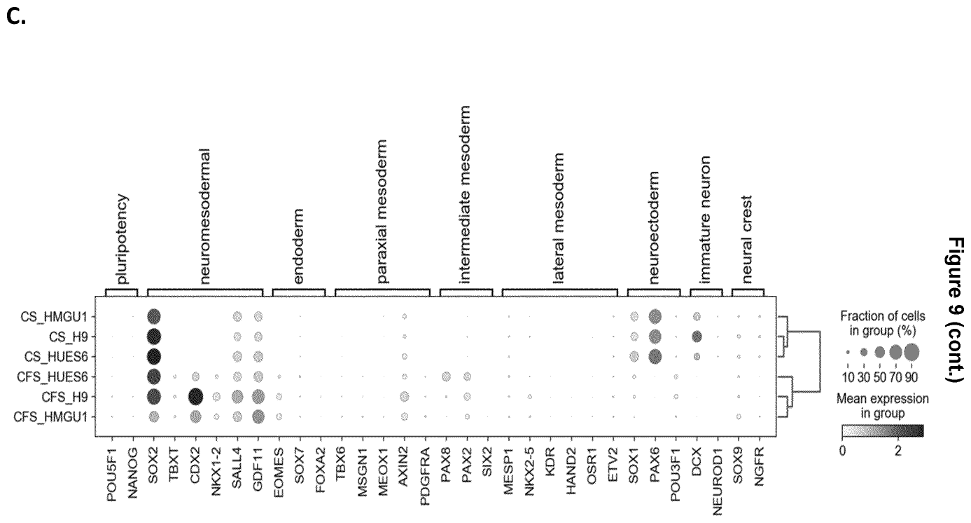

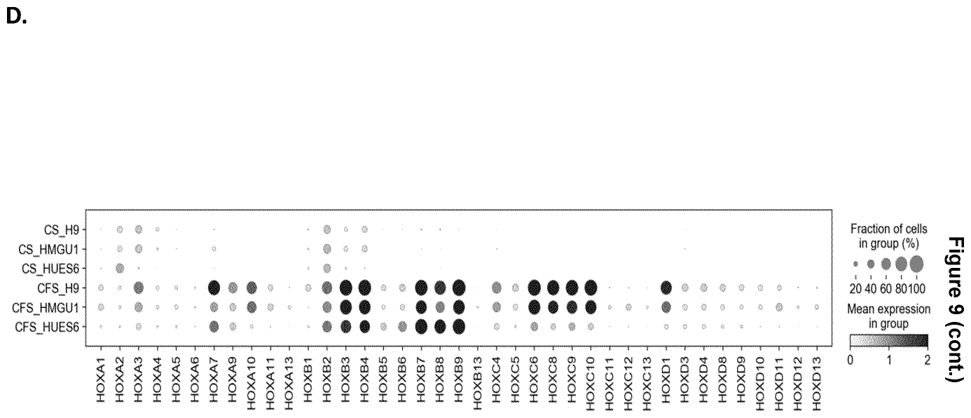

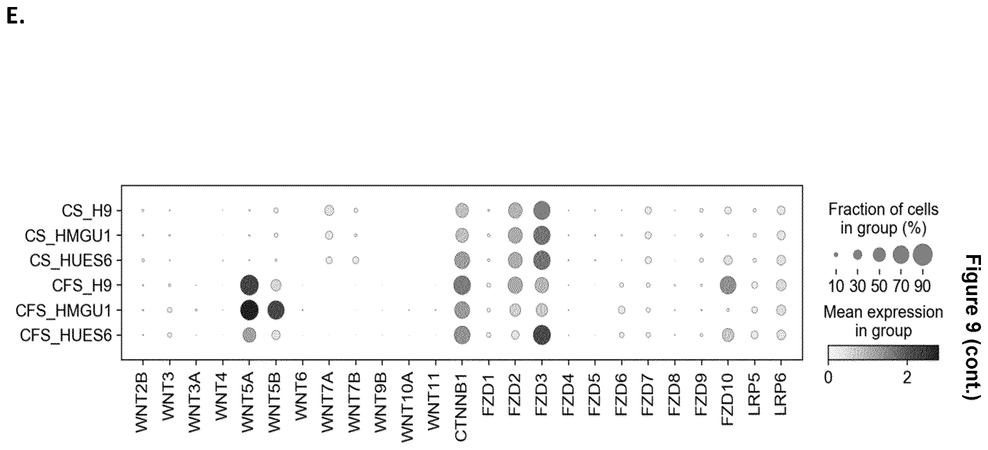

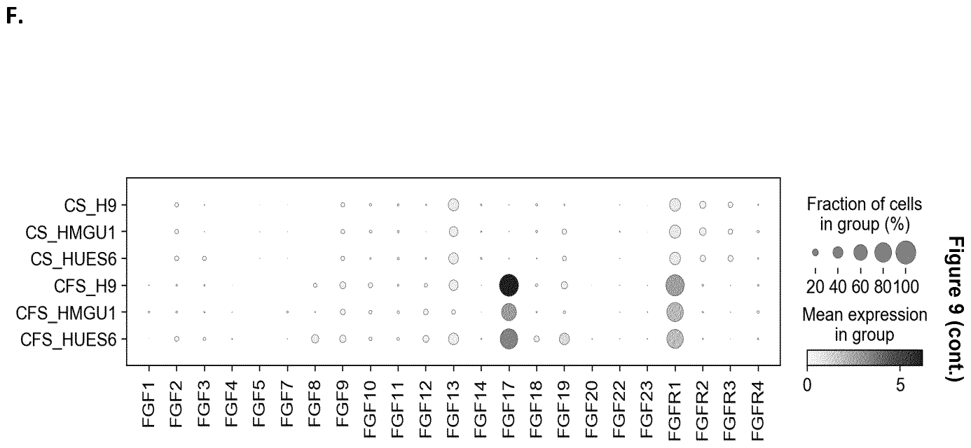

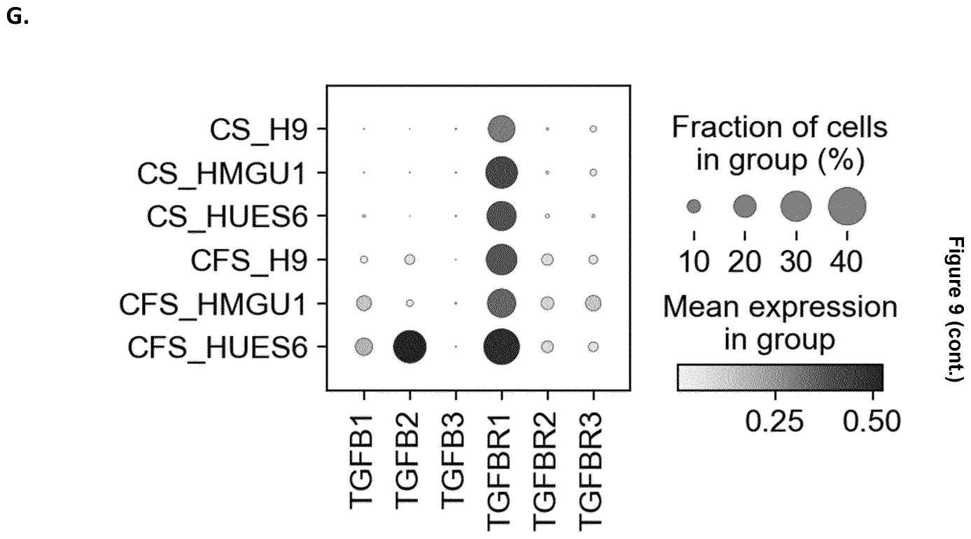

To transcriptionally characterize axial stem cell lines precisely, we performed single-cell sequencing of H9, HUES6 and HMGU1 derived axial stem cells (FIG. 9A). Axial progenitor markers were detected as heterogeneously expressed in CFS lines except for the line derived from H9 (FIG. 9B). In CFS_HMGU line, expression of SOX2 overlaps CDX2. In CFS_HUES6 line, however, both CDX2− and CDX2+ cells express SOX2. Detection of low TBXT expression might be related to technical limitations because its expression is detected in sequenced samples by qPCR. In any CS lines, TBXT and CDX2 were not expressed confirming previous results and SOX2 overlaps PAX6. Next we analyzed expression of pluripotency, neuromesodermal (axial progenitor) and lineage specific markers (FIG. 9D) and found that CFS lines express neuromesodermal genes substantially while CS lines express neuroectodermal genes.

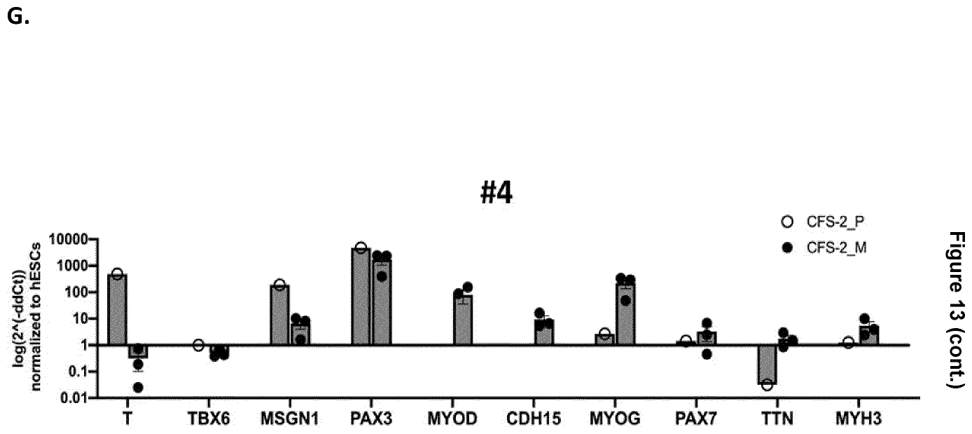

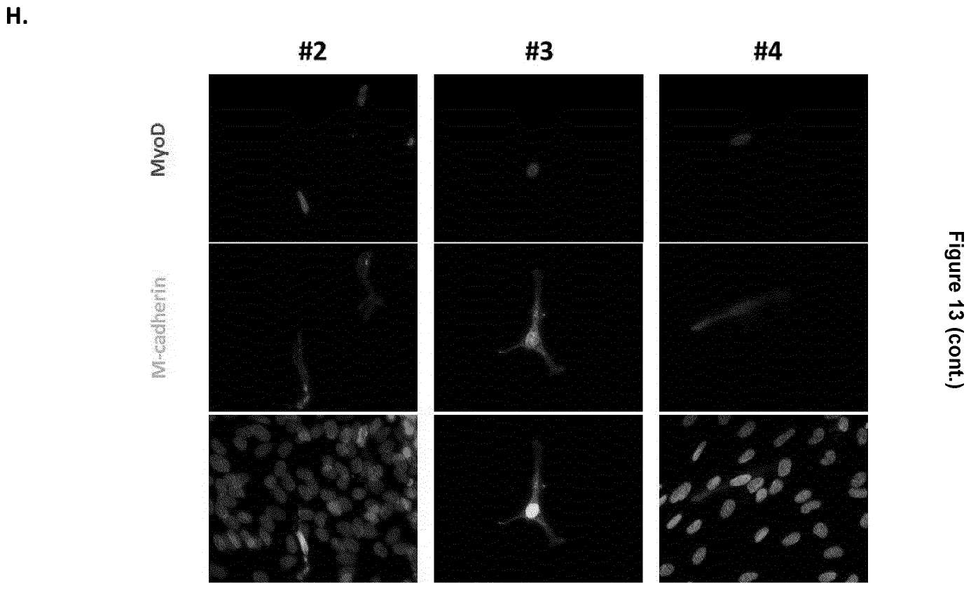

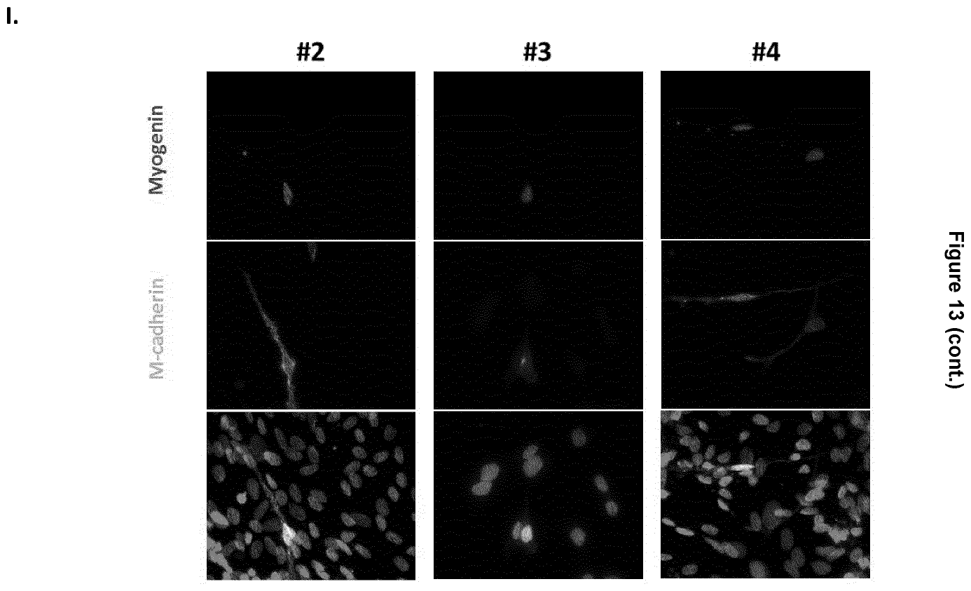

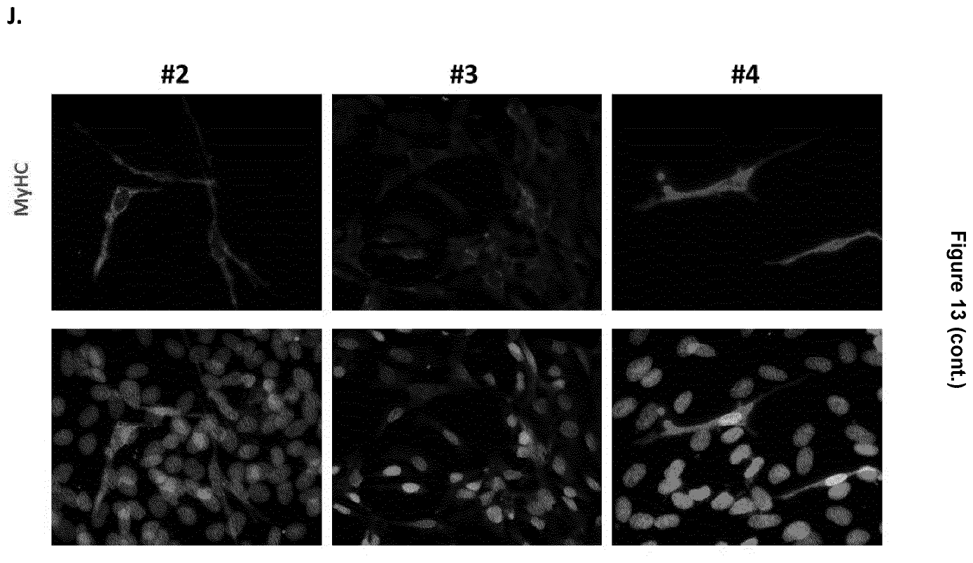

In addition to results from RNA-sequencing (FIG. 14B), we analyzed expression of HOX genes at single-cell level and noted that CFS lines have a broad range expression of HOX genes from anterior to posterior, whereas CS lines are delimited by anterior HOX genes such as HOX1-4 (FIG. 9D). We examined in all lines key signaling pathways important for axial elongation (FIG. 9E-J). Even though WNT gene and WNT receptor gene expressions vary in cell lines, CTNNB1 (B-catenin) which is downstream gene of WNT signaling pathway is highly expressed in all lines (FIG. 9E). CFS lines are detected as expressing mainly FGF17 and FGFR1, however CS lines have also low expression of FGF13 and FGFR1 (FIG. 9F). We noted that members of TGFb (FIG. 9G) and NOTCH (FIG. 9H) signaling pathways are activated in all lines in a similar manner except for NOTCH ligands which were found higher in CS line (FIG. 9H), while retinoic acid (FIG. 9I) and BMP (FIG. 9J) signaling could be more important for CFS lines.

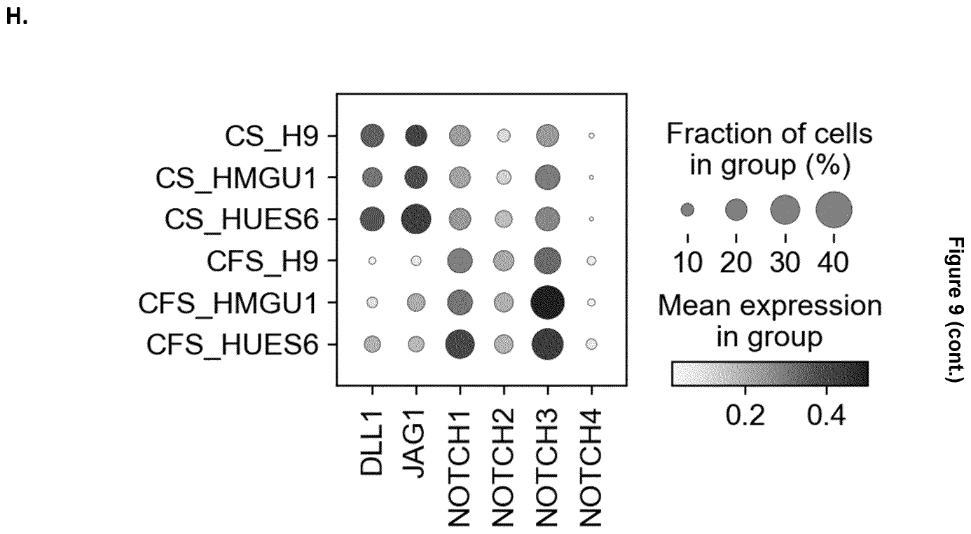

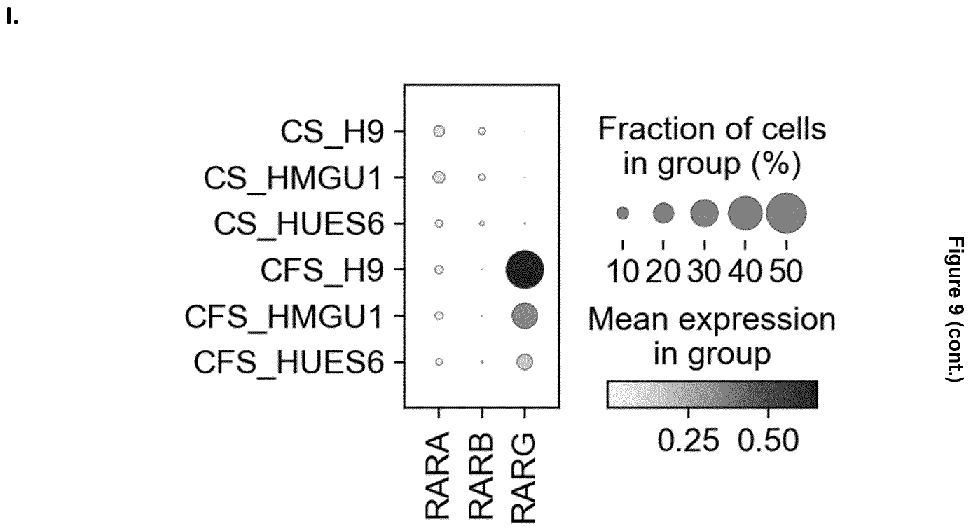

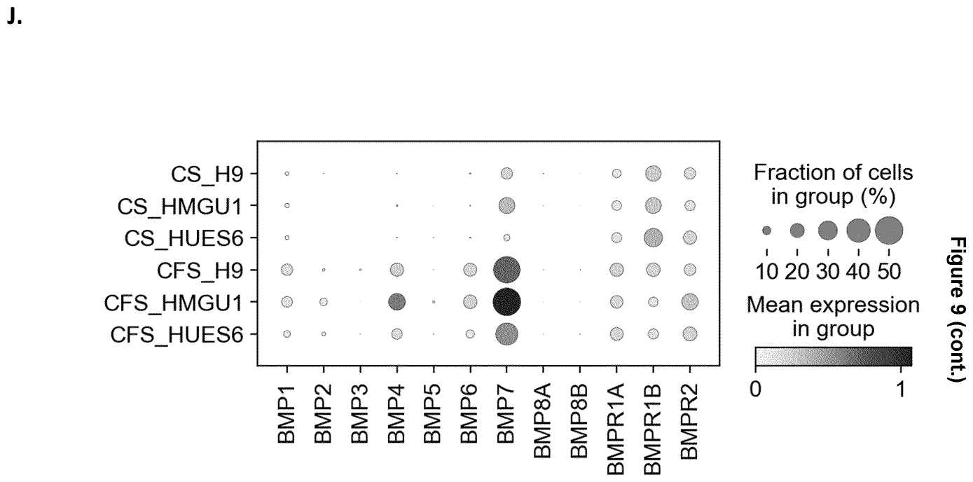

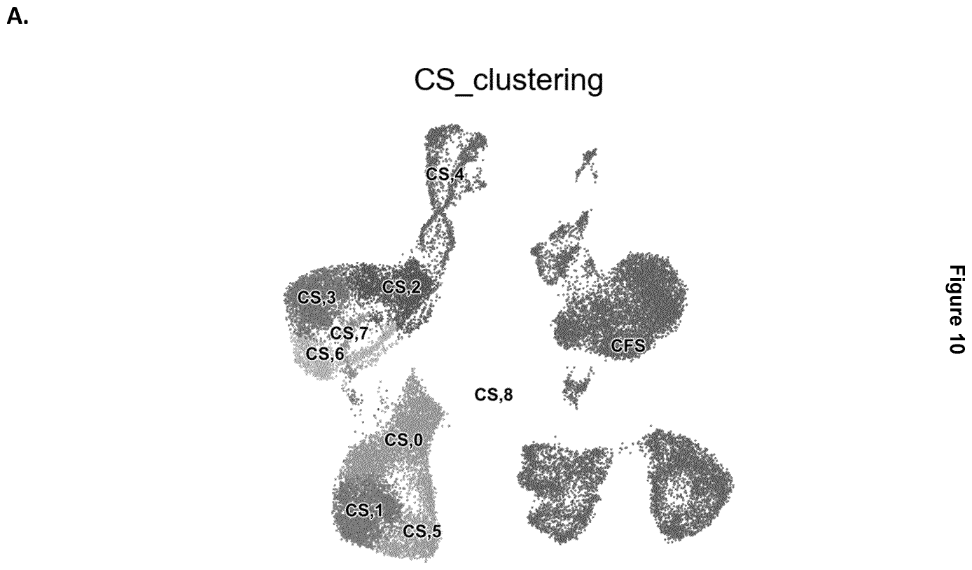

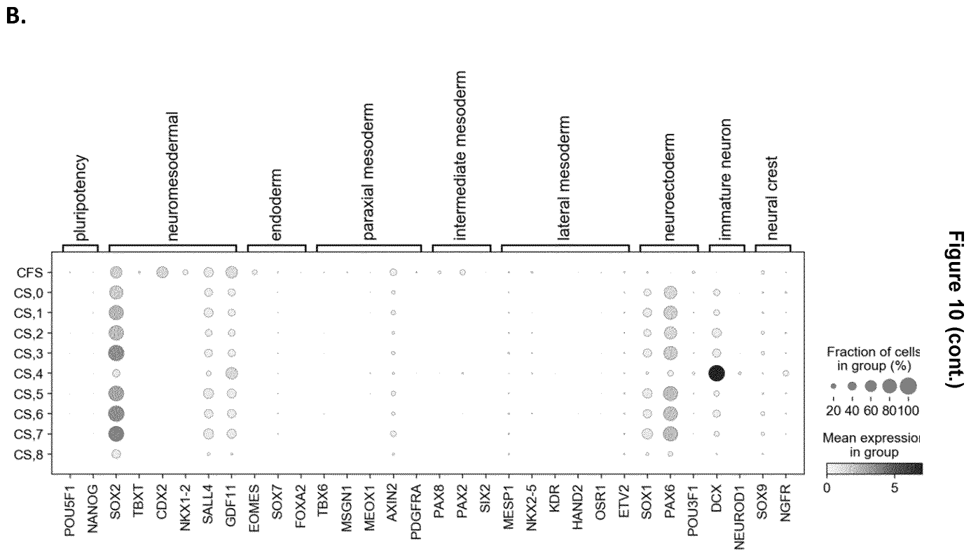

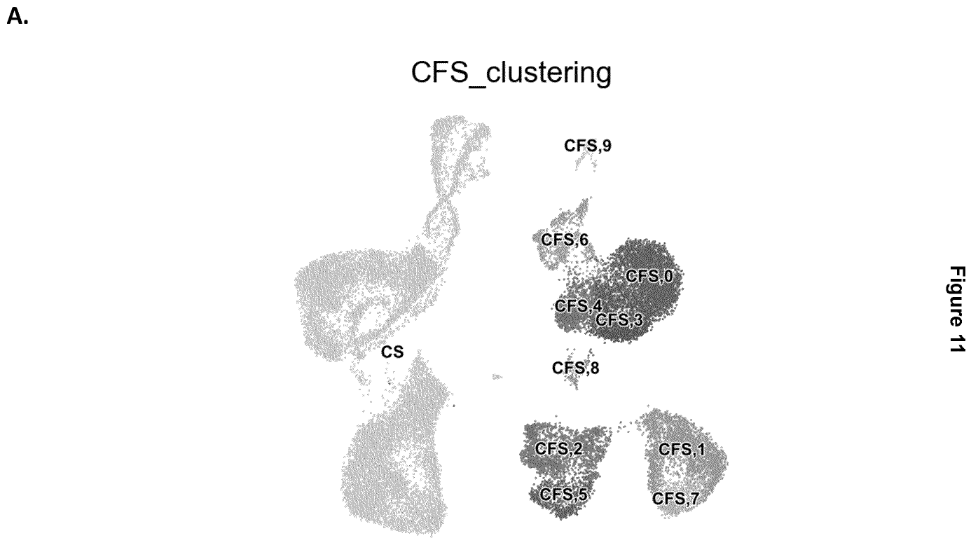

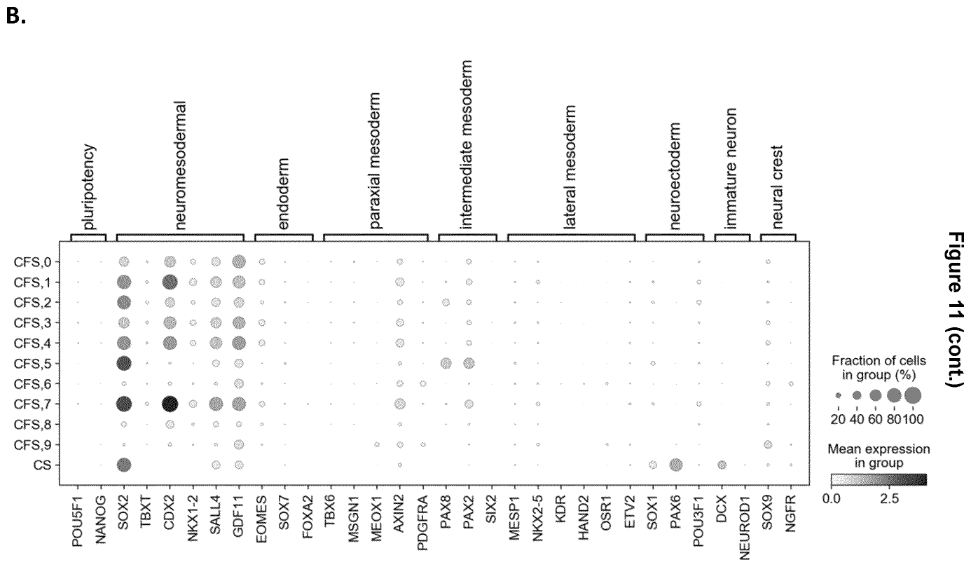

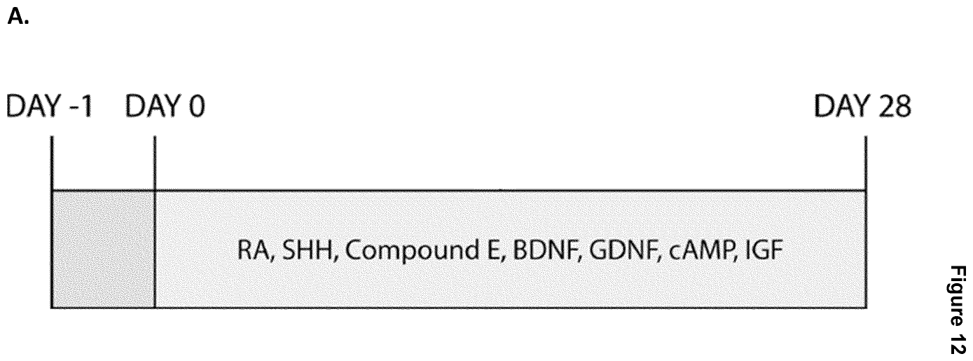

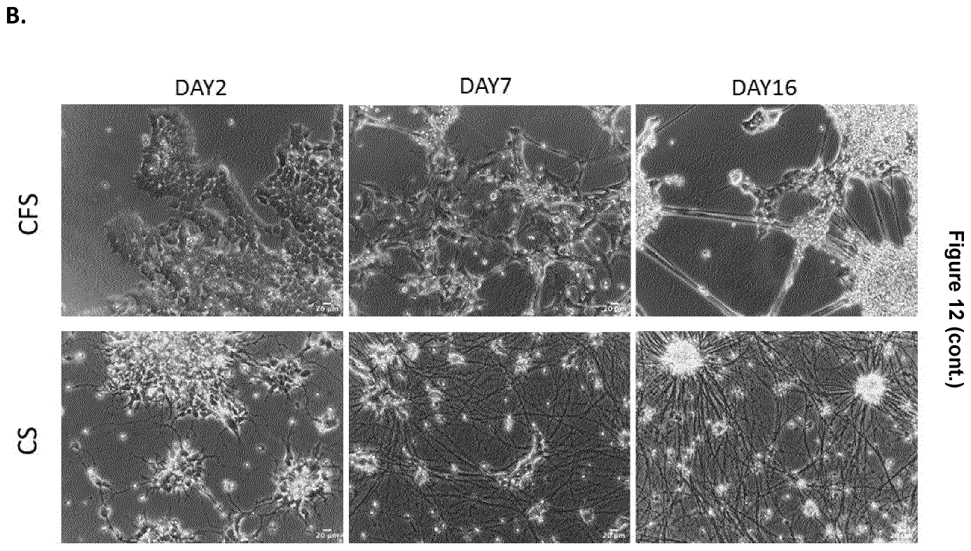

Next we analyzed clusters of both CFS and CS lines (FIGS. 10, 11) to assess if heterogeneity in SOX2, CDX2 and PAX6 is a result of differentiation of axial stem cells to lineage-committed cells. Out of 8 CS clusters, CS,4 (FIG. 10A) where SOX2 expression is relatively low (FIG. 9, FIG. 10B) highly express DCX which is immature neuron marker could be an indication of differentiation to neural lineage. In CFS clusters (FIG. 11A) we found one cluster as SOX2+ CDX2− (CFS, 5) where some of the intermediate mesoderm markers are slightly upregulated. We did not detect any distinct pattern of lineage-specific markers in between SOX2+ CDX2+ clusters and SOX2− CDX2− clusters except for CFS,9 where neural crest marker is upregulated (FIG. 11B).

### Example 5: Axial Stem Cells are Hierarchical and Differentiate into Peripheral Neurons, Sclerotome and Dermomyotome

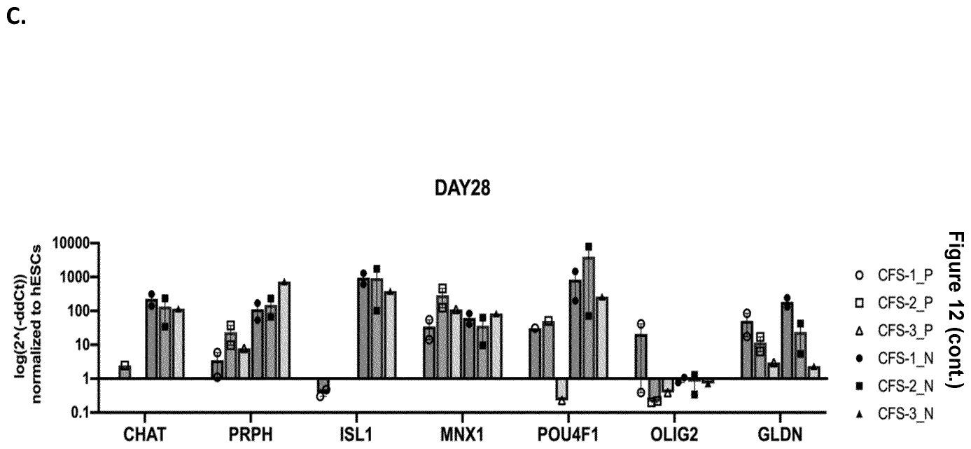

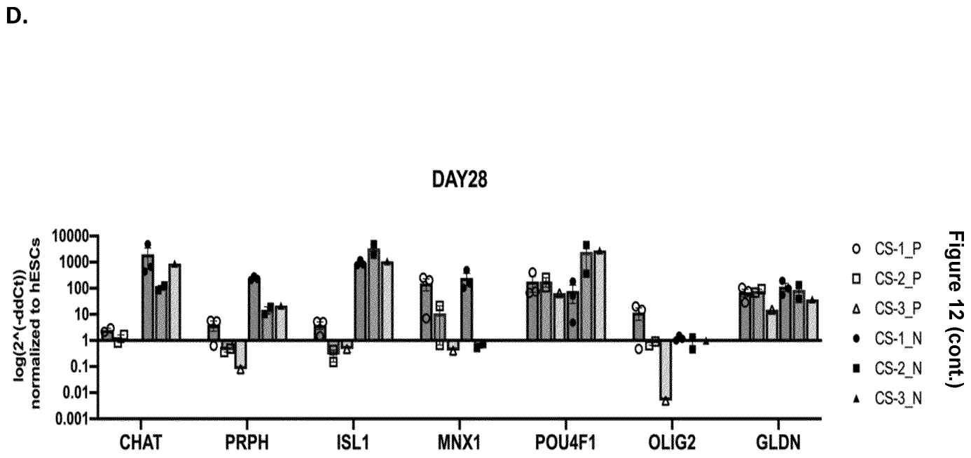

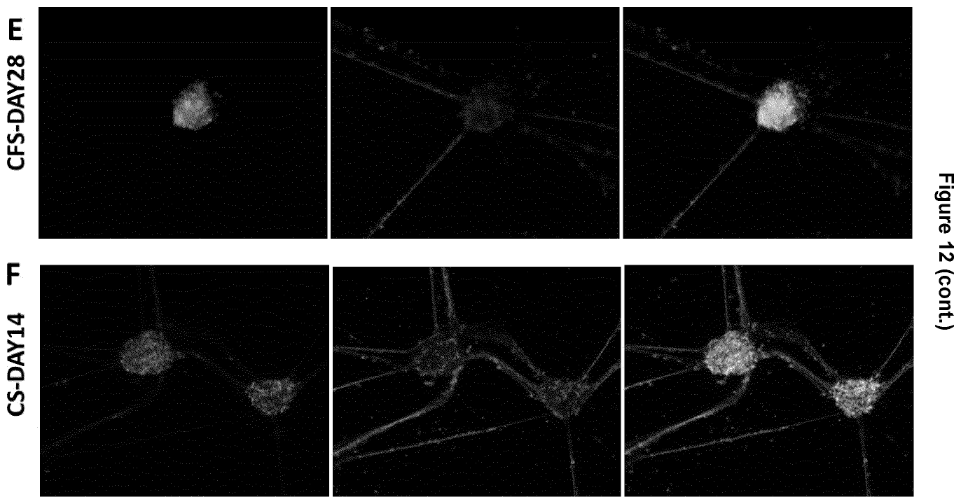

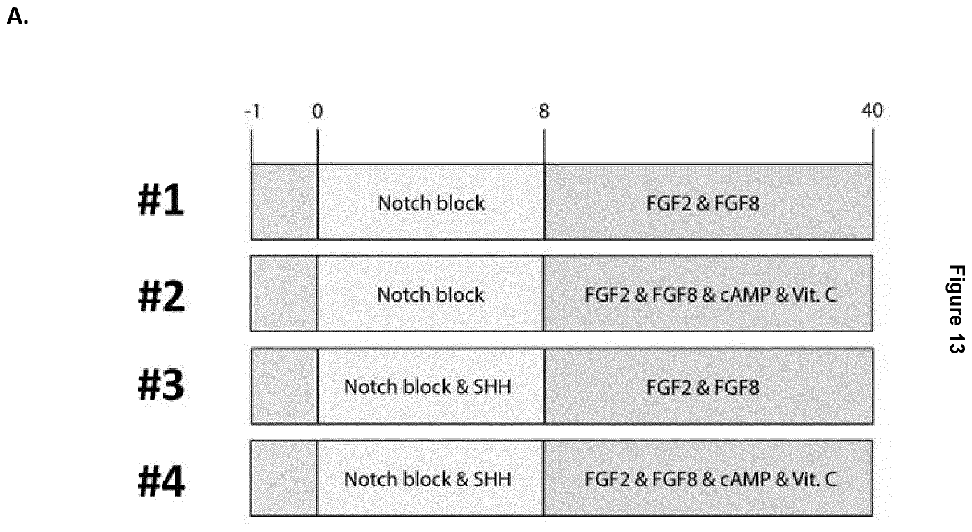

We sought to investigate capability of axial stem cells to produce peripheral neurons therefore we performed motor neuron differentiation by using medium including a group of cytokines (FIG. 12A) to mimic spinal cord development and spinal cord neuron specification based in literature REF. Based on cellular morphology during the differentiation we observed that two states of axial stem cells show different speed to develop neural morphology which was 2 days for CS and 7 days at least for CFS cells (FIG. 12B). We noted cellular heterogeneity in CFS differentiation from DAY16 onwards. Based on gene expression analyses, peripherin (peripheral neuron marker), ISL1 and CHAT (mature neuron markers) were upregulated in all experiments while OLIG2 (motor neuron progenitor marker) was not detected in any (FIG. 12C-D). Strikingly, CFS and CS differentiations showed different patterns for MNX1 and POU4F1 expression which are motor and sensory neuron markers respectively. Both transcription factors were detected in DAY28 differentiated cells from CFS derivations (FIG. 12C-E), while CS-derived cells express MNX1 at DAY14 (FIG. 12F) however either MNX1 or POU4F1 at later timepoint (DAY28) except for differentiated cells from CS-1 derivation which shows expression of both transcription factors (FIG. 12D) similarly to CFS differentiations.

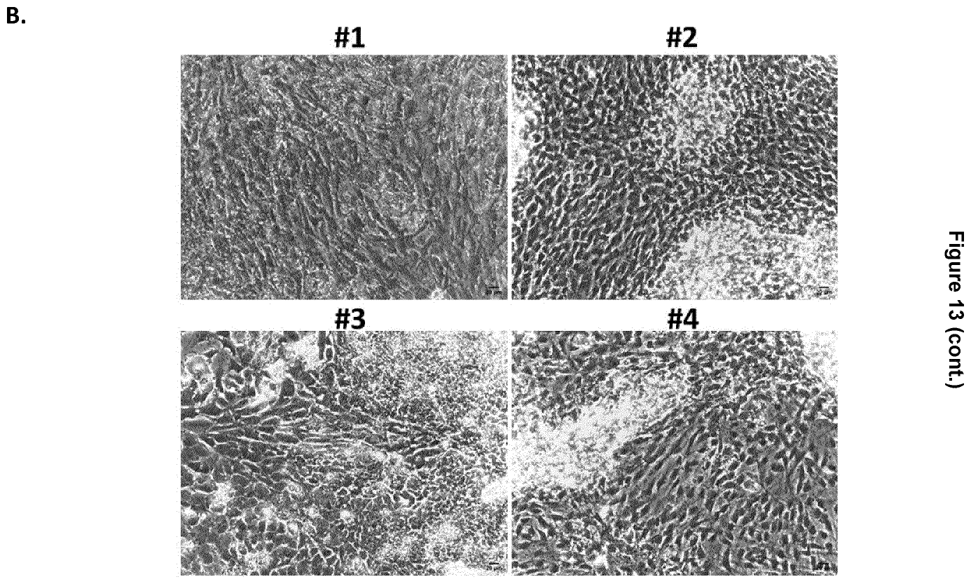

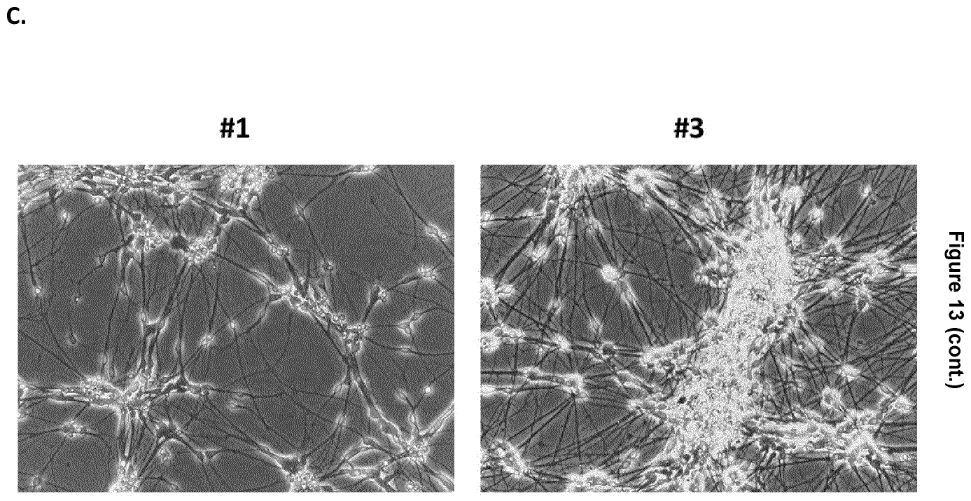

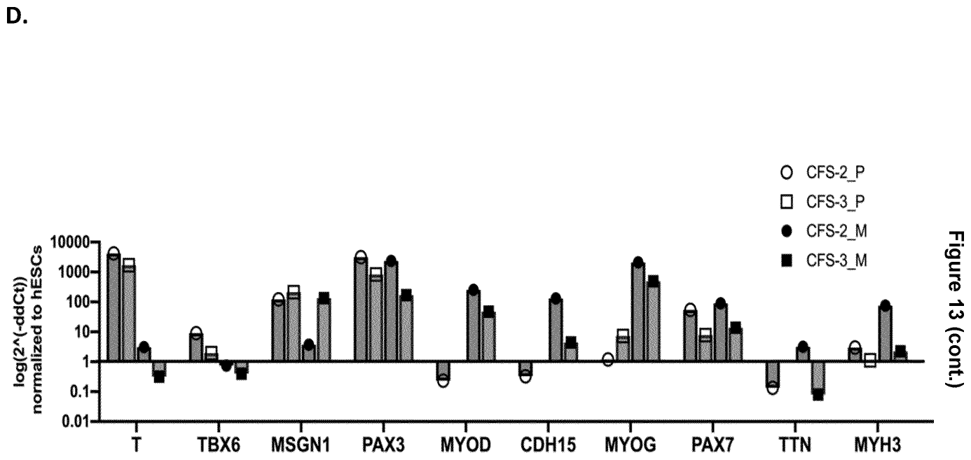

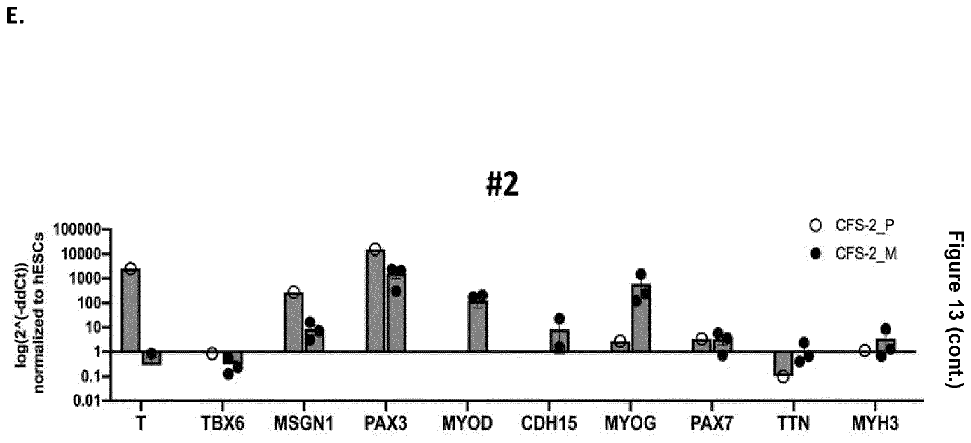

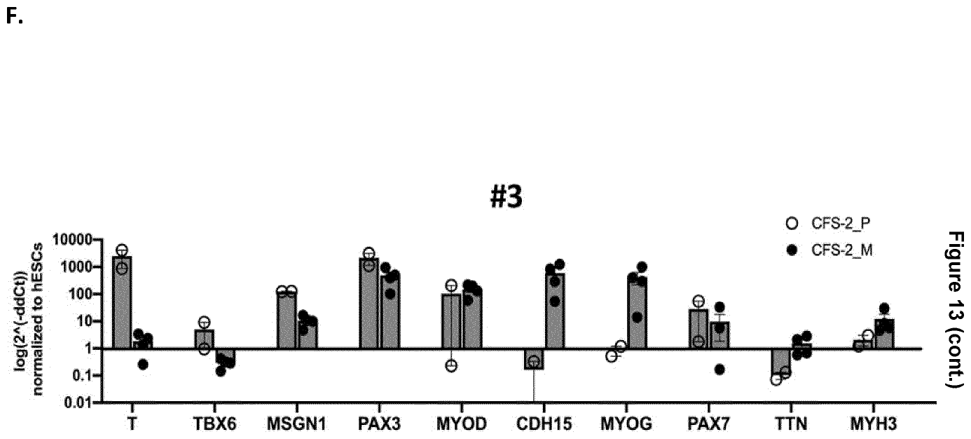

We investigated dermomyotomal progeny of axial stem cells by inducing skeletal muscle differentiation. We modified Choi et al., 2019 protocol and applied 4 differentiation modalities (FIG. 13A) to compare efficiency. CFS-derived cells were collected at DAY40 (FIG. 13B) however CS-derived cells showed strict neural morphology (FIG. 13C) and could not be maintained stably until DAY8. For analysis of differentiated progeny, we used stage-specific markers published in the literature. These markers are expressed during generation of skeletal muscle cells originating from paraxial mesoderm. TBXT is expressed in early mesodermal progenitors, following its downregulation, TBX6 and MSGN1 are upregulated to mark the pre-somitic mesoderm formation. Subsequently, PAX3 marks the appearance of dermomyotomal progenitors followed by MYOD. The latter is expressed in both myoblast progenitors and myoblasts cells. Expression of PAX7 helps to discriminate myoblasts and satellite-like cell state. Its co-expression with MYOD is characteristic of myoblasts, while PAX7 only is expressed in satellite-like cells. MYOG is used as an indicator of myocytes, while MYH3 and TTN are expressed in mature/fused muscle fibers. CDH15 is expressed in myoblasts, myocytes and mature muscle fibers. Based on gene expression analysis (FIG. 13D-G) and immunostaining results (FIG. 13H-J), CFS cells are able to generate myoblasts and myocytes like cells as high upregulation of MYOD and MYOG transcription factors as well as muscle specific MyHC and M-cadherin cytoskeletal proteins were detected.

We additionally analyzed skeletal muscle progeny of CFS lines by generating organoids. We applied similar protocol of 2D differentiation to 3D culture (FIG. 14A) and organoids were analyzed at DAY40 (FIG. 14B). Immunostaining of MyHC and ACTA1 (muscle specific actin) confirmed the skeletal muscle progeny (FIG. 14D). We also checked neural progeny by immunostaining of MNX1, ISL1 and OLIG2 which we detected their expression (FIG. 14F) therefore named the organoids as neuromuscular organoids.

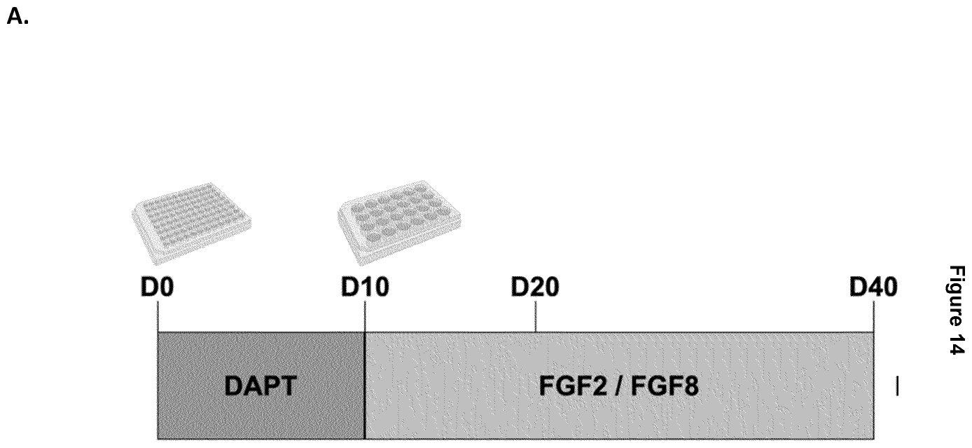

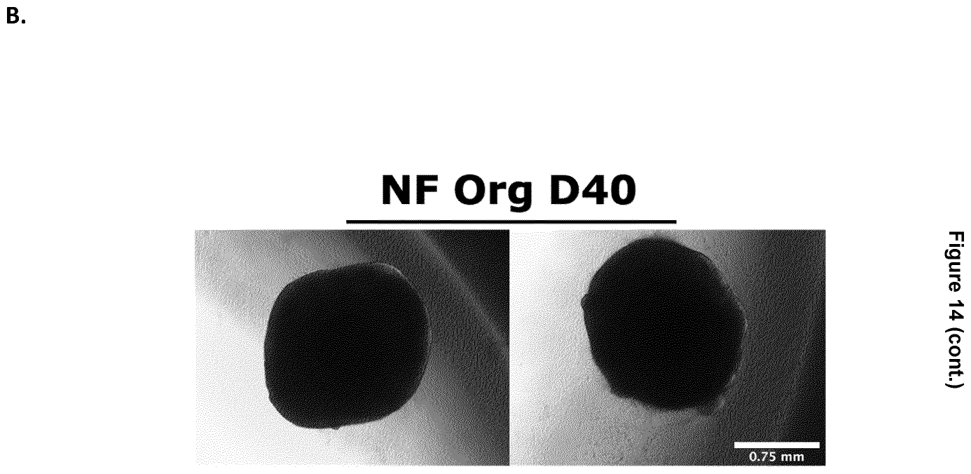

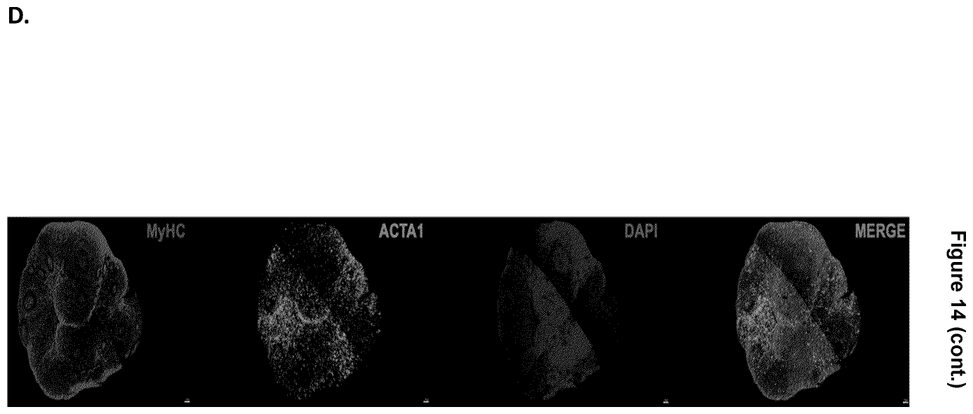

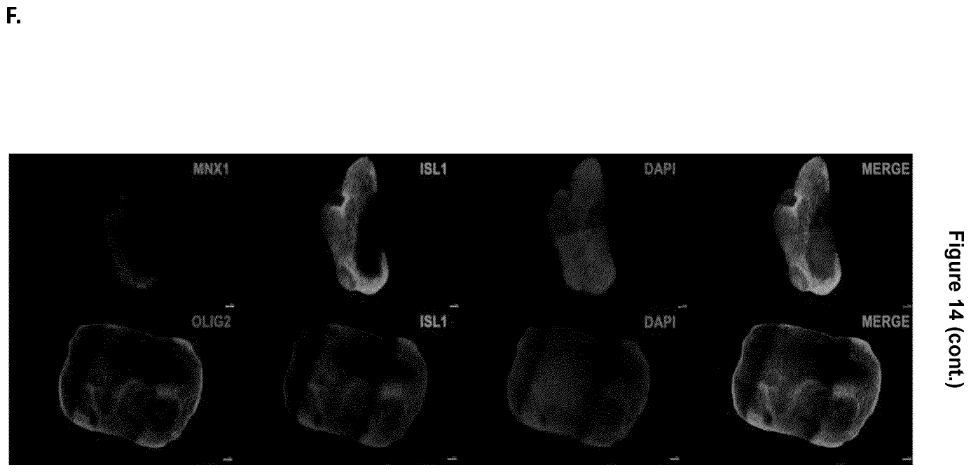

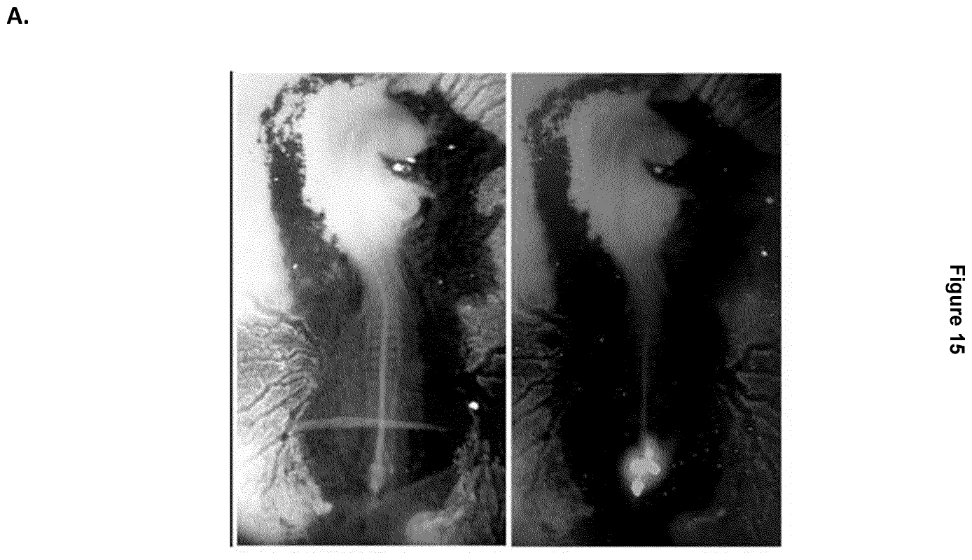

We finally investigated contribution of CFS line to neural tube and somites, which form dermomyotome and sclerotome subsequently, by injecting GFP(green fluorescent protein)-tagged CFS cells to chordoneural hinge of HH17 chick embryos (FIG. 15A). Embryo development was stopped at HH23-24 stage (FIG. 15B). GFP staining showed that CFS cells were located at neural tube and somites (FIG. 15C).

Discussion of Examples 4-5

SOX2 is known as a master regulator for self-renewal not only in pluripotent stem cells (Avilion et al., 2003, Boyer et al., 2005) but also in lineage specific stem cells (Graham et al., 2003, Favaro et al., 2009). T expression is a hallmark of early mesodermal progenitors (Showell et al., 2004, Tosic et al., 2019). Co-expression of these two critical transcription factors has been used to identify neuromesodermal progenitors (NMPs). NMPs have been identified in mouse, chick and human embryos. Due to our current scientific understanding of early development, it is possible by manipulating WNT & FGF signaling pathways to transiently generated NMPs in vitro. There have been no studies so far where long term in vitro maintenance of NMPs has been demonstrated. The commonly described NMP population (Henrique et al., 2015, Wymeersch et al., 2019) is transient and heterogeneous which poses a challenge for the usage of this developmental progenitor for disease modeling or the manufacturing of cell therapy products.

We herein introduce a novel type of regional stem cells namely axial stem cells (AxSCs) which could be derived from hESCs or hiPSCs by activation of Wnt/β-catenin and inhibition of TGFb signaling pathways with or without activation of FGF signaling. Based on the activation of the FGF signaling pathway, we have derived two AxSCs types which represent two developmental states and could be identified by expression of SOX2/T or SOX2/PAX6. We named SOX2/T expressing cells as CFS and SOX2/PAX6 expressing cells as CS. PAX6 expression has been shown to be a key determinant for neuroectodermal lineage formation as being one of the earliest identifiable markers (Zhang et al., 2010). By single cell sequencing analysis, we detected high PAX6 expression in CS cells in addition to SOX1 which is also a critical transcription factor to mark neuroectodermal cells (Zhang et al., 2010), while expression of NMP markers was not detected except for SOX2. As a result, we can conclude that the transcriptional profile of CS cells indicates neural bias. Furthermore, expression of DCX in a group of CS cells indicated that spontaneous differentiation within this population can be identified as immature neurons. On the other hand, CFS cells did not exhibit commitment to any lineages except for SOX2+ CDX2− cells. This subgroup of the CFS cells showed a clear upregulation of intermediate mesoderm markers and downregulation of neuromesodermal markers indicating spontaneous differentiation in this subgroup. As a result we can conclude that CDX2 expression is essential for maintenance of a less primed state in CFS cells. Notably, CDX2 downregulation coincides with SALL4 downregulation independent of SOX2 expression hinting that SALL4 can have a role in self-renewal of AxSCs. It has been shown in literature that SALL4 is an essential gene for self-renewal of human pluripotent stem cells (Yilmaz et al., 2018). The majority of the CFS cells were clustered in the SOX2+ CDX2+ cells, which represents an uncommitted state of CFS cells, as no lineage specific markers were detected. Based on these results it can be concluded that CFS cells represent a more heterogeneous, unbiased state of AxSCs. Therefore, we can describe the CS cells as primed AxSC state and the CFS cells as ground state of AxSCs based on their respective transcription profiles. We investigated the developmental potential of AxSCs for neural and dermomyotome lineages. First we set out to investigate the neural lineage by performing motor neuron differentiation from AxSCs. Based on literature we modulated pathways involved in spinal cord development and concomitant motor neuron specification (Stifani, 2014). Strikingly, neural morphology was observed in CS cells faster than CFS cells. The rapid change in morphology supports our hypothesis of a strong neural bias being present in the CS cells. Heterogeneity in differentiated cultures was indicative of a larger developmental propensity of the CFS cells in terms of neural progeny, as they are able to generate a mixture of sensory and motor neurons while CS cells have a higher propensity for generating homogenous differentiating cultures with either a sensory or a motor neuron bias. Additionally, we demonstrated that AxSCs are able to differentiate into mature neurons much faster than human pluripotent stem cells allowing AxSCs as a considerable advancement for cell therapy-based treatments. To confirm the dermomyotomal progeny of CFS cells we successfully differentiated AxSCs into skeletal muscle cells. Independent of the differentiation protocol we followed, there was no clear difference concerning the generation of myoblast progenitors or myoblast cells, but based on myocyte-like morphology we concluded that cAMP and vitamin C promote maturation of myoblasts. When we applied the same experimental conditions to CS cells, they displayed a clear neural morphology and did not generate stable cultures.

In conclusion, we have demonstrated the capacity of the CFS line to generate both neural and dermomyotomal progeny in vitro and its potential contribution to neural tube and somites in vivo. We have confirmed that CS cells are restricted to the neural lineage in terms of progeny and cannot give rise to any dermomyotome progeny. Thereby, based on the aforementioned conclusions, the hypothesized AxSCs hierarchy of ground/primed state of AxSCs is not only proven based on their transcriptional state but also on the resulting progeny from each AxSC population. Based on the development potential of our AxSCs, this work is the first study of its kind showing the in vitro isolation of a stem cell population representative of the in vivo NMPs. We believe the AxSCs could serve as a promising tool for developmental studies and manufacturing of potential cell therapy products for stem cell replacement therapy of peripheral nervous system degenerative diseases such as spinal muscular atrophy and amyotrophic lateral sclerosis.

### Example 6: the Axial Stem Cells (AxSCs) are not Neuromesodermal Progenitors (NMPs)

The axial stem cells (AxSCs) of the present invention are not neuromesodermal progenitors (NMPs), mainly because NMPs are transient, meaning that NMPs cannot be propagated, whereas the AxSCs are not transient cells and can be propagated and indefinitely renewed.

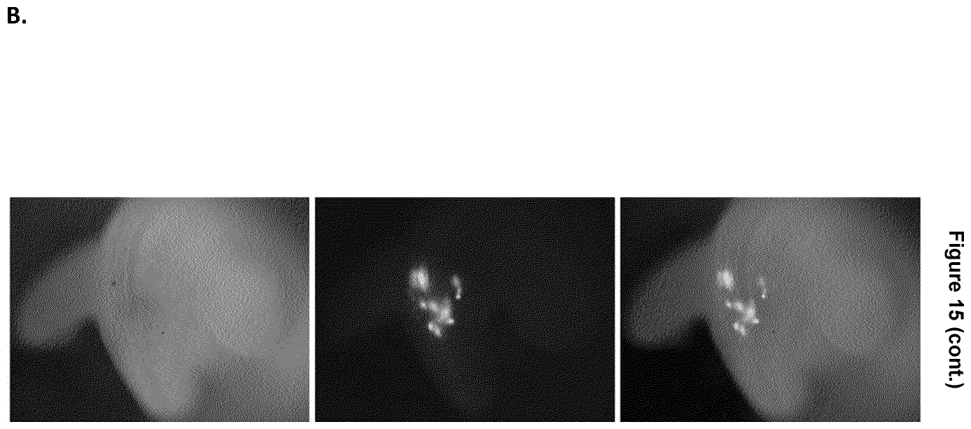

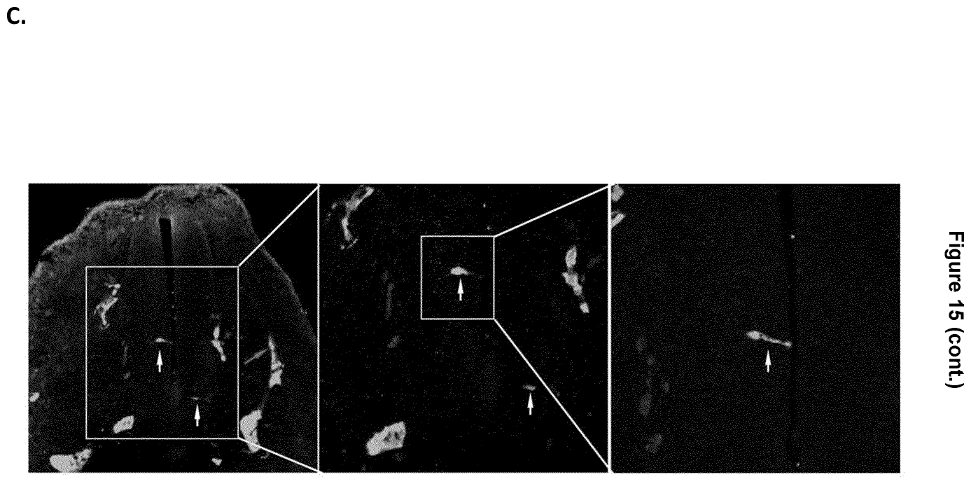

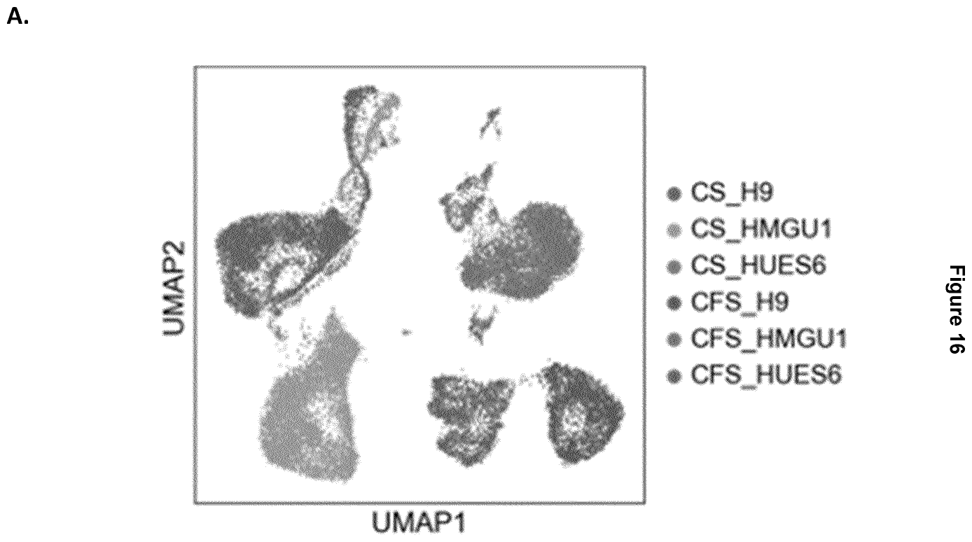

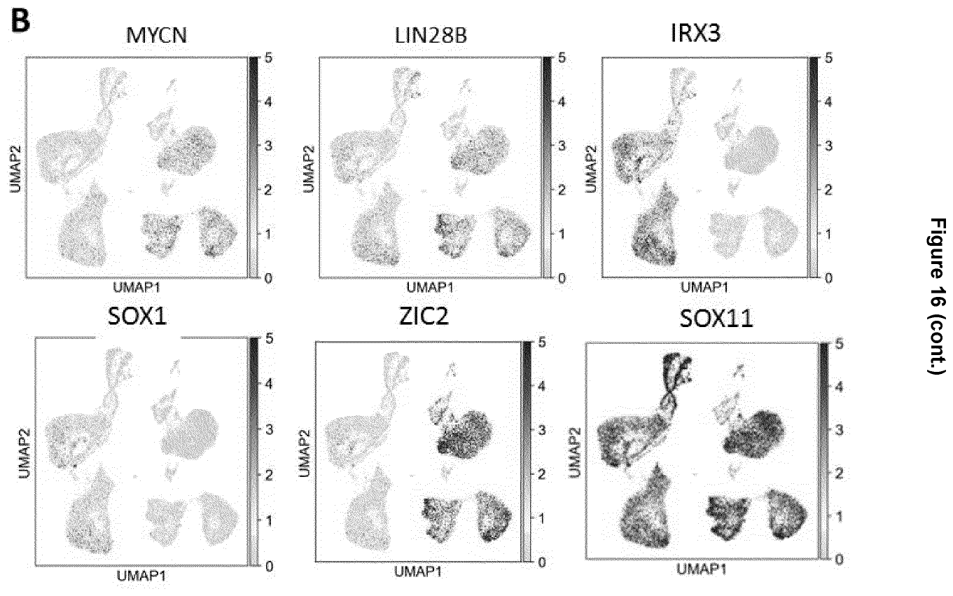

However, in order to further address the differences between AxSCs of the present invention and known NMPs we have looked at the “sternness” genes not expressed by NMPs, but which are expressed by the AxSCs. Self-renewing stem cells (e.g., as can be found in the embryo) have the tendency to express certain genes, and some of them are the so-called “Yanamaka factors”. Therefore, to address the differences between AxSCs and NMPs on the gene expression level we analyzed the genes that are essential for induced pluripotent stem (iPS) cells, embryonic stem cells (ESCs), collectively, pluripotent stem cells (PSCs). Our data shows that three of the Yamanaka genes (and that they are essential for hPSCs) are expressed together by AxSCs, namely, LIN28B (Protein lin-28 homolog B, e.g., having UniProtKB-Q6ZN17 or SEQ ID NO: 31), MYCN (N-myc proto-oncogene protein, e.g., having UniProtKB-P04198 or SEQ ID NO: 32) and SOX2 (Transcription factor SOX-2, e.g., having UniProtKB-P48431 or SEQ ID NO: 21). Notably, LIN28 and MYC have not been previously mentioned in the context of NMPs in vivo. Typically, the Yamanaka genes include LIN28A (Protein lin-28 homolog A, e.g., having UniProtKB-Q9H9Z2) and c-MYC (Myc proto-oncogene protein, e.g., having UniProtKB-P01106), whereas we have found the expression of LIN28B and MYCN in the AxSCs, but they are functionally very similar to LIN28A and c-MYC. Accordingly, this finding strongly supports the notion that the AxSCs of the present invention are very distinct from known NMPs (FIG. 16, A-B).

We have further looked at genes that are uniquely defining AxSCs, by comparing our RNA seq data to mouse NMPs in vivo. As can be seen from FIG. 16, we identified a panel of genes, namely, MYCN (N-myc proto-oncogene protein, e.g., having UniProtKB-P04198 or SEQ ID NO: 32), LIN28B (Protein lin-28 homolog B, e.g., having UniProtKB-Q6ZN17 or SEQ ID NO: 31), IRX3 (Iroquois-class homeodomain protein IRX-3, e.g., having UniProtKB-P78415 or SEQ ID NO: 34), SOX1 (Transcription factor SOX-1, e.g., having UniProtKB-O00570 or SEQ ID NO: 35), ZIC2 (Zinc finger protein ZIC 2, e.g., having UniProtKB-O95409 or SEQ ID NO: 33), SOX11 (Transcription factor SOX-11, e.g., having UniProtKB-P35716 or SEQ ID NO: 36) comprising genes that are prominently expressed by AxSCs (FIG. 16, AxSCs of the ground state (CFS on right) predominantly express MYCN, LIN28B, ZIC2 and SOX11; AxSCs of the primed state (CS on left) predominantly express MYCN, LIN28B, IRX3, SOX1, ZIC2 and SOX11), but NOT expressed by human NMPs derived from human ES cells (Verrier et al., 2018 Development) or Mouse NMPs from embryos (Gouti et al., Dev Cell. 2017).

Finally, we have shown that the AxSCs of the present invention can also be produced from iPSCs, hence, not involving the method of destruction of a human embryo. This further confirms our initial filing with respect to the human iPSC line HMGU #1.

### REFERENCES

1. Martin, G. R. (1981). Isolation of a pluripotent cell line from early mouse embryos cultured in medium conditioned by teratocarcinoma stem cells. Proc Natl Acad Sci USA 78, 7634-7638.
2. Evans, M. J., and Kaufman, M. H. (1981). Establishment in culture of pluripotential cells from mouse embryos. Nature 292, 154-156.
3. Tanaka, S., Kunath, T., Hadjantonakis, A. K., Nagy, A., and Rossant, J. (1998).

Promotion of trophoblast stem cell proliferation by FGF4. Science 282, 2072-2075.
4. Niakan, K. K., Schrode, N., Cho, L. T., and Hadjantonakis, A. K. (2013). Derivation of extraembryonic endoderm stem (XEN) cells from mouse embryos and embryonic stem cells. Nat Protoc 8, 1028-1041.
5. Smith, A. (2017). Formative pluripotency: the executive phase in a developmental continuum. Development 144, 365-373.
6. Nichols, J., and Smith, A. (2009). Naive and primed pluripotent states. Cell Stem Cell 4, 487-492.
7. Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., and Vallier, L. (2007). Derivation of pluripotent epiblast stem cells from mammalian embryos. Nature 448, 191-195.
8. Tesar, P. J. (2005). Derivation of germ-line-competent embryonic stem cell lines from preblastocyst mouse embryos. Proc Natl Acad Sci USA 102, 8239-8244.
9. Thomson, J. A., ltskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
10. Gafni, O., Weinberger, L., Mansour, A. A., Manor, Y. S., Chomsky, E., Ben-Yosef, D., Kalma, Y., Viukov, S., Maza, I., Zviran, A., Rais, Y., Shipony, Z., Mukamel, Z., Krupalnik, V., Zerbib, M., Geula, S., Caspi, I., Schneir, D., Shwartz, T., Gilad, S., Amann-Zalcenstein, D., Benjamin, S., Amit, I., Tanay, A., Massarwa, R., Novershtern, N., and Hanna, J. H. (2013). Derivation of novel human ground state naive pluripotent stem cells. Nature 504, 282-286.
11. Guo, G., von Meyenn, F., Santos, F., Chen, Y., Reik, W., Bertone, P., Smith, A., and Nichols, J. (2016). Naive Pluripotent Stem Cells Derived Directly from Isolated Cells of the Human Inner Cell Mass. Stem Cell Reports 6, 437-446.
12. Takashima, Y., Guo, G., Loos, R., Nichols, J., Ficz, G., Krueger, F., Oxley, D., Santos, F., Clarke, J., Mansfield, W., Reik, W., Bertone, P., and Smith, A. (2014). Resetting transcription factor control circuitry toward ground-state pluripotency in human. Cell 158, 1254-1269.
13. Theunissen, T. W., Powell, B. E., Wang, H., Mitalipova, M., Faddah, D. A., Reddy, J., Fan, Z. P., Maetzel, D., Ganz, K., Shi, L., Lungjangwa, T., Imsoonthornruksa, S., Stelzer, Y., Rangarajan, S., D'Alessio, A., Zhang, J., Gao, Q., Dawlaty, M. M., Young, R. A., Gray, N. S., and Jaenisch, R. (2014). Systematic Identification of Culture Conditions for Induction and Maintenance of Naive Human Pluripotency. Cell Stem Cell 15, 524-526.
14. Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
15. Takahashi, K., and Yamanaka, S. (2006). Induction of pluripotent stem cells from mouse embryonic and adult fibroblast cultures by defined factors. Cell 126, 663-676.
16. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, II, and Thomson, J. A. (2007). Induced pluripotent stem cell lines derived from human somatic cells. Science 318, 1917-1920.
17. Liu, H., Zhu, F., Yong, J., Zhang, P., Hou, P., Li, H., Jiang, W., Cai, J., Liu, M., Cui, K., Qu, X., Xiang, T., Lu, D., Chi, X., Gao, G., Ji, W., Ding, M., and Deng, H. (2008). Generation of induced pluripotent stem cells from adult rhesus monkey fibroblasts. Cell Stem Cell 3, 587-590.
18. Benazeraf, B., and Pourquie, O. (2013). Formation and segmentation of the vertebrate body axis. Annu Rev Cell Dev Biol 29, 1-26.
19. Henrique, D., Abranches, E., Verrier, L., and Storey, K. G. (2015). Neuromesodermal progenitors and the making of the spinal cord. Development 142, 2864-2875.
20. Cambray, N., and Wilson, V. (2002). Axial progenitors with extensive potency are localised to the mouse chordoneural hinge. Development 129, 4855-4866.
21. Cambray, N., and Wilson, V. (2007). Two distinct sources for a population of maturing axial progenitors. Development 134, 2829-2840.
22. Garriock, R. J., Chalamalasetty, R. B., Kennedy, M. W., Canizales, L. C., Lewandoski, M., and Yamaguchi, T. P. (2015). Lineage tracing of neuromesodermal progenitors reveals novel Wnt-dependent roles in trunk progenitor cell maintenance and differentiation. Development 142, 1628-1638.
23. Attardi, A., Fulton, T., Florescu, M., Shah, G., Muresan, L., Lenz, M. O., Lancaster, C., Huisken, J., van Oudenaarden, A., and Steventon, B. (2018). Neuromesodermal progenitors are a conserved source of spinal cord with divergent growth dynamics. Development 145.
24. Tzouanacou, E., Wegener, A., Wymeersch, F. J., Wilson, V., and Nicolas, J. F. (2009). Redefining the progression of lineage segregations during mammalian embryogenesis by clonal analysis. Dev Cell 17, 365-376.
25. McGrew, M. J., Sherman, A., Lillico, S. G., Ellard, F. M., Radcliffe, P. A., Gilhooley, H. J., Mitrophanous, K. A., Cambray, N., Wilson, V., and Sang, H. (2008). Localised axial progenitor cell populations in the avian tail bud are not committed to a posterior Hox identity. Development 135, 2289-2299.
26. Delfino-Machin, M., Lunn, J. S., Breitkreuz, D. N., Akai, J., and Storey, K. G. (2005). Specification and maintenance of the spinal cord stem zone. Development 132, 4273-4283.
27. Tsakiridis, A., Huang, Y., Blin, G., Skylaki, S., Wymeersch, F., Osorno, R., Economou, C., Karagianni, E., Zhao, S., Lowell, S., and Wilson, V. (2014). Distinct Wnt-driven primitive streak-like populations reflect in vivo lineage precursors. Development 141, 1209-1221.
28. Gouti, M., Tsakiridis, A., Wymeersch, F. J., Huang, Y., Kleinjung, J., Wilson, V., and Briscoe, J. (2014). In vitro generation of neuromesodermal progenitors reveals distinct roles for wnt signalling in the specification of spinal cord and paraxial mesoderm identity. PLoS Biol 12, e1001937.
29. Koch, F., Scholze, M., Wittier, L., Schifferl, D., Sudheer, S., Grote, P., Timmermann, B., Macura, K., and Herrmann, B. G. (2017). Antagonistic Activities of Sox2 and Brachyury Control the Fate Choice of Neuro-Mesodermal Progenitors. Dev Cell 42, 514-526 e517.
30. Wilson, V., and Beddington, R. S. (1996). Cell fate and morphogenetic movement in the late mouse primitive streak. Mech Dev 55, 79-89.
31. Kinder, S. J., Tsang, T. E., Quinlan, G. A., Hadjantonakis, A. K., Nagy, A., and Tam, P. P. (1999). The orderly allocation of mesodermal cells to the extraembryonic structures and the anteroposterior axis during gastrulation of the mouse embryo. Development 126, 4691-4701.
32. Smith, J. L., Gesteland, K. M., and Schoenwolf, G. C. (1994). Prospective fate map of the mouse primitive streak at 7.5 days of gestation. Dev Dyn 201, 279-289.
33. Taguchi, A., Kaku, Y., Ohmori, T., Sharmin, S., Ogawa, M., Sasaki, H., and Nishinakamura, R. (2014). Redefining the in vivo origin of metanephric nephron progenitors enables generation of complex kidney structures from pluripotent stem cells. Cell Stem Cell 14, 53-67.
34. Wymeersch, F. J., Skylaki, S., Huang, Y., Watson, J. A., Economou, C., Marek-Johnston, C., Tomlinson, S. R., and Wilson, V. (2019). Transcriptionally dynamic progenitor populations organised around a stable niche drive axial patterning. Development 146.
35. Turner, D. A., Hayward, P. C., Baillie-Johnson, P., Rue, P., Broome, R., Faunes, F., and Martinez Arias, A. (2014). Wnt/beta-catenin and FGF signalling direct the specification and maintenance of a neuromesodermal axial progenitor in ensembles of mouse embryonic stem cells. Development 141, 4243-4253.
36. Lippmann, E. S., Williams, C. E., Ruhl, D. A., Estevez-Silva, M. C., Chapman, E. R., Coon, J. J., and Ashton, R. S. (2015). Deterministic HOX patterning in human pluripotent stem cell-derived neuroectoderm. Stem Cell Reports 4, 632-644.
37. Tsakiridis, A., and Wilson, V. (2015). Assessing the bipotency of in vitro-derived neuromesodermal progenitors. F1000Res 4, 100.
38. Gouti, M., Delile, J., Stamataki, D., Wymeersch, F. J., Huang, Y., Kleinjung, J., Wilson, V., and Briscoe, J. (2017). A Gene Regulatory Network Balances Neural and Mesoderm Specification during Vertebrate Trunk Development. Dev Cell 41, 243-261 e247.
39. Denham, M., Hasegawa, K., Menheniott, T., Rollo, B., Zhang, D., Hough, S., Alshawaf, A., Febbraro, F., lghaniyan, S., Leung, J., Elliott, D. A., Newgreen, D. F., Pera, M. F., and Dottori, M. (2015). Multipotent caudal neural progenitors derived from human pluripotent stem cells that give rise to lineages of the central and peripheral nervous system. Stem Cells 33, 1759-1770.
40. Verrier, L., Davidson, L., Gierlinski, M., Dady, A., and Storey, K. G. (2018). Neural differentiation, selection and transcriptomic profiling of human neuromesodermal progenitor-like cells in vitro. Development 145.
41. Edri, S., Hayward, P., Baillie-Johnson, P., Steventon, B., and Martinez Arias, A. (2018). An Epiblast Stem Cell derived multipotent progenitor population for axial extension. Bioarxiv.
42. Arnold, S. J., Stappert, J., Bauer, A., Kispert, A., Herrmann, B. G., and Kemler, R. (2000). Brachyury is a target gene of the Wnt/beta-catenin signaling pathway. Mech Dev 91, 249-258.
43. Lian, X., Hsiao, C., Wilson, G., Zhu, K., Hazeltine, L. B., Azarin, S. M., Raval, K. K., Zhang, J., Kamp, T. J., and Palecek, S. P. (2012). Robust cardiomyocyte differentiation from human pluripotent stem cells via temporal modulation of canonical Wnt signaling. Proc Natl Acad Sci U S A 109, E1848-1857.
44. Kitchens, D. L., Snyder, E. Y., and Gottlieb, D. I. (1994). FGF and EGF are mitogens for immortalized neural progenitors. J Neurobiol 25, 797-807.
45. Wilson, R., Vogelsang, E., and Leptin, M. (2005). FGF signalling and the mechanism of mesoderm spreading in Drosophila embryos. Development 132, 491-501.
46. Komori, T. (2010). Regulation of osteoblast differentiation by Runx2. Adv Exp Med Biol 658, 43-49.
47. Boyer, L. A., Lee, T. I., Cole, M. F., Johnstone, S. E., Levine, S. S., Zucker, J. P., Guenther, M. G., Kumar, R. M., Murray, H. L., Jenner, R. G., Gifford, D. K., Melton, D. A., Jaenisch, R., and Young, R. A. (2005). Core transcriptional regulatory circuitry in human embryonic stem cells. Cell 122, 947-956.

48. Avilion, A. A., Nicolis, S. K., Pevny, L. H., Perez, L., Vivian, N., and Lovell-Badge, R. (2003). Multipotent cell lineages in early mouse development depend on SOX2 function. Genes Dev 17, 126-140.
49. Graham, V., Khudyakov, J., Ellis, P., and Pevny, L. (2003). SOX2 functions to maintain neural progenitor identity. Neuron 39, 749-765.
50. Pevny, L. H., and Nicolis, S. K. (2010). Sox2 roles in neural stem cells. Int J Biochem Cell Biol 42, 421-424.
51. Cimadamore, F., Fishwick, K., Giusto, E., Gnedeva, K., Cattarossi, G., Miller, A., Pluchino, S., Brill, L. M., Bronner-Fraser, M., and Terskikh, A. V. (2011). Human ESC-derived neural crest model reveals a key role for SOX2 in sensory neurogenesis. Cell Stem Cell 8, 538-551.
52. Martin, B. L., and Kimelman, D. (2010). Brachyury establishes the embryonic mesodermal progenitor niche. Genes Dev 24, 2778-2783.
53. Zhang, X., Huang, C. T., Chen, J., Pankratz, M. T., Xi, J., Li, J., Yang, Y., Lavaute, T. M., Li, X. J., Ayala, M., Bondarenko, G. I., Du, Z. W., Jin, Y., Golos, T. G., and Zhang, S. C. (2010). Pax6 is a human neuroectoderm cell fate determinant. Cell Stem Cell 7, 90-100.
54. Greber, B., Coulon, P., Zhang, M., Moritz, S., Frank, S., Muller-Molina, A. J., Arauzo-Bravo, M. J., Han, D. W., Pape, H. C., and Scholer, H. R. (2011). FGF signalling inhibits neural induction in human embryonic stem cells. EMBO J 30, 4874-4884.
55. Yao, Y., Li, W., Wu, J., Germann, U. A., Su, M. S., Kuida, K., and Boucher, D. M. (2003). Extracellular signal-regulated kinase 2 is necessary for mesoderm differentiation. Proc Natl Acad Sci USA 100, 12759-12764.
56. Takemoto, T., Uchikawa, M., Kamachi, Y., and Kondoh, H. (2006). Convergence of Wnt and FGF signals in the genesis of posterior neural plate through activation of the Sox2 enhancer N-1. Development 133, 297-306.
57. Kondoh, H., and Takemoto, T. (2012). Axial stem cells deriving both posterior neural and mesodermal tissues during gastrulation. Curr Opin Genet Dev 22, 374-380.
58. Young, T., Rowland, J. E., van de Ven, C., Bialecka, M., Novoa, A., Carapuco, M., van Nes, J., de Graaff, W., Duluc, I., Freund, J. N., Beck, F., Mallo, M., and Deschamps, J. (2009). Cdx and Hox genes differentially regulate posterior axial growth in mammalian embryos. Dev Cell 17, 516-526.
59. Amin, S., Neijts, R., Simmini, S., van Rooijen, C., Tan, S. C., Kester, L., van Oudenaarden, A., Creyghton, M. P., and Deschamps, J. (2016). Cdx and T Brachyury Co-activate Growth Signaling in the Embryonic Axial Progenitor Niche. Cell Rep 17,
60. Kunze, C., Borner, K., Kienle, E., Orschmann, T., Rusha, E., Schneider, M., Radivojkov-Blagojevic, M., Drukker, M., Desbordes, S., Grimm, D., and Brack-Werner, R. (2018). Synthetic AAV/CRISPR vectors for blocking HIV-1 expression in persistently infected astrocytes. Glia 66, 413-427.
61. Livak, K. J., and Schmittgen, T. D. (2001). Analysis of relative gene expression data using real-time quantitative PCR and the 2(-Delta Delta C(T)) Method. Methods 25, 402-408.
62. Wickham, H. (2009). ggplot2: Elegant Graphics for Data Analysis (New York: Springer-Verlag New York).
63. R Core Team (2018). R: A language and environment for statistical computing (Vienna, Austria: R Foundation for Statistical Computing).
64. Bray, N. L., Pimentel, H., Melsted, P., and Pachter, L. (2016). Near-optimal probabilistic RNA-seq quantification. Nat Biotechnol 34, 525-527.
65. Soneson, C., Love, M. I., and Robinson, M. D. (2015). Differential analyses for RNA-seq: transcript-level estimates improve gene-level inferences. F1000Res 4, 1521.
66. Love, M. I., Huber, W., and Anders, S. (2014). Moderated estimation of fold change and dispersion for RNA-seq data with DESeq2. Genome Biol 15, 550.
67. Afgan, E., Baker, D., Batut, B., van den Beek, M., Bouvier, D., Cech, M., Chilton, J., Clements, D., Coraor, N., Gruning, B. A., Guerler, A., Hillman-Jackson, J., Hiltemann, S., Jalili, V., Rasche, H., Soranzo, N., Goecks, J., Taylor, J., Nekrutenko, A., and Blankenberg, D. (2018). The Galaxy platform for accessible, reproducible and collaborative biomedical analyses: 2018 update. Nucleic Acids Res 46, W537-W544.
68. Bolger, A. M., Lohse, M., and Usadel, B. (2014). Trimmomatic: a flexible trimmer for Illumina sequence data. Bioinformatics 30, 2114-2120.
69. Kim, D., Langmead, B., and Salzberg, S. L. (2015). HISAT: a fast spliced aligner with low memory requirements. Nat Methods 12, 357-360.
70. Liao, Y., Smyth, G. K., and Shi, W. (2014). featureCounts: an efficient general purpose program for assigning sequence reads to genomic features. Bioinformatics 30, 923-930.
71. Alexa, A., and Rahnenfuhrer, J. (2018). topGO: Enrichment Analysis for Gene Ontology.
72. Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., and Ma'ayan, A. (2016). Enrichr: a comprehensive gene set enrichment analysis web server 2016 update. Nucleic Acids Res 44, W90-97.
73. Avilion A A, Nicolis S K, Pevny L H, Perez L, Vivian N, Lovell-Badge R. Multipotent cell lineages in early mouse development depend on SOX2 function. Genes Dev. 2003 Jan. 1; 17(1):126-40. doi: 10.1101/gad.224503. PMID: 12514105; PMCID: PMC195970.
74. Boyer L A, Lee T I, Cole M F, Johnstone S E, Levine S S, Zucker J P, Guenther M G, Kumar R M, Murray H L, Jenner R G, Gifford D K, Melton D A, Jaenisch R, Young R A. Core transcriptional regulatory circuitry in human embryonic stem cells. Cell. 2005 Sep. 23; 122(6):947-56. doi: 10.1016/j.ce11.2005.08.020. PMID: 16153702; PMCID: PMC3006442.
75. Favaro R, Valotta M, Ferri A L, Latorre E, Mariani J, Giachino C, Lancini C, Tosetti V, Ottolenghi S, Taylor V, Nicolis S K. Hippocampal development and neural stem cell maintenance require Sox2-dependent regulation of Shh. Nat Neurosci. 2009 October; 12(10):1248-56. doi: 10.1038/nn.2397. Epub 2009 Sep. 6. PMID: 19734891.
76. Graham V, Khudyakov J, Ellis P, Pevny L. SOX2 functions to maintain neural progenitor identity. Neuron. 2003 Aug. 28; 39(5):749-65. doi: 10.1016/s0896-6273(03)00497-5. PMID: 12948443.
77. Henrique D, Abranches E, Verrier L, Storey K G. Neuromesodermal progenitors and the making of the spinal cord. Development. 2015 Sep. 1; 142(17):2864-75. doi: 10.1242/dev.119768. PMID: 26329597; PMCID: PMC4958456.
78. Showell C, Binder O, Conlon F L. T-box genes in early embryogenesis. Dev Dyn. 2004 January; 229(1):201-18. doi: 10.1002/dvdy.10480. PMID: 14699590; PMCID: PMC1635809.
79. Stifani N. Motor neurons and the generation of spinal motor neuron diversity. Front Cell Neurosci. 2014 Oct. 9; 8:293. doi: 10.3389/fnce1.2014.00293. PMID: 25346659; PMCID: PMC4191298.
80. Tosic J, Kim G J, Pavlovic M, Schröder C M, Mersiowsky S L, Barg M, Hofherr A,

Probst S, Köttgen M, Hein L, Arnold S J. Eomes and Brachyury control pluripotency exit and germlayer segregation by changing the chromatin state. Nat Cell Biol. 2019 December; 21(12):1518-1531. doi: 10.1038/s41556-019-0423-1. Epub 2019 Dec. 2. Erratum in: Nat Cell Biol. 2020 January; 22(1):135. PMID: 31792383.
81. Wymeersch F J, Skylaki S, Huang Y, Watson J A, Economou C, Marek-Johnston C, Tomlinson S R, Wilson V. Transcriptionally dynamic progenitor populations organised around a stable niche drive axial patterning. Development. 2019 Jan. 2; 146(1):dev168161. doi: 10.1242/dev.168161. PMID: 30559277; PMCID: PMC6340148.
82. Zhang X, Huang C T, Chen J, Pankratz M T, Xi J, Li J, Yang Y, Lavaute T M, Li X J, Ayala M, Bondarenko G I, Du Z W, Jin Y, Gobs T G, Zhang S C. Pax6 is a human neuroectoderm cell fate determinant. Cell Stem Cell. 2010 Jul. 2; 7(1):90-100. doi: 10.1016/j.stem.2010.04.017. PMID: 20621053; PMCID: PMC2904346.
83. Yilmaz A, Peretz M, Aharony A, Sagi I, Benvenisty N. Defining essential genes for human pluripotent stem cells by CRISPR-Cas9 screening in haploid cells. Nat Cell Biol. 2018 May; 20(5):610-619. doi: 10.1038/s41556-018-0088-1. Epub 2018 Apr 16. PMID: 29662178.

1. A method for producing axial stem cells (AxSCs), said method comprising:
a) providing pluripotent stem cells, embryonic or induced pluripotent stem cells;
b) activating the Wnt/β-catenin signaling pathway in said pluripotent stem cells, embryonic or induced pluripotent stem cells;
c) passaging the cells derived from the step (b) under the condition of continuous activation of the Wnt/β-catenin signaling pathway in said cells during said passaging, wherein the cells derived from the step (c) endogenously express transcription factor SOX-2;
d) optionally, said continuous activating the Wnt/β-catenin signaling pathway from the step (c) is carried out in the presence of: Fibroblast growth factor 2, TGF-β inhibitor, or both Fibroblast growth factor 2 and a TGF-β inhibitor.

2. The method for producing axial stem cells according to claim 1, wherein said activation of the Wnt/β-catenin signaling pathway is carried out by the means of using an inhibitor of GSK3b protein.

3. The method for producing axial stem cells according to claim 1 comprising step (d), wherein
a) said continuous activating the Wnt/β-catenin signaling from the step (c) is carried out with CHIR99021 inhibitor at a concentration of about 5 μM in the presence of: Fibroblast growth factor 2 and a TGF-β inhibitor; wherein said TGF-β inhibitor is SB-431542, wherein said SB-431542 inhibitor is used at a concentration of about 10 μM, wherein said Fibroblast growth factor 2 is used at a concentration from about 20 to about 100 ng/ml; wherein the derived cells endogenously express transcription factor SOX-2, T-box transcription factor T and Homeobox protein MIXL1 and do not endogenously express paired box protein Pax-6; and wherein optionally, said cells further endogenously express Homeobox protein CDX-2; or
b) said continuous activating the Wnt/β-catenin signaling from the step (c) is carried out with CHIR99021 inhibitor at a concentration of about 5 μM in the presence of a TGF-β inhibitor, wherein said TGF-β inhibitor is SB-431542, wherein said SB-431542 inhibitor is used at a concentration of about 10 μM; wherein the derived cells endogenously express transcription factor SOX-2 and paired box protein Pax-6 and do not endogenously express T-box transcription factor T, Homeobox protein MIXL1 and Homeobox protein CDX-2; or
c) said continuous activating the Wnt/β-catenin signaling from the step (c) is carried out with CHIR99021 inhibitor at a concentration of about 7.5 μM in the presence of a TGF-β inhibitor, wherein said TGF-β inhibitor is SB-431542, wherein said SB-431542 inhibitor is used at a concentration of about 10 μM, wherein the derived cells endogenously express transcription factor SOX-2, T-box transcription factor T, Homeobox protein MIXL1 and paired box protein Pax-6; wherein optionally, said cells further endogenously express Homeobox protein CDX-2.

4. The method for producing axial stem cells according to claim 1, wherein said axial stem cells have one or more of the following characteristics:
i) expressing transcription factor SOX-2, comprising UniProtKB-P48431 or SEQ ID NO: 21;
ii) substantially not expressing OCT4 transcription factor, comprising UniProtKB-Q01860 or SEQ ID NO: 22;
iii) substantially not expressing homeobox protein NANOG, comprising UniProtKB-Q9H9S0 or SEQ ID NO: 23;
iv) are not pluripotent;
v) are a region-specific multipotent stem cells;
vi) are obtainable from a pluripotent stem cell, embryonic or induced pluripotent stem cell;
vii) are not capable of differentiating into cell types of all tissues of the embryo;
viii) are only capable of differentiating in cell types emerging from the region of the central body axis during embryonic development, selected from:
sclerotome, dermomyotome and peripheral neurons;
ix) are not capable to form teratomas;
x) are capable of mimicking the characteristics of the precursors that give rise to the axial region, selected from: motor neurons, peripheral neurons, peripheral nervous system neurons sensory neurons, bone, cartilage, tendon, ligament and skeletal muscle cells;
xi) are not transient cells;
xii) are capable of differentiating into a motor neuron, peripheral neuron, muscle, cartilage or bone progenitor;
xiii) are indefinitely renewing stem cells;
xiv) are capable to grow as clones;
xv) are not neuromesodermal progenitors (NMps) and/or
xvi) is not induced neural stem cells (iNSCs).

5. The method for producing axial stem cells according claim 1, wherein said axial stem cells are not neuromesodermal progenitors (NMps), wherein said axial stem cells expressing one or more of the following proteins:
i) N-myc proto-oncogene protein (MYCN), comprising UniProtKB-P04198 or SEQ ID NO: 32;
ii) Protein lin-28 homolog B (LIN28B), comprising UniProtKB-Q6ZN17 or SEQ ID NO: 31;
iii) Iroquois-class homeodomain protein IRX-3 (IRX3), comprising UniProtKB-P78415 or SEQ ID NO: 34;
iv) Transcription factor SOX-1 (SOX1), comprising UniProtKB-O00570 or SEQ ID NO: 35;
v) Zinc finger protein ZIC 2 (ZIC2), comprising UniProtKB-O95409 or SEQ ID NO: 33;
vi) Transcription factor SOX-11 (SOX11), comprising UniProtKB-P35716 or SEQ ID NO: 36
wherein optionally, said AxSCs are ground state AxSCs expressing MYCN, LIN28B, ZIC2 and SOX11; or
said AxSCs are primed state AxSCs expressing MYCN, LIN28B, IRX3, SOX1, ZIC2 and SOX11.

6. The method for producing axial stem cells according to claim 1, wherein said axial stem cells are capable of indefinitely renewing and differentiating into:
i) ground axial stem cells, wherein said ground axial stem cells are indefinitely renewing ground axial stem cells; wherein said ground axial stem cell, expressing:
a) transcription factor SOX-2, comprising UniProtKB-P48431 or SEQ ID NO: 21;
b) T-box transcription factor T, comprising UniProtKB-O15178 or SEQ ID NO: 24;
c) homeobox protein CDX-2, comprising UniProtKB-Q99626 or SEQ ID NO: 25; and
d) homeobox protein MIXL1, comprising UniProtKB-Q9H2W2 or SEQ ID NO: 26;
ii) primed state axial stem cells, wherein said primed state axial stem cells are indefinitely renewing primed state axial stem cells; wherein said primed axial stem cell, expressing:
e) transcription factor SOX-2, comprising UniProtKB-P48431 or SEQ ID NO: 21 and
f) paired box protein PAX-6, comprising UniProtKB-P26367 or SEQ ID NO: 27.

7. The method for producing axial stem cells according to claim 1, wherein said axial stem cells are human axial stem cells.

8. (canceled)

9. An isolated axial stem cell (AxSC) produced by the method according to claim 1 or wherein said axial stem cell expresses the transcription factor SOX-2, substantially not expressing OCT4 transcription factor and substantially not expressing homeobox protein NANOG, wherein said AxSC is not pluripotent, wherein said AxSC further has one or more of the following characteristics:
i) is a region-specific multipotent stem cell;
ii) is obtainable from a pluripotent stem cell, embryonic or induced pluripotent stem cell;
iii) is not capable of differentiating into cell types of all tissues of the embryo;
iv) is only capable of differentiating in cell types emerging from the region of the central body axis during embryonic development, selected from: sclerotome, dermomyotome and peripheral neurons;
v) is not capable to form teratomas;
vi) is capable of mimicking the characteristics of the precursors that give rise to the axial region, selected from: motor neurons, peripheral neurons, peripheral nervous system neurons sensory neurons, bone, cartilage, tendon, ligament and skeletal muscle cells;
vii) is not a transient cell;
viii) is capable of differentiating into a motor neuron, peripheral neuron, muscle, cartilage or bone progenitor;
ix) is an indefinitely renewing stem cell;
x) is capable to grow as clones;
xi) is not a neuromesodermal progenitor (NMp);
xii) is not an induced neural stem cell (iNSC).

10. The axial stem cell according to claim 9, wherein said axial stem cell is not a neuromesodermal progenitor (NMp), wherein said axial stem cell expressing one or more of the following proteins:
i) N-myc proto-oncogene protein (MYCN), comprising UniProtKB-P04198 or SEQ ID NO: 32;
ii) Protein lin-28 homolog B (LIN28B), comprising UniProtKB-Q6ZN17 or SEQ ID NO: 31;
iii) Iroquois-class homeodomain protein IRX-3 (IRX3), comprising UniProtKB-P78415 or SEQ ID NO: 34;
iv) Transcription factor SOX-1 (SOX1), comprising UniProtKB-O00570 or SEQ ID NO: 35;
v) Zinc finger protein ZIC 2 (ZIC2), comprising UniProtKB-O95409 or SEQ ID NO: 33;
vi) Transcription factor SOX-11 (SOX11), comprising UniProtKB-P35716 or SEQ ID NO: 36;
wherein optionally, said AxSC is a ground state AxSC expressing MYCN, LIN28B, ZIC2 and SOX11;
or said AxSC is a primed state AxSC expressing MYCN, LIN28B, IRX3, SOX1, ZIC2 and SOX11.

11. The axial stem cell according to claim 9, wherein said axial stem cell is capable of indefinitely renewing itself and differentiating into:
i) a ground axial stem cell, wherein said ground axial stem cell, expressing:
a) transcription factor SOX-2, comprising UniProtKB-P48431 or SEQ ID NO: 21;
b) T-box transcription factor T, comprising UniProtKB-O15178 or SEQ ID NO: 24;
c) homeobox protein CDX-2, comprising UniProtKB-Q99626 or SEQ ID NO: 25; and
d) homeobox protein MIXL1, comprising UniProtKB-Q9H2W2 or SEQ ID NO: 26;
ii) primed state axial stem cell, wherein said primed axial stem cell, expressing:
e) transcription factor SOX-2, comprising UniProtKB-P48431 or SEQ ID NO: 21 and
f) paired box protein PAX-6, comprising UniProtKB-P26367 or SEQ ID NO: 27.

12. The axial stem cell according to claim 9, wherein said axial stem cell is a human axial stem cell.

13. (canceled)

14. A composition, preparation or kit comprising the axial stem cell according to claim 9.

15. (canceled)

16. (canceled)

17. A method of diagnosing, treating, ameliorating, or the prophylaxis of a neurodegenerative disease, a bone or cartilage disorder, a muscle disorder, or a disease relating to axial stem cells; or regenerative treatment of a cell, tissue, organ or body, comprising the step of administering a composition, preparation or kit of claim 14, wherein said method is an in vitro, ex vivo or in vivo method.

18. A method for screening a candidate compound for activity against a disease of the peripheral nervous system or a disease relating to axial stem cells, or for neurotoxicity screening said method comprising administering a composition, preparation or kit of claim 14 to a sample or subject.

19. The method according to claim 18, wherein said method is an in vitro, ex vivo or in vivo method.

20. (canceled)

21. The method for producing axial stem cells according to claim 1, wherein prior to step (a) said pluripotent cells are maintained in a suitable pluripotent cell media, wherein said suitable pluripotent cell media is replaced with RPMI 1640 medium supplemented with B27 supplement with or without vitamin A for said producing.

22. The method for producing axial stem cells according to claim 2, wherein said inhibitor of GSK3b protein is CHIR99021.

23. The method for producing axial stem cells according to claim 22, wherein said CHIR99021 is used at a concentration from about 5 μM to about 10 μM; wherein said CHIR99021 inhibitor is used for about 24 hours.

24. The method for producing axial stem cells according to claim 1, wherein said passaging is carried out for at least about 3 to 9 times, wherein said passaging is a serial passaging, wherein said passaging comprises re-seeding the cells derived from the step (b) at a lower density into a fresh serum-free medium.

25. The method according to claim 17, wherein said disease relating to axial stem cells, comprises a muscle-, motor neuron-, peripheral neuron-, sensory neuron cartilage-, or tendon-related disease.


SEQUENCE LISTING (39 sequences)

SEQ ID NO: 1 - DNA, 20 bp, Artificial Sequence
  GAPDH, forward primer
cgctctctgctcctcctgtt

SEQ ID NO: 2 - DNA, 20 bp, Artificial Sequence
  GAPDH, reverse primer
ccatggtgtctgagcgatgt

SEQ ID NO: 3 - DNA, 19 bp, Artificial Sequence
  MNX1, forward primer
gctcaacaagtacctgtcg

SEQ ID NO: 4 - DNA, 20 bp, Artificial Sequence
  MNX1, reverse primer
caaatcttcacctgggtctc

SEQ ID NO: 5 - DNA, 19 bp, Artificial Sequence
  PRPH, forward primer
tacaagtccaagtacgcgg

SEQ ID NO: 6 - DNA, 19 bp, Artificial Sequence
  PRPH, reverse primer
tcgttcatctcctgcttgg

SEQ ID NO: 7 - DNA, 19 bp, Artificial Sequence
  POU4F1, forward primer
ctccctgagcacaagtacc

SEQ ID NO: 8 - DNA, 19 bp, Artificial Sequence
  POU4F1, reverse primer
ttgaaaggatggctcttgc

SEQ ID NO: 9 - DNA, 21 bp, Artificial Sequence
  RUNX2, forward primer
accttgaccataaccgtcttc

SEQ ID NO: 10 - DNA, 19 bp, Artificial Sequence
  RUNX2, reverse primer
gcttctgtctgtgccttct

SEQ ID NO: 11 - DNA, 19 bp, Artificial Sequence
  BGLAP, forward primer
ctttgtgtccaagcaggag

SEQ ID NO: 12 - DNA, 19 bp, Artificial Sequence
  BGLAP, reverse primer
ctcccagccattgatacag

SEQ ID NO: 13 - DNA, 21 bp, Artificial Sequence
  COL1A1, forward primer
ctggaagagtggagagtactg

SEQ ID NO: 14 - DNA, 20 bp, Artificial Sequence
  COL1A1, reverse primer
gtctccatgttgcagaagac

SEQ ID NO: 15 - DNA, 19 bp, Artificial Sequence
  NKX3-2, forward primer
ccagagacaatacctgccc

SEQ ID NO: 16 - DNA, 19 bp, Artificial Sequence
  NKX3-2, reverse primer
aatagtaggagggctgcag

SEQ ID NO: 17 - DNA, 19 bp, Artificial Sequence
  COMP, forward primer
caagaccaggatggagacg

SEQ ID NO: 18 - DNA, 19 bp, Artificial Sequence
  COMP, reverse primer
caagaccaggatggagacg

SEQ ID NO: 19 - DNA, 21 bp, Artificial Sequence
  ACAN, forward primer
ctgaccagactgtcagatacc

SEQ ID NO: 20 - DNA, 21 bp, Artificial Sequence
  ACAN, reverse primer
ctgaccagactgtcagatacc

SEQ ID NO: 21 - protein, 317 aa, Homo sapiens
  MISC_FEATURE (1)..(317): SOX-2, UniProtKB Accession Number P48431
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 22 - protein, 360 aa, Homo sapiens
  MISC_FEATURE (1)..(360): POU5F1/OCT4, UniProtKB Accession Number Q01860-1
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 23 - protein, 305 aa, Homo sapiens
  MISC_FEATURE (1)..(305): NANOG, UniProtKB Accession Number Q9H9S0-1
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 24 - protein, 435 aa, Homo sapiens
  MISC_FEATURE (1)..(435): TBXT, UniProtKB Accession Number O15178-1
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 25 - protein, 313 aa, Homo sapiens
  MISC_FEATURE (1)..(313): CDX2, UniProtKB Accession Number Q99626-1
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVPGSVPGVLGPTGGVLNPTVTQ

SEQ ID NO: 26 - protein, 232 aa, Homo sapiens
  MISC_FEATURE (1)..(232): MIXL1, UniProtKB Accession Number Q9H2W2-1
MATAESRALQFAEGAAFPAYRAPHAGGALLPPPSPAAALLPAPPAGPGPATFAGFLGRDPGPAPPPPASLGSPAPPKGAAAPSASQRRKRTSFSAEQLQLLELVFRRTRYPDIHLRERLAALTLLPESRIQVWFQNRRAKSRRQSGKSFQPLARPEIILNHCAPGTETKCLKPQLPLEVDVNCLPEPNGVGGGISDSSSQGQNFETCSPLSEDIGSKLDSWEEHIFSAFGNF

SEQ ID NO: 27 - protein, 422 aa, Homo sapiens [sequence omitted]

SEQ ID NO: 28 - protein, 420 aa, Homo sapiens
  MISC_FEATURE (1)..(420): GSK3B, UniProtKB Accession Number P49841-1
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 29 - protein, 288 aa, Homo sapiens
  MISC_FEATURE (1)..(288): FGF2, UniProtKB Accession Number P09038-4
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 30 - protein, 390 aa, Homo sapiens
  MISC_FEATURE (1)..(390): TGFB1, UniProtKB Accession Number P01137-1
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 31 - protein, 250 aa, Homo sapiens
  MISC_FEATURE (1)..(250): LIN28B, UniProtKB Accession Number Q6ZN17-1
MAEGGASKGGGEEPGKLPEPAEEESQVLRGTGHCKWFNVRMGFGFISMINREGSPLDIPVDVFVHQSKLFMEGFRSLKEGEPVEFTFKKSSKGLESIRVTGPGGSPCLGSERRPKGKTLQKRKPKGDRCYNCGGLDHHAKECSLPPQPKKCHYCQSIMHMVANCPHKNVAQPPASSQGRQEAESQPCTSTLPREVGGGHGCTSPPFPQEARAEISERSGRSPQEASSTKSSIAPEEQSKKGPSVQKRKKT

SEQ ID NO: 32 - protein, 464 aa, Homo sapiens
  MISC_FEATURE (1)..(464): MYCN, UniProtKB Accession Number P04198-1
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 33 - protein, 532 aa, Homo sapiens
  MISC_FEATURE (1)..(532): ZIC2, UniProtKB Accession Number O95409-1
MLLDAGPQFPAIGVGSFARHHHHSAAAAAAAAAEMQDRELSLAAAQNGFVDSAAAHMGAFKLNPGAHELSPGQSSAFTSQGPGAYPGSAAAAAAAAALGPHAAHVGSYSGPPFNSTRDFLFRSRGFGDSAPGGGQHGLFGPGAGGLHHAHSDAQGHLLFPGLPEQHGPHGSQNVLNGQMRLGLPGEVFGRSEQYRQVASPRTDPYSAAQLHNQYGPMNMNMGMNMAAAAAHHHHHHHHHPGAFFRYMRQQCIKQELICKWIDPEQLSNPKKSCNKTFSTMHELVTHVSVEHVGGPEQSNHVCFWEECPREGKPFKAKYKLVNHIRVHTGEKPFPCPFPGCGKVFARSENLKIHKRTHTGEKPFQCEFEGCDRRFANSSDRKKHMHVHTSDKPYLCKMCDKSYTHPSSLRKHMKVHESSPQGSESSPAASSGYESSTPPGLVSPSAEPQSSSNLSPAAAAAAAAAAAAAAAVSAVHRGGGSGSGGAGGGSGGGSGSGGGGGGAGGGGGGSSGGGSGTAGGHSGLSSNFNEWYV

SEQ ID NO: 34 - protein, 501 aa, Homo sapiens
  MISC_FEATURE (1)..(501): IRX3, UniProtKB Accession Number P78415-1
MSFPQLGYQYIRPLYPSERPGAAGGSGGSAGARGGLGAGASELNASGSLSNVLSSVYGAPYAAAAAAAAAQGYGAFLPYAAELPIFPQLGAQYELKDSPGVQHPAAAAAFPHPHPAFYPYGQYQFGDPSRPKNATRESTSTLKAWLNEHRKNPYPTKGEKIMLAIITKMTLTQVSTWFANARRRLKKENKMTWAPRSRTDEEGNAYGSEREEEDEEEDEEDGKRELELEEEELGGEEEDTGGEGLADDDEDEEIDLENLDGAATEPELSLAGAARRDGDLGLGPISDSKNSDSEDSSEGLEDRPLPVLSLAPAPPPVAVASPSLPSPPVSLDPCAPAPAPASALQKPKIWSLAETATSPDNPRRSPPGAGGSPPGAAVAPSALQLSPAAAAAAAHRLVSAPLGKFPAWTNRPFPGPPPGPRLHPLSLLGSAPPHLLGLPGAAGHPAAAAAFARPAEPEGGTDRCSALEVEKKLLKTAFQPVPRRPQNHLDAALVLSALSSS

SEQ ID NO: 35 - protein, 391 aa, Homo sapiens
  MISC_FEATURE (1)..(391): SOX1, UniProtKB Accession Number O00570-1
MYSMMMETDLHSPGGAQAPTNLSGPAGAGGGGGGGGGGGGGGGAKANQDRVKRPMNAFMVWSRGQRRKMAQENPKMHNSEISKRLGAEWKVMSEAEKRPFIDEAKRLRALHMKEHPDYKYRPRRKTKTLLKKDKYSLAGGLLAAGAGGGGAAVAMGVGVGVGAAAVGQRLESPGGAAGGGYAHVNGWANGAYPGSVAAAAAAAAMMQEAQLAYGQHPGAGGAHPHAHPAHPHPHHPHAHPHNPQPMHRYDMGALQYSPISNSQGYMSASPSGYGGLPYGAAAAAAAAAGGAHQNSAVAAAAAAAAASSGALGALGSLVKSEPSGSPPAPAHSRAPCPGDLREMISMYLPAGEGGDPAAAAAAAAQSRLHSLPQHYQGAGAGVNGTVPLTHI

SEQ ID NO: 36 - protein, 441 aa, Homo sapiens
  MISC_FEATURE (1)..(441): SOX11, UniProtKB Accession Number P35716-1
MVQQAESLEAESNLPREALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKMDPSAKPSASQSPEKSAAGGGGGSAGGGAGGAKTSKGSSKKCGKLKAPAAAGAKAGAGKAAQSGDYGGAGDDYVLGSLRVSGSGGGGAGKTVKCVFLDEDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSSSSSGSSSGSSGEDADDLMFDLSLNFSQSAHSASEQQLGGGAAAGNLSLSLVDKDLDSFSEGSLGSHFEFPDYCTPELSEMIAGDWLEANFSDLVFTY

SEQ ID NO: 37 - protein, 371 aa, Homo sapiens
  MISC_FEATURE (1)..(371): NKX2-1, UniProtKB Accession Number P43699-1
MSMSPKHTTPFSVSDILSPLEESYKKVGMEGGGLGAPLAAYRQGQAAPPTAAMQQHAVGHHGAVTAAYHMTAAGVPQLSHSAVGGYCNGNLGNMSELPPYQDTMRNSASGPGWYGANPDPRFPAISRFMGPASGMNMSGMGGLGSLGDVSKNMAPLPSAPRRKRRVLFSQAQVYELERRFKQQKYLSAPEREHLASMIHLTPTQVKIWFQNHRYKMKRQAKDKAAQQQLQQDSGGGGGGGGTGCPQQQQAQQQSPRRVAVPVLVKDGKPCQAGAPAPGAASLQGHAQQQAQHQAQAAQAAAAAISVGSGGAGLGAHPGHQPGSAGQSPDLAHHAASPAALQGQVSSLSHLNSSGSDYGTMSCSTLLYGRTW

SEQ ID NO: 38 - protein, 868 aa, Homo sapiens
  MISC_FEATURE (1)..(868): TRIM71, UniProtKB Accession Number Q2Q1W2-1
MASFPETDFQICLLCKEMCGSPAPLSSNSSASSSSSQTSTSSGGGGGGPGAAARRLHVLPCLHAFCRPCLEAHRLPAAGGGAAGEPLKLRCPVCDQKVVLAEAAGMDALPSSAFLLSNLLDAVVATADEPPPKNGRAGAPAGAGGHSNHRHHAHHAHPRASASAPPLPQAPQPPAPSRSAPGGPAASPSALLLRRPHGCSSCDEGNAASSRCLDCQEHLCDNCVRAHQRVRLTKDHYIERGPPGPGAAAAAQQLGLGPPFPGPPFSILSVFPERLGFCQHHDDEVLHLYCDTCSVPICRECTMGRHGGHSFIYLQEALQDSRALTIQLLADAQQGRQAIQLSIEQAQTVAEQVEMKAKVVQSEVKAVTARHKKALEERECELLWKVEKIRQVKAKSLYLQVEKLRQNLNKLESTISAVQQVLEEGRALDILLARDRMLAQVQELKTVRSLLQPQEDDRVMFTPPDQALYLAIKSFGFVSSGAFAPLTKATGDGLKRALQGKVASFTVIGYDHDGEPRLSGGDLMSAVVLGPDGNLFGAEVSDQQNGTYVVSYRPQLEGEHLVSVTLCNQHIENSPFKVVVKSGRSYVGIGLPGLSFGSEGDSDGKLCRPWGVSVDKEGYIIVADRSNNRIQVFKPCGAFHHKFGTLGSRPGQFDRPAGVACDASRRIVVADKDNHRIQIFTFEGQFLLKFGEKGTKNGQFNYPWDVAVNSEGKILVSDTRNHRIQLFGPDGVFLNKYGFEGALWKHFDSPRGVAFNHEGHLVVTDFNNHRLLVIHPDCQSARFLGSEGTGNGQFLRPQGVAVDQEGRIIVADSRNHRVQMFESNGSFLCKFGAQGSGFGQMDRPSGIAITPDGMIVVVDFGNNRILVF

SEQ ID NO: 39 - protein, 325 aa, Homo sapiens
  MISC_FEATURE (1)..(325): FOXB1, UniProtKB Accession Number Q99853-1
MPRPGRNTYSDQKPPYSYISLTAMAIQSSPEKMLPLSEIYKFIMDRFPYYRENTQRWQNSLRHNLSFNDCFIKIPRRPDQPGKGSFWALHPSCGDMFENGSFLRRRKRFKVLKSDHLAPSKPADAAQYLQQQAKLRLSALAASGTHLPQMPAAAYNLGGVAQPSGFKHPFAIENIIAREYKMPGGLAFSAMQPVPAAYPLPNQLTTMGSSLGTGWPHVYGSAGMIDSATPISMASGDYSAYGVPLKPLCHAAGQTLPAIPVPIKPTPAAVPALPALPAPIPTLLSNSPPSLSPTSSQTATSQSSPATPSETLTSPASALHSVAVH